(19) United States
(12) Patent Application Publication
(10) Pub. No.: US 2022/0233264 A1
(43) Pub. Date: Jul. 28, 2022

(54) MANIPULATION OF AN ELONGATED MEDICAL DEVICE

(71) Applicant: Corindus, Inc., Waltham, MA (US)

(72) Inventors: Eric Klem, Lexington, MA (US); Cameron Canale, Groton, MA (US); Andrew Clark, Waltham, MA (US); Omid Saber, Waltham, MA (US); Saeed Sokhanvar, Belmont, MA (US); Steven J. Blacker, Framingham, MA (US); Per Bergman, West Roxbury, MA (US); Gary Kappel, Acton, MA (US); Peter Falb, Hingham, MA (US); Paul Gregory, Watertown, MA (US); Robert Payne, Wellesley, MA (US)

(21) Appl. No.: 17/597,364

(22) PCT Filed: Jul. 14, 2020
(86) PCT No.: PCT/US20/41923

§ 371 Date: Jan. 4, 2022

Related U.S. Application Data
(60) Provisional application No. 62/874,173, filed on Jul. 15, 2019.

(51) Int. Cl. A61B 34/35; A61B 90/00; A61B 34/37; A61B 34/00

(52) U.S. Cl. CPC A61B 34/35; A61B 90/37; A61B 2017/00477; A61B 34/74; A61B 34/37

(57) ABSTRACT

An EMD drive system includes an on-device adapter removably fixed to a shaft of an EMD. The on-device adapter received in a cassette. The cassette is removably secured to a drive module. The drive module is operatively coupled to the on-device adapter to move the on-device adapter and EMD together.

### CROSS-REFERENCE TO RELATED PATENT APPLICATIONS

This application claims the benefit of Provisional Application No. 62/874,173 (Atty Dkt C130-338) entitled MANIPULATION OF AN ELONGATED MEDICAL DEVICE and filed on Jul. 15, 2019.

### FIELD

The present invention relates generally to the field of robotic medical procedure systems and, in particular, to apparatus and methods for robotically controlling the movement and operation of elongated medical devices.

### BACKGROUND

Catheters and other elongated medical devices (EMDs) may be used for minimally invasive medical procedures for the diagnosis and treatment of diseases of various vascular systems, including neurovascular intervention (NVI) also known as neurointerventional surgery, percutaneous coronary intervention (PCI) and peripheral vascular intervention (PVI). These procedures typically involve navigating a guidewire through the vasculature, and via the guidewire advancing a catheter to deliver therapy. The catheterization procedure starts by gaining access into the appropriate vessel, such as an artery or vein, with an introducer sheath using standard percutaneous techniques. Through the introducer sheath, a sheath or guide catheter is then advanced over a diagnostic guidewire to a primary location such as an internal carotid artery for NVI, a coronary ostium for PCI, or a superficial femoral artery for PVI. A guidewire suitable for the vasculature is then navigated through the sheath or guide catheter to a target location in the vasculature. In certain situations, such as in tortuous anatomy, a support catheter or microcatheter is inserted over the guidewire to assist in navigating the guidewire. The physician or operator may use an imaging system (e.g., fluoroscope) to obtain a cine with a contrast injection and select a fixed frame for use as a roadmap to navigate the guidewire or catheter to the target location, for example, a lesion. Contrast-enhanced images are also obtained while the physician delivers the guidewire or catheter so that the physician can verify that the device is moving along the correct path to the target location. While observing the anatomy using fluoroscopy, the physician manipulates the proximal end of the guidewire or catheter to direct the distal tip into the appropriate vessels toward the lesion or target anatomical location and avoid advancing into side branches.

Robotic catheter-based procedure systems have been developed that may be used to aid a physician in performing catheterization procedures such as, for example, NVI, PCI and PVI. Examples of NVI procedures include coil embolization of aneurysms, liquid embolization of arteriovenous malformations and mechanical thrombectomy of large vessel occlusions in the setting of acute ischemic stroke. In an NVI procedure, the physician uses a robotic system to gain target lesion access by controlling the manipulation of a neurovascular guidewire and microcatheter to deliver the therapy to restore normal blood flow. Target access is enabled by the sheath or guide catheter but may also require an intermediate catheter for more distal territory or to provide adequate support for the microcatheter and guidewire. The distal tip of a guidewire is navigated into, or past, the lesion depending on the type of lesion and treatment. For treating aneurysms, the microcatheter is advanced into the lesion and the guidewire is removed and several embolization coils are deployed into the aneurysm through the microcatheter and used to block blood flow into the aneurysm. For treating arteriovenous malformations, a liquid embolic is injected into the malformation via a microcatheter. Mechanical thrombectomy to treat vessel occlusions can be achieved either through aspiration and/or use of a stent retriever. Depending on the location of the clot, aspiration is either done through an aspiration catheter, or through a microcatheter for smaller arteries. Once the aspiration catheter is at the lesion, negative pressure is applied to remove the clot through the catheter. Alternatively, the clot can be removed by deploying a stent retriever through the microcatheter. Once the clot has integrated into the stent retriever, the clot is retrieved by retracting the stent retriever and microcatheter (or intermediate catheter) into the guide catheter.

In PCI, the physician uses a robotic system to gain lesion access by manipulating a coronary guidewire to deliver the therapy and restore normal blood flow. The access is enabled by seating a guide catheter in a coronary ostium. The distal tip of the guidewire is navigated past the lesion and, for complex anatomies, a microcatheter may be used to provide adequate support for the guidewire. The blood flow is restored by delivering and deploying a stent or balloon at the lesion. The lesion may need preparation prior to stenting, by either delivering a balloon for pre-dilation of the lesion, or by performing atherectomy using, for example, a laser or rotational atherectomy catheter and a balloon over the guidewire. Diagnostic imaging and physiological measurements may be performed to determine appropriate therapy by using imaging catheters or fractional flow reserve (FFR) measurements.

In PVI, the physician uses a robotic system to deliver the therapy and restore blood flow with techniques similar to NVI. The distal tip of the guidewire is navigated past the lesion and a microcatheter may be used to provide adequate support for the guidewire for complex anatomies. The blood flow is restored by delivering and deploying a stent or balloon to the lesion. As with PCI, lesion preparation and diagnostic imaging may be used as well.

When support at the distal end of a catheter or guidewire is needed, for example, to navigate tortuous or calcified vasculature, to reach distal anatomical locations, or to cross hard lesions, an over-the-wire (OTW) catheter or coaxial system is used. An OTW catheter has a lumen for the guidewire that extends the full length of the catheter. This provides a relatively stable system because the guidewire is supported along the whole length. This system, however, has some disadvantages, including higher friction, and longer overall length compared to rapid-exchange catheters (see below). Typically to remove or exchange an OTW catheter while maintaining the position of the indwelling guidewire, the exposed length (outside of the patient) of guidewire must be longer than the OTW catheter. A 300 cm long guidewire is typically sufficient for this purpose and is often referred to as an exchange length guidewire. Due to the length of the guidewire, two operators are needed to remove or exchange an OTW catheter. This becomes even more challenging if a triple coaxial, known in the art as a tri-axial system, is used (quadruple coaxial catheters have also been known to be used). However, due to its stability, an OTW system is often used in NVI and PVI procedures. On the other hand, PCI procedures often use rapid exchange (or monorail) catheters. The guidewire lumen in a rapid exchange catheter runs only through a distal section of the catheter, called the monorail or rapid exchange (RX) section. With a RX system, the operator manipulates the interventional devices parallel to each other (as opposed to with an OTW system, in which the devices are manipulated in a serial configuration), and the exposed length of guidewire only needs to be slightly longer than the RX section of the catheter. A rapid exchange length guidewire is typically 180-200 cm long. Given the shorter length guidewire and monorail, RX catheters can be exchanged by a single operator. However, RX catheters are often inadequate when more distal support is needed.

### SUMMARY

An EMD drive system includes an on-device adapter removably fixed to a shaft of an EMD. The on-device adapter received in a cassette. The cassette is removably secured to a drive module. The drive module is operatively coupled to the on-device adapter to move the on-device adapter and EMD together.

In one embodiment an EMD drive system includes a collet removably fixed to an EMD. The EMD, fixed to the collet, is radially loaded into a robotic drive. An EMD support is removably applied to the EMD from a non-axial direction; and the robotic drive is operatively coupled to the collet to translate and/or rotate the collet and EMD

In one embodiment a robotic system includes a robotic drive including a base having a drive coupler. A cassette is removably secured to the base. A collet in the cassette is removably fixed to an EMD. The collet has a driven member being operatively coupled to the drive coupler; and the robotic drive includes a motor operatively coupled to the collet to move the collet

In one embodiment a robotic system includes a collet having a first portion having a first collet coupler connected thereto and a second portion having a second collet coupler connected thereto. An EMD is removably located within a pathway defined by the collet. A robotic drive including a base having a first motor and a second motor operatively continuously coupled to both the first collet coupler and the second collet coupler respectively to operatively pinch and unpinch the EMD in the pathway and to rotate the EMD.

In one embodiment a collet includes an inner member defining a pathway receiving an EMD and an outer member. A plurality of engagement members releasably engage the EMD as the inner member is moved relative to the outer member.

In one embodiment an EMD drive system includes a collet having a collet first member having a first engagement portion. The collet has a second member that is driven. A collet engagement member has a second engagement portion. The collet first member and the collet engagement member move between an engaged position and a disengaged position. The first engagement portion engages the second engagement portion as the collet first member and collet engagement member are moved to the engaged position. Rotation of the collet first member with respect to the collet second member in a first direction in the engaged position pinches an EMD within the collet and rotation of the collet first member with respect to the collect second member in a second direction opposite the first direction unpinches the EMD within the collet.

In another embodiment an EMD robotic drive system rotating and translating an EMD with reset instructions, includes a drive module controlled by a control system, the drive module including; a first actuator operatively rotating a first shaft and/or a second shaft; a second actuator operatively translating the first shaft along its longitudinal axis relative to the second shaft from a first position to a second position; a first tire assembly operatively attached to the first shaft; a second tire assembly operatively attached to a second shaft; a third actuator operatively moving the first tire assembly toward and away from the second tire assembly gripping and ungripping an EMD having a longitudinal axis from between the first tire assembly and the second tire assembly. The translation of the first shaft relative to the second shaft rotates the EMD about the longitudinal axis of the EMD, and rotation of the first shaft and/or second shaft translates the EMD along the longitudinal axis of the EMD. A control system provides reset instructions to the third actuator to ungrip the EMD; the second actuator to move the first tire assembly relative to the second tire assembly to a reset position; and the third actuator to grip the EMD.

In still another embodiment an EMD robotic drive system comprising a drive module including a first actuator operatively rotating a first shaft and/or a second shaft; a second actuator operatively translating the first shaft along its longitudinal axis relative to the second shaft from a first position to a second position; a first tire assembly removably attached to the first shaft; a second tire assembly removably attached to a second shaft. An EMD having a longitudinal axis is positioned at a first location between the first tire assembly and the second tire assembly. Rotation of the first shaft translates an EMD along its longitudinal axis between the first tire assembly and the second tire assembly; and rotation of the second shaft rotates the EMD about its longitudinal axis. A third actuator operatively moves the first tire assembly toward and away from the second tire assembly gripping and ungripping the EMD from between the first tire assembly and the second tire assembly. A holding clamp releasably clamps a portion of the EMD spaced from the first tire and the second tire along the longitudinal axis of the EMD.

In one embodiment an EMD robotic drive system includes a first actuator operatively rotating a first shaft and/or a second shaft. A second actuator operatively translates the first shaft along its longitudinal axis relative to the second shaft from a first position to a second position. A first tire assembly is operatively attached to the first shaft. A second tire assembly is operatively attached to a second shaft. A third actuator operatively moves the first tire assembly toward and away from the second tire assembly gripping and ungripping an EMD having a longitudinal axis from between the first tire assembly and the second tire assembly. Translation of the first shaft relative to the second shaft rotates the EMD about the longitudinal axis of the EMD, and rotation of the first shaft and/or second shaft translates the EMD along the longitudinal axis of the EMD. The first actuator moves with the first shaft as the first shaft is moved along its longitudinal axis away from a home position.

In one embodiment a method of robotically moving an EMD includes pinching a shaft of an EMD in an on-device adapter. Removably securing the on-device adapter into a cassette. Removably securing the cassette to a drive module; and robotically moving the on-device adapter and the EMD together in translation along a longitudinal axis of the EMD and/or rotation about the longitudinal axis of the EMD. In a further aspect the method includes unpinching the EMD in the on-device adapter with an actuator when the on-device adapter is secured in the cassette. In a further aspect the method includes unpinching the EMD is robotically controlled with an actuator.

### BRIEF DESCRIPTION OF THE DRAWINGS

FIG. 1 is a schematic view of an exemplary catheter procedure system in accordance with an embodiment.

FIG. 2 is a schematic block diagram of an exemplary catheter procedure system in accordance with an embodiment.

FIG. 3 is an isometric view of an exemplary bedside system of a catheter procedure system in accordance with an embodiment.

FIG. 4A is an exploded isometric view of a device module with a load sensing system and of a cassette that can receive an on-device adapter with an EMD in accordance with an embodiment.

FIG. 4B is an isometric view of a cassette with an on-device adapter with an EMD in accordance with an embodiment.

FIG. 4C is an exploded isometric view of a cassette showing first component and second component of an isolated component.

FIG. 4D is an exploded isometric view of the underside of a cassette and its connection to the drive module.

FIG. 4E is a partial side view of FIG. 51 showing an on-device adapter with an EMD supported in an isolated component as part of a cassette.

FIG. 4F is a cross-sectional view of the embodiment of 4A in a position with the EMD in the cassette.

FIG. 4G is an isometric view of a cassette and a device support.

FIG. 4H is a close-up isometric view of a device module of FIG. 3.

FIG. 5A is an exploded isometric view of a drive module with drive module base component and load-sensed component.

FIG. 5B is a close-up top view of FIG. 5A showing the load-sensed component connected to a load sensor within the drive module base component.

FIG. 5C is a top view of a drive module with a load sensing system including an actuator to rotate and/or pinch/unpinch an EMD located outside the load-sensed component and bearing support of load-sensed component in at least one off-axis (non-measured) direction.

FIG. 5D is a side view of a drive module with a load sensing system including an actuator to rotate and/or pinch/unpinch an EMD located outside the load-sensed component and bearing support of load-sensed component in at least one off-axis (non-measured) direction.

FIG. 5E is an isometric view of a drive module including a load-sensed component and a drive module base component.

FIG. 6A is an exploded side view an EMD on-device adapter in accordance with an embodiment.

FIG. 6B is a side view of the assembled EMD on-device adapter of FIG. 6A.

FIG. 6C is an exploded isometric view an EMD on-device adapter in accordance with an embodiment.

FIG. 6D is a side view of the assembled EMD on-device adapter of FIG. 6C.

FIG. 7A is an on-device adapter in accordance with an embodiment.

FIG. 7B is an exploded view of the on-device adapter of FIG. 7A.

FIG. 7C is an isometric view taken from a generally proximal orientation of the on-device adapter of FIG. 7A.

FIG. 7D is an isometric view taken from a generally bottom orientation of the on-device adapter of FIG. 7A.

FIG. 7E is a cross section of the on-device adapter of FIG. 7A with the lever in the open position.

FIG. 7F is a cross section of the on-device adapter of FIG. 7A with the lever in the closed position.

FIG. 8A is an isometric view of an-device adapter with a catheter.

FIG. 8B is a schematic isometric view of a catheter embodiment used with the on-device adapter of FIG. 8A.

FIG. 9A is an isometric view of a collet.

FIG. 9B is an isometric view of an inner member of the collet of FIG. 9A.

FIG. 9C is a view of the collet of FIG. 9A taken generally along lines 9C-9C.

FIG. 9D is a top plan view of an inner member of the collet of FIG. 9A taken generally along lines 9D-9D in FIG. 9B.

FIG. 9E is a close-up view of the free end of the inner member of FIG. 9D.

FIG. 9F is a top plan view of an inner member of the collet of FIG. 9A taken generally along lines 9F-9F in FIG. 9B.

FIG. 9G is an isometric view of another collet.

FIG. 9H is a view of the collet of FIG. 9G taken generally along lines 9H-9H.

FIG. 9I is an isometric view of the inner member of FIG. 9G.

FIG. 10A is an isometric view of a cam-actuated collet.

FIG. 10B is an isometric exploded (assembly) view of FIG. 10A.

FIG. 10C.1 is a longitudinal cross-sectional view of FIG. 10A in the unpinched configuration.

FIG. 10C.2 is a transverse cross-sectional view of FIG. 10A in the unpinched configuration.

FIG. 10D.1 is a longitudinal cross-sectional view of FIG. 10A in the pinched configuration.

FIG. 10D.2 is a transverse cross-sectional view of FIG. 10A in the pinched configuration.

FIG. 11A is a longitudinal cross-sectional view of a flexure-actuated collet.

FIG. 11B is an assembled cross-sectional view of the flexure-actuated collet of FIG. 11A.

FIG. 11C is an exploded (assembly) view of the flexure-actuated collet of FIG. 11A.

FIG. 11D is an isometric cross-sectional view of the flexure-actuated collet of FIG. 11A.

FIG. 11E is an isometric view of a collar of the flexure-actuated collet of FIG. 11A.

FIG. 12A is an isometric view of a system that includes a double-gear collet-drive assembly.

FIG. 12B is a side view of the double-gear collet-drive assembly of FIG. 12A.

FIG. 12C is an isometric view of the double-gear collet-drive assembly of FIG. 12A.

FIG. 12D is an isometric exploded (clamshell) view showing two perspectives of the double-gear collet-drive assembly of FIG. 12A.

FIG. 12E is an isometric view showing select components of the double-gear collet-drive assembly of FIG. 12A.

FIG. 12F.1 is a longitudinal cross-sectional top view showing internal components of the double-gear collet-drive assembly of FIG. 12A in the unpinched configuration.

FIG. 12F.2 is a longitudinal cross-sectional top view showing internal components of the double-gear collet-drive assembly of FIG. 12A in the pinched configuration.

FIG. 13A is an isometric view of a double-gear sliding collet-drive system.

FIG. 13B.1 is a side view of the double-gear sliding collet-drive system of FIG. 13A in the proximal configuration.

FIG. 13B.2 is a side view of the double-gear sliding collet-drive system of FIG. 13A in the distal configuration.

FIG. 13C is a zoomed-in side view of the collet-and-rotational-drive assembly of FIG. 13A.

FIG. 13D.1 is a longitudinal cross-sectional side view showing internal components of the double-gear sliding collet-drive assembly of FIG. 13A in the unpinched configuration.

FIG. 13D.2 is a longitudinal cross-sectional side view showing internal components of the double-gear sliding collet-drive assembly of FIG. 13A in the pinched configuration.

FIG. 14A is an isometric view of a double-gear sliding collet-drive system with a reset mechanism.

FIG. 14B is a bottom view of the double-gear sliding collet-drive system with a reset mechanism of FIG. 14A.

FIG. 14C.1 is a top view with some critical components visible of the double-gear sliding collet-drive system with a reset mechanism of FIG. 14A with the collet locking.

FIG. 14C.2 is a top view with some critical components visible of the double-gear sliding collet-drive system with a reset mechanism of FIG. 14A with the EMD advancing.

FIG. 14C.3 is a top view with some critical components visible of the double-gear sliding collet-drive system with a reset mechanism of FIG. 14A with the collet unlocking.

FIG. 14C.4 is a top view with some critical components visible of the double-gear sliding collet-drive system with a reset mechanism of FIG. 14A with the EMD retracting.

FIG. 15A is an isometric view of a system that includes a bellows drive.

FIG. 15B is a zoomed-in isometric view of the drive blocks of FIG. 15A in an open configuration.

FIG. 15C is a zoomed-in isometric view of the drive blocks of FIG. 15A in a closed configuration.

FIG. 15D is a cross-sectional view of the device retainer of FIG. 15A in an open configuration.

FIG. 15E is a cross-sectional view of the device retainer of FIG. 15A in a closed configuration.

FIG. 15F is a zoomed-in isometric view of the holding blocks of FIG. 15A in an open configuration.

FIG. 15G is a zoomed-in isometric view of the holding blocks of FIG. 15A in a drive configuration.

FIG. 15H is a zoomed-in isometric view of the holding blocks of FIG. 15A in a pinched configuration.

FIG. 16A is an isometric exploded view of a compression-collet system.

FIG. 16B is an isometric assembled view of the compression-collet system of FIG. 16A.

FIG. 16C is a cross-sectional view showing the compression-collet system of FIG. 16A in an unloaded configuration.

FIG. 16D is a cross-sectional view showing the compression-collet system of FIG. 16A in a loaded configuration.

FIG. 17A is an isometric view (with phantom lines) of a plunger collet system.

FIG. 17B is a longitudinal cross-sectional view of the plunger collet system of FIG. 17A taken generally along lines 17A.1-17A.1 in FIG. 17A in the unpinched configuration.

FIG. 17C is a longitudinal cross-sectional view of the plunger collet system of FIG. 17A taken generally along lines 17A.1-17A.1 in FIG. 17A in the pinched configuration.

FIG. 18A is an isometric exploded assembly view of a plunger collet system with a circular disk housing.

FIG. 18B is an isometric view of a multi-plunger collet system.

FIG. 18C is an isometric view of a multi-plunger collet system with a single plunger collet assembly removed.

FIG. 18D is a side view of a multi-plunger collet system with phantom lines taken generally along lines 18D-18D in FIG. 18B.

FIG. 18E is longitudinal cross-sectional view of a multi-plunger collet in an unpinched configuration taken generally along lines 18E-18E in FIG. 18D.

FIG. 18F is longitudinal cross-sectional view of a multi-plunger collet in a pinched configuration taken generally along lines 18E-18E in FIG. 18D.

FIG. 18G is an isometric view of a multi-plunger collet system with six plungers oriented in the same direction and side and front views of an EMD in the pinched configuration.

FIG. 18H is an isometric view of a multi-plunger collet system with six plungers alternately oriented 180 degrees apart and side and front views of an EMD in the pinched configuration.

FIG. 18I is an isometric view of a multi-plunger collet system with six plungers progressively rotated 60 degrees apart and side and front views of an EMD in the pinched configuration.

FIG. 19A is an isometric view of an opposing pad collet having an inner housing and an outer housing.

FIG. 19B is a side cross-sectional view of an opposing pad collet in an unpinched configuration taken generally along lines 19B-19B in FIG. 19A.

FIG. 19C is a side cross-sectional view of an opposing pad collet in a pinched configuration taken generally along lines 19B-19B in FIG. 19A.

FIG. 19D is cross section and end view of the collet of FIG. 19A in a first position.

FIG. 19E is cross section and end view of the collet of FIG. 19A in a second position.

FIG. 19F is cross section and end view of the collet of FIG. 19A in a third position.

FIG. 19G is cross section and end view of the collet of FIG. 19A in a fourth position.

FIG. 20A is an isometric view of a collet-drive system with two drive modules.

FIG. 20B is a side view of a first drive module of a collet-drive system with two drive modules of FIG. 20A showing some internal components.

FIG. 20C is a plan view of a collet-drive system with two drive modules of FIG. 20A in a driving state.

FIG. 20D is a plan view of a collet-drive system with two drive modules of FIG. 20A in a collet lock state.

FIG. 20E is a plan view of a collet-drive system with two drive modules of FIG. 20A in a device exchange state.

FIG. 20F is a plan view of a collet-drive system with two drive modules of FIG. 20A in a state with collet pinched and tires gripped.

FIG. 20G is a plan view of a collet-drive system with two drive modules of FIG. 20A in a tire driving state.

FIG. 21A is a plan view of a collet-drive system with EMD support.

FIG. 21B is a plan view of a collet-drive system with EMD support of FIG. 21A with a clamp.

FIG. 21C is a plan view of a collet-drive system with EMD support of FIG. 21A with a proximal tires.

FIG. 21D is a plan view of a collet-drive system with EMD support of FIG. 21A with a distal tires.

FIG. 22A is right isometric view of a drive mechanism for actuating a pair of tires.

FIG. 22B is an exploded view of the drive mechanism of FIG. 22A

FIG. 22C is a left plan view of the drive mechanism of FIG. 22A with the tires in a neutral position.

FIG. 22D a left plan view of the drive mechanism of FIG. 22A with the tires in a second position.

FIG. 22E is a left plan view of the drive mechanism of FIG. 22A with a housing for the tires.

FIG. 22F is a left isometric view of the drive mechanism of FIG. 22A with the offset mechanism in a first configuration.

FIG. 22G is a top plan view of the mechanism from FIG. 22F with the engagement cam in an unclamped position and the tires in an engaged position.

FIG. 22H is a top plan view of the mechanism from FIG. 22F with the engagement cam in a clamped position and the tires in an engaged position.

FIG. 22I is a top plan view of the mechanism from FIG. 22F with the engagement cam in a clamped position and the tires in a disengaged position.

FIG. 22J is a top plan view of the mechanism from FIG. 22F with the engagement cam in an unclamped position and the tires in a disengaged position.

FIG. 22K is a schematic view of the eccentric assembly with the first tire assembly and second tire assembly gripping the EMD.

FIG. 22L is a schematic view of the eccentric assembly with the first tire assembly and second tire assembly not gripping the EMD.

FIG. 22M is an isometric view of the tire assemblies being installed onto couplers.

FIG. 22N is a cross sectional view of the tire assemblies and couplers.

FIG. 22O is a partial cross-sectional view of the tire assemblies and eccentric assembly.

FIG. 22P is a schematic cross-sectional view of the tire assemblies having a conical shape.

FIG. 22Q is a schematic cross-sectional view of the tire assemblies having a conical shape in an engaged position.

FIG. 22R is a front view of the tire assemblies being secured to the couplers with an installation member.

FIG. 22S is a front view of the tire assemblies with one tire assembly being removed from the coupler.

FIG. 22T is a close-up of one tire assembly being removed from the coupler.

FIG. 22U is a close-up isometric view of the tire assemblies.

FIG. 22V is a schematic cross sectional view of the tire assemblies and EMD in a first position.

FIG. 22W is a schematic cross sectional view of the tire assemblies and EMD in a second position.

FIG. 22X is a schematic cross sectional view of the tire assemblies and EMD in a third position.

### DETAILED DESCRIPTION OF THE EXAMPLE EMBODIMENTS

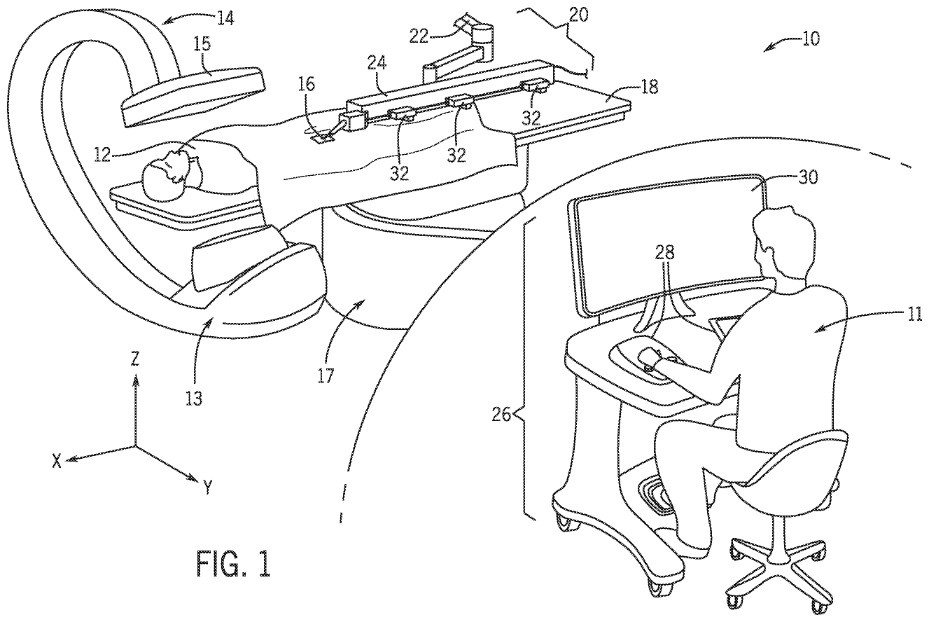

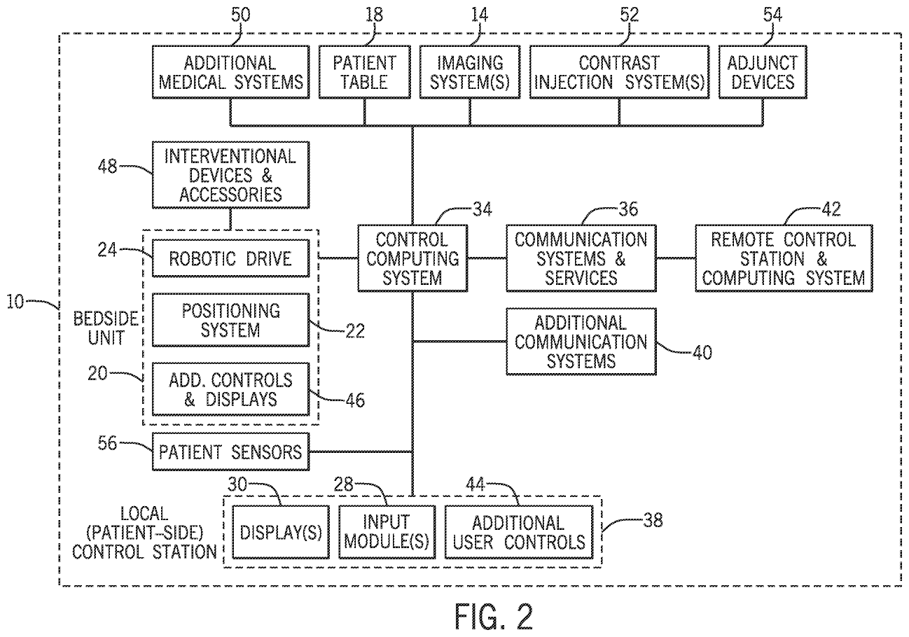

FIG. 1 is a perspective view of an exemplary catheter-based procedure system 10 in accordance with an embodiment. Catheter-based procedure system 10 may be used to perform catheter-based medical procedures, e.g., percutaneous intervention procedures such as a percutaneous coronary intervention (PCI) (e.g., to treat STEMI), a neurovascular interventional procedure (NVI) (e.g., to treat an emergent large vessel occlusion (ELVO)), peripheral vascular intervention procedures (PVI) (e.g., for critical limb ischemia (CLI), etc.). Catheter-based medical procedures may include diagnostic catheterization procedures during which one or more catheters or other elongated medical devices (EMDs) are used to aid in the diagnosis of a patient's disease. For example, during one embodiment of a catheter-based diagnostic procedure, a contrast media is injected onto one or more arteries through a catheter and an image of the patient's vasculature is taken. Catheter-based medical procedures may also include catheter-based therapeutic procedures (e.g., angioplasty, stent placement, treatment of peripheral vascular disease, clot removal, arterial venous malformation therapy, treatment of aneurysm, etc.) during which a catheter (or other EMD) is used to treat a disease. Therapeutic procedures may be enhanced by the inclusion of adjunct devices 54 (shown in FIG. 2) such as, for example, intravascular ultrasound (IVUS), optical coherence tomography (OCT), fractional flow reserve (FFR), etc. It should be noted, however, that one skilled in the art would recognize that certain specific percutaneous intervention devices or components (e.g., type of guidewire, type of catheter, etc.) may be selected based on the type of procedure that is to be performed. Catheter-based procedure system 10 can perform any number of catheter-based medical procedures with minor adjustments to accommodate the specific percutaneous intervention devices to be used in the procedure.

Catheter-based procedure system 10 includes, among other elements, a bedside unit 20 and a control station 26. Bedside unit 20 includes a robotic drive 24 and a positioning system 22 that are located adjacent to a patient 12. Patient 12 is supported on a patient table 18. The positioning system 22 is used to position and support the robotic drive 24. The positioning system 22 may be, for example, a robotic arm, an articulated arm, a holder, etc. The positioning system 22 may be attached at one end to, for example, a rail on the patient table 18, a base, or a cart. The other end of the positioning system 22 is attached to the robotic drive 24. The positioning system 22 may be moved out of the way (along with the robotic drive 24) to allow for the patient 12 to be placed on the patient table 18. Once the patient 12 is positioned on the patient table 18, the positioning system 22 may be used to situate or position the robotic drive 24 relative to the patient 12 for the procedure. In an embodiment, patient table 18 is operably supported by a pedestal 17, which is secured to the floor and/or earth. Patient table 18 is able to move with multiple degrees of freedom, for example, roll, pitch, and yaw, relative to the pedestal 17. Bedside unit 20 may also include controls and displays 46 (shown in FIG. 2). For example, controls and displays may be located on a housing of the robotic drive 24.

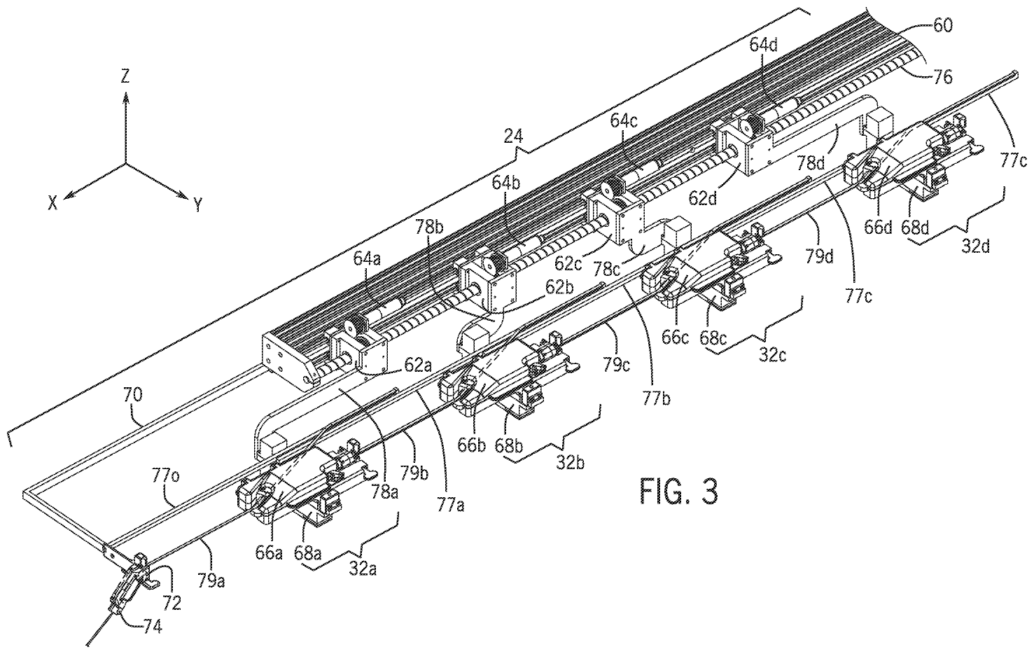

Generally, the robotic drive 24 may be equipped with the appropriate percutaneous interventional devices and accessories 48 (shown in FIG. 2) (e.g., guidewires, various types of catheters including balloon catheters, stent delivery systems, stent retrievers, embolization coils, liquid embolics, aspiration pumps, device to deliver contrast media, medicine, hemostasis valve adapters, syringes, stopcocks, inflation device, etc.) to allow the user or operator 11 to perform a catheter-based medical procedure via a robotic system by operating various controls such as the controls and inputs located at the control station 26. Bedside unit 20, and in particular robotic drive 24, may include any number and/or combination of components to provide bedside unit 20 with the functionality described herein. A user or operator 11 at control station 26 is referred to as the control station user or control station operator and referred to herein as user or operator. A user or operator at bedside unit 20 is referred to as bedside unit user or bedside unit operator. The robotic drive 24 includes a plurality of device modules 32a-d mounted to a rail or linear member 60 (shown in FIG. 3). The rail or linear member 60 guides and supports the device modules. Each of the device modules 32a-d may be used to drive an EMD such as a catheter or guidewire. For example, the robotic drive 24 may be used to automatically feed a guidewire into a diagnostic catheter and into a guide catheter in an artery of the patient 12. One or more devices, such as an EMD, enter the body (e.g., a vessel) of the patient 12 at an insertion point 16 via, for example, an introducer sheath.

Bedside unit 20 is in communication with control station 26, allowing signals generated by the user inputs of control station 26 to be transmitted wirelessly or via hardwire to bedside unit 20 to control various functions of bedside unit 20. As discussed below, control station 26 may include a control computing system 34 (shown in FIG. 2) or be coupled to the bedside unit 20 through a control computing system 34. Bedside unit 20 may also provide feedback signals (e.g., loads, speeds, operating conditions, warning signals, error codes, etc.) to control station 26, control computing system 34 (shown in FIG. 2), or both. Communication between the control computing system 34 and various components of the catheter-based procedure system 10 may be provided via a communication link that may be a wireless connection, cable connections, or any other means capable of allowing communication to occur between components. Control station 26 or other similar control system may be located either at a local site (e.g., local control station 38 shown in FIG. 2) or at a remote site (e.g., remote control station and computer system 42 shown in FIG. 2). Catheter procedure system 10 may be operated by a control station at the local site, a control station at a remote site, or both the local control station and the remote control station at the same time. At a local site, user or operator 11 and control station 26 are located in the same room or an adjacent room to the patient 12 and bedside unit 20. As used herein, a local site is the location of the bedside unit 20 and a patient 12 or subject (e.g., animal or cadaver) and the remote site is the location of a user or operator 11 and a control station 26 used to control the bedside unit 20 remotely. A control station 26 (and a control computing system) at a remote site and the bedside unit 20 and/or a control computing system at a local site may be in communication using communication systems and services 36 (shown in FIG. 2), for example, through the Internet. In an embodiment, the remote site and the local (patient) site are away from one another, for example, in different rooms in the same building, different buildings in the same city, different cities, or other different locations where the remote site does not have physical access to the bedside unit 20 and/or patient 12 at the local site.

Control station 26 generally includes one or more input modules 28 configured to receive user inputs to operate various components or systems of catheter-based procedure system 10. In the embodiment shown, control station 26 allows the user or operator 11 to control bedside unit 20 to perform a catheter-based medical procedure. For example, input modules 28 may be configured to cause bedside unit 20 to perform various tasks using percutaneous intervention devices (e.g., EMDs) interfaced with the robotic drive 24 (e.g., to advance, retract, or rotate a guidewire, advance, retract or rotate a catheter, inflate or deflate a balloon located on a catheter, position and/or deploy a stent, position and/or deploy a stent retriever, position and/or deploy a coil, inject contrast media into a catheter, inject liquid embolics into a catheter, inject medicine or saline into a catheter, aspirate on a catheter, or to perform any other function that may be performed as part of a catheter-based medical procedure). Robotic drive 24 includes various drive mechanisms to cause movement (e.g., axial and rotational movement) of the components of the bedside unit 20 including the percutaneous intervention devices.

In one embodiment, input modules 28 may include one or more touch screens, joysticks, scroll wheels, and/or buttons. In addition to input modules 28, the control station 26 may use additional user controls 44 (shown in FIG. 2) such as foot switches and microphones for voice commands, etc. Input modules 28 may be configured to advance, retract, or rotate various components and percutaneous intervention devices such as, for example, a guidewire, and one or more catheters or microcatheters. Buttons may include, for example, an emergency stop button, a multiplier button, device selection buttons and automated move buttons. When an emergency stop button is pushed, the power (e.g., electrical power) is shut off or removed to bedside unit 20. When in a speed control mode, a multiplier button acts to increase or decrease the speed at which the associated component is moved in response to a manipulation of input modules 28. When in a position control mode, a multiplier button changes the mapping between input distance and the output commanded distance. Device selection buttons allow the user or operator 11 to select which of the percutaneous intervention devices loaded into the robotic drive 24 are controlled by input modules 28. Automated move buttons are used to enable algorithmic movements that the catheter-based procedure system 10 may perform on a percutaneous intervention device without direct command from the user or operator 11. In one embodiment, input modules 28 may include one or more controls or icons (not shown) displayed on a touch screen (that may or may not be part of a display 30), that, when activated, causes operation of a component of the catheter-based procedure system 10. Input modules 28 may also include a balloon or stent control that is configured to inflate or deflate a balloon and/or deploy a stent. Each of the input modules 28 may include one or more buttons, scroll wheels, joysticks, touch screen, etc. that may be used to control the particular component or components to which the control is dedicated. In addition, one or more touch screens may display one or more icons (not shown) related to various portions of input modules 28 or to various components of catheter-based procedure system 10.

Control station 26 may include a display 30. In other embodiments, the control station 26 may include two or more displays 30. Display 30 may be configured to display information or patient specific data to the user or operator 11 located at control station 26. For example, display 30 may be configured to display image data (e.g., X-ray images, MRI images, CT images, ultrasound images, etc.), hemodynamic data (e.g., blood pressure, heart rate, etc.), patient record information (e.g., medical history, age, weight, etc.), lesion or treatment assessment data (e.g., IVUS, OCT, FFR, etc.). In addition, display 30 may be configured to display procedure specific information (e.g., procedural checklist, recommendations, duration of procedure, catheter or guidewire position, volume of medicine or contrast agent delivered, etc.). Further, display 30 may be configured to display information to provide the functionalities associated with control computing system 34 (shown in FIG. 2). Display 30 may include touch screen capabilities to provide some of the user input capabilities of the system.

Catheter-based procedure system 10 also includes an imaging system 14. Imaging system 14 may be any medical imaging system that may be used in conjunction with a catheter based medical procedure (e.g., non-digital X-ray, digital X-ray, CT, MRI, ultrasound, etc.). In an exemplary embodiment, imaging system 14 is a digital X-ray imaging device that is in communication with control station 26. In one embodiment, imaging system 14 may include a C-arm (shown in FIG. 1) that allows imaging system 14 to partially or completely rotate around patient 12 in order to obtain images at different angular positions relative to patient 12 (e.g., sagittal views, caudal views, anterior-posterior views, etc.). In one embodiment imaging system 14 is a fluoroscopy system including a C-arm having an X-ray source 13 and a detector 15, also known as an image intensifier.

Imaging system 14 may be configured to take X-ray images of the appropriate area of patient 12 during a procedure. For example, imaging system 14 may be configured to take one or more X-ray images of the head to diagnose a neurovascular condition. Imaging system 14 may also be configured to take one or more X-ray images (e.g., real time images) during a catheter-based medical procedure to assist the user or operator 11 of control station 26 to properly position a guidewire, guide catheter, microcatheter, stent retriever, coil, stent, balloon, etc. during the procedure. The image or images may be displayed on display 30. For example, images may be displayed on display 30 to allow the user or operator 11 to accurately move a guide catheter or guidewire into the proper position.

In order to clarify directions, a rectangular coordinate system is introduced with X, Y, and Z axes. The positive X axis is oriented in a longitudinal (axial) distal direction, that is, in the direction from the proximal end to the distal end, stated another way from the proximal to distal direction. The Y and Z axes are in a transverse plane to the X axis, with the positive Z axis oriented up, that is, in the direction opposite of gravity, and the Y axis is automatically determined by right-hand rule.

FIG. 2 is a block diagram of catheter-based procedure system 10 in accordance with an exemplary embodiment. Catheter-procedure system 10 may include a control computing system 34. Control computing system 34 may physically be, for example, part of control station 26 (shown in FIG. 1). Control computing system 34 may generally be an electronic control unit suitable to provide catheter-based procedure system 10 with the various functionalities described herein. For example, control computing system 34 may be an embedded system, a dedicated circuit, a general-purpose system programmed with the functionality described herein, etc. Control computing system 34 is in communication with bedside unit 20, communications systems and services 36 (e.g., Internet, firewalls, cloud services, session managers, a hospital network, etc.), a local control station 38, additional communications systems 40 (e.g., a telepresence system), a remote control station and computing system 42, and patient sensors 56 (e.g., electrocardiogram (ECG) devices, electroencephalogram (EEG) devices, blood pressure monitors, temperature monitors, heart rate monitors, respiratory monitors, etc.). The control computing system is also in communication with imaging system 14, patient table 18, additional medical systems 50, contrast injection systems 52 and adjunct devices 54 (e.g., IVUS, OCT, FFR, etc.). The bedside unit 20 includes a robotic drive 24, a positioning system 22 and may include additional controls and displays 46. As mentioned above, the additional controls and displays may be located on a housing of the robotic drive 24. Interventional devices and accessories 48 (e.g., guidewires, catheters, etc.) interface to the bedside system 20. In an embodiment, interventional devices and accessories 48 may include specialized devices (e.g., IVUS catheter, OCT catheter, FFR wire, diagnostic catheter for contrast, etc.) which interface to their respective adjunct devices 54, namely, an IVUS system, an OCT system, and FFR system, etc.

In various embodiments, control computing system 34 is configured to generate control signals based on the user's interaction with input modules 28 (e.g., of a control station 26 (shown in FIG. 1) such as a local control station 38 or a remote control station 42) and/or based on information accessible to control computing system 34 such that a medical procedure may be performed using catheter-based procedure system 10. The local control station 38 includes one or more displays 30, one or more input modules 28, and additional user controls 44. The remote control station and computing system 42 may include similar components to the local control station 38. The remote 42 and local 38 control stations can be different and tailored based on their required functionalities. The additional user controls 44 may include, for example, one or more foot input controls. The foot input control may be configured to allow the user to select functions of the imaging system 14 such as turning on and off the X-ray and scrolling through different stored images. In another embodiment, a foot input device may be configured to allow the user to select which devices are mapped to scroll wheels included in input modules 28. Additional communication systems 40 (e.g., audio conference, video conference, telepresence, etc.) may be employed to help the operator interact with the patient, medical staff (e.g., angio-suite staff), and/or equipment in the vicinity of the bedside.

Catheter-based procedure system 10 may be connected or configured to include any other systems and/or devices not explicitly shown. For example, catheter-based procedure system 10 may include image processing engines, data storage and archive systems, automatic balloon and/or stent inflation systems, medicine injection systems, medicine tracking and/or logging systems, user logs, encryption systems, systems to restrict access or use of catheter-based procedure system 10, etc.

As mentioned, control computing system 34 is in communication with bedside unit 20 which includes a robotic drive 24, a positioning system 22 and may include additional controls and displays 46, and may provide control signals to the bedside unit 20 to control the operation of the motors and drive mechanisms used to drive the percutaneous intervention devices (e.g., guidewire, catheter, etc.). The various drive mechanisms may be provided as part of a robotic drive 24. FIG. 3 is a perspective view of a robotic drive for a catheter-based procedure system 10 in accordance with an embodiment. In FIG. 3, a robotic drive 24 includes multiple device modules 32a-d coupled to a linear member 60. Each device module 32a-d is coupled to the linear member 60 via a stage 62a-d moveably mounted to the linear member 60. A device module 32a-d may be connected to a stage 62a-d using a connector such as an offset bracket 78a-d. In another embodiment, the device module 32a-d is directly mounted to the stage 62a-d. Each stage 62a-d may be independently actuated to move linearly along the linear member 60. Accordingly, each stage 62a-d (and the corresponding device module 32a-d coupled to the stage 62a-d) may independently move relative to each other and the linear member 60. A drive mechanism is used to actuate each stage 62a-d. In the embodiment shown in FIG. 3, the drive mechanism includes independent stage translation motors 64a-d coupled to each stage 62a-d and a stage drive mechanism 76, for example, a lead screw via a rotating nut, a rack via a pinion, a belt via a pinion or pulley, a chain via a sprocket, or the stage translation motors 64a-d may be linear motors themselves. In some embodiments, the stage drive mechanism 76 may be a combination of these mechanisms, for example, each stage 62a-d could employ a different type of stage drive mechanism. In an embodiment where the stage drive mechanism is a lead screw and rotating nut, the lead screw may be rotated and each stage 62a-d may engage and disengage from the lead screw to move, e.g., to advance or retract. In the embodiment shown in FIG. 3, the stages 62a-d and device modules 32a-d are in a serial drive configuration.

Each device module 32a-d includes a drive module 68a-d and a cassette 66a-d mounted on and coupled to the drive module 68a-d. In the embodiment shown in FIG. 3, each cassette 66a-d is mounted to the drive module 68a-d in a vertical orientation. In other embodiments, each cassette 66a-d may be mounted to the drive module 68a-d in other mounting orientations. Each cassette 66a-d is configured to interface with and support a proximal portion of an EMD (not shown). In addition, each cassette 66a-d may include elements to provide one or more degrees of freedom in addition to the linear motion provided by the actuation of the corresponding stage 62a-d to move linearly along the linear member 60. For example, the cassette 66a-d may include elements that may be used to rotate the EMD when the cassette is coupled to the drive module 68a-d. Each drive module 68a-d includes at least one coupler to provide a drive interface to the mechanisms in each cassette 66a-d to provide the additional degree of freedom. Each cassette 66a-d also includes a channel in which a device support 79a-d is positioned, and each device support 79a-d is used to prevent an EMD from buckling. A support arm 77a, 77b, and 77c is attached to each device module 32a, 32b, and 32c, respectively, to provide a fixed point for support of a proximal end of the device supports 79b, 79c, and 79d, respectively. The robotic drive 24 may also include a device support connection 72 connected to a device support 79, a distal support arm 70 and a support arm 77o. Support arm 77o is used to provide a fixed point for support of the proximal end of the distal most device support 79a housed in the distal most device module 32a. In addition, an introducer interface support (redirector) 74 may be connected to the device support connection 72 and an EMD (e.g., an introducer sheath). The configuration of robotic drive 24 has the benefit of reducing volume and weight of the drive robotic drive 24 by using actuators on a single linear member.

To prevent contaminating the patient with pathogens, healthcare staff use aseptic technique in a room housing the bedside unit 20 and the patient 12 or subject (shown in FIG. 1). A room housing the bedside unit 20 and patient 12 may be, for example, a cath lab or an angio suite. Aseptic technique consists of using sterile barriers, sterile equipment, proper patient preparation, environmental controls and contact guidelines. Accordingly, all EMDs and interventional accessories are sterilized and can only be in contact with either sterile barriers or sterile equipment. In an embodiment, a sterile drape (not shown) is placed over the non-sterile robotic drive 24. Each cassette 66a-d is sterilized and acts as a sterile interface between the draped robotic drive 24 and at least one EMD. Each cassette 66a-d can be designed to be sterile for single use or to be re-sterilized in whole or part so that the cassette 66a-d or its components can be used in multiple procedures.

Distal and Proximal: The terms distal and proximal define relative locations of two different features. With respect to a robotic drive the terms distal and proximal are defined by the position of the robotic drive in its intended use relative to a patient. When used to define a relative position, the distal feature is the feature of the robotic drive that is closer to the patient than a proximal feature when the robotic drive is in its intended in-use position. Within a patient, any vasculature landmark further away along the path from the access point is considered more distal than a landmark closer to the access point, where the access point is the point at which the EMD enters the patient. Similarly, the proximal feature is the feature that is farther from the patient than the distal feature when the robotic drive in its intended in-use position. When used to define direction, the distal direction refers to a path on which something is moving or is aimed to move or along which something is pointing or facing from a proximal feature toward a distal feature and/or patient when the robotic drive is in its intended in-use position. The proximal direction is the opposite direction of the distal direction. By way of examples referring to FIG. 1, a robotic device is shown from the viewpoint of an operator facing a patient. In this arrangement, the distal direction is along the positive X coordinate axis and the proximal direction is along the negative X coordinate axis. Referring to FIG. 3, the EMD is moved in a distal direction on a path toward a patient through the introducer interface support 74 which defines the distal end of the robotic drive 24. The proximal end of the robotic drive 24 is the point furthest from the distal end along the negative X axis. Referring to FIG. 3, the most distal drive module is the drive module 32a closest to the distal end of the robotic drive 24. The most proximal drive module is the drive module 32d positioned furthest from the distal end of the robotic drive 24 along the negative X axis. The relative position of drive modules is determined by their relative location to the distal end of the robotic drive. For example, drive module 32b is distal to drive module 32c. Referring to FIG. 3, the portions of cassette 66a and drive module 68a are defined by their relative location to the distal end of the robotic drive. For example, the distal end of cassette 66a is the portion of the cassette that is closest to the distal end of the robotic drive and the proximal end of cassette 66a is the portion of the cassette that is furthest from the distal end of the robotic drive along the negative X axis when the cassette is in-use position on drive module 68a. Stated in another way, the distal end of cassette 66a is the portion of the cassette through which an EMD is closest to the path leading to a patient in the in-use position.

Longitudinal axis: The term longitudinal axis of a member (for example, an EMD or other element in the catheter-based procedure system) is the line or axis along the length of the member that passes through the center of the transverse cross section of the member in the direction from a proximal portion of the member to a distal portion of the member. For example, the longitudinal axis of a guidewire is the central axis in the direction from a proximal portion of the guidewire toward a distal portion of the guidewire even though the guidewire may be non-linear in the relevant portion.

Axial Movement: The term axial movement of a member refers to translation of the member along the longitudinal axis of the member. When the distal end of an EMD is axially moved in a distal direction along its longitudinal axis into or further into the patient, the EMD is being advanced. When the distal end of an EMD is axially moved in a proximal direction along its longitudinal axis out of or further out of the patient, the EMD is being withdrawn.

Rotational Movement: The term rotational movement of a member refers to the change in angular orientation of the member about the local longitudinal axis of the member. Rotational movement of an EMD corresponds to clockwise or counterclockwise rotation of the EMD about its longitudinal axis due to an applied torque.

Axial and Lateral Insertion: The term axial insertion refers to inserting a first member into a second member along the longitudinal axis of the second member. An EMD that is axially loaded in a collet is axially inserted in the collet. An example of axial insertion could be referred to as back loading a catheter on the proximal end of a guidewire. The term lateral insertion refers to inserting a first member into a second member along a direction in a plane perpendicular to the longitudinal axis of the second member. This can also be referred to as radial loading or side loading. Stated another way, lateral insertion refers to inserting a first member into a second member along a direction that is parallel to the radius and perpendicular to the longitudinal axis of the second member.

Pinch/Unpinch: The term pinch refers to releasably fixing an EMD to a member such that the EMD and member move together when the member moves. The term unpinch refers to releasing the EMD from a member such that the EMD and member move independently when the member moves.

Clamp/Unclamp: The term clamp refers to releasably fixing an EMD to a member such that the EMD's movement is constrained with respect to the member. The member can be fixed with respect to a global coordinate system or with respect to a local coordinate system. The term unclamp refers to releasing the EMD from the member such that the EMD can move independently.

Grip/Ungrip: The term grip refers to the application of a force or torque to an EMD by a drive mechanism that causes motion of the EMD without slip in at least one degree of freedom. The term ungrip refers to the release of the application of the force or torque to the EMD by a drive mechanism such that the position of the EMD is no longer constrained. In one example an EMD is gripped between two tires rotates about its longitudinal axis when the tires move longitudinally relative to one another. The rotational movement of the EMD is different than the movement of the two tires. The position of an EMD that is gripped is constrained by the drive mechanism.

Buckling: The term buckling refers to the tendency of a flexible EMD when under axial compression to bend away from the longitudinal axis or intended path along which it is being advanced. In one embodiment axial compression occurs in response to resistance from being navigated in the vasculature. The distance an EMD may be driven along its longitudinal axis without support before the EMD buckles is referred to herein as the device buckling distance. The device buckling distance is a function of the device's stiffness, geometry (including but not limited to diameter), and force being applied to the EMD. Buckling may cause the EMD to form an arcuate portion different than the intended path. Kinking is a case of buckling in which deformation of the EMD is non-elastic resulting in a permanent set.

Homing: The term homing refers to moving a member to a defined position. An example of a defined position is a reference position. Another example of a defined position is an initial position. The term home refers to the defined position. It is normally used as a reference for subsequent linear or rotational positions.

Up/Down; Front/Rear; Inwardly/Outwardly: The terms top, up, and upper refer to the general direction away from the direction of gravity and the terms bottom, down, and lower refer to the general direction in the direction of gravity. The term front refers to the side of the robotic drive that faces a bedside user and away from the positioning system, such as the articulating arm. The term rear refers to the side of the robotic drive that is closest to the positioning system, such as the articulating arm. The term inwardly refers to the inner portion of a feature. The term outwardly refers to the outer portion of a feature.

Stage: The term stage refers to a member, feature, or device that is used to couple a device module to the robotic drive. For example, the stage may be used to couple the device module to a rail or linear member of the robotic drive.

Drive Module: The term drive module generally refers to the part (e.g., the capital part) of the robotic drive system that normally contains one or more motors with drive couplers that interface with the cassette.

Device Module: The term device module refers to the combination of a drive module and a cassette.

Cassette The term cassette generally refers to the part (non-capital, consumable or sterilizable unit) of the robotic drive system that normally is the sterile interface between a drive module and at least one EMD (directly) or through a device adapter (indirectly).

Collet: The term collet refers to a device that can releasably fix a portion of an EMD. The term fixed here means no intentional relative movement of the collet and EMD during operation. In one embodiment the collet includes at least two members that move rotationally relative to each other to releasably fix the EMD to at least one of the two members. In one embodiment the collet includes at least two members that move axially (along a longitudinal axis) relative to each other to releasably fix the EMD to at least one of the two members. In one embodiment the collet includes at least two members that move rotationally and axially relative to each other to releasably fix the EMD to at least one of the two members.

Fixed: The term fixed means no intentional relative movement of a first member with respect to a second member during operation.

On-Device Adapter: The term on-device adapter refers to a sterile apparatus capable of releasably pinching an EMD to provide a driving interface. The on-device adapter is also known as an end-effector or EMD capturing device. In one non-limiting embodiment the on-device adapter is a collet that is operatively controlled robotically to rotate the EMD about its longitudinal axis, to pinch and/or unpinch the EMD to the collet, and/or to translate the EMD along its longitudinal axis. In one embodiment the on-device adapter is a hub-drive mechanism such as a driven gear located on the hub of an EMD.

Tandem Drive: The term tandem drive refers to a drive unit or subsystem within the robotic drive containing two or more EMD drive modules, capable of manipulating one or more EMDs.

EMD: The term elongated medical device (EMD) refers to, but is not limited to, catheters (e.g., guide catheters, microcatheters, balloon/stent catheters), wire-based devices (e.g., guidewires, embolization coils, stent retrievers, etc.), and medical devices comprising any combination of these. In one example a wire-based EMD includes but is not limited to guidewires, microwires, a proximal pusher for embolization coils, stent retrievers, self-expanding stents, and flow divertors. Typically wire-based EMD's do not have a hub or handle at its proximal terminal end. In one embodiment the EMD is a catheter having a hub at a proximal end of the catheter and a flexible shaft extending from the hub toward the distal end of the catheter, wherein the shaft is more flexible than the hub. In one embodiment the catheter includes an intermediary portion that transitions between the hub and the shaft that has an intermediate flexibility that is less rigid than the hub and more rigid than the shaft. In one embodiment the intermediary portion is a strain relief.

Hub (Proximal) Driving: The term hub driving or proximal driving refers to holding on to and manipulating an EMD from a proximal position (e.g., a geared adapter on a catheter hub). In one embodiment, hub driving refers to imparting a force or torque to the hub of a catheter to translate and/or rotate the catheter. Hub driving may cause the EMD to buckle and thus hub driving often requires anti-buckling features. For devices that do not have hubs or other interfaces (e.g., a guidewire), device adapters may be added to the device to act as an interface for the device module. In one embodiment, an EMD does not include any mechanism to manipulate features within the catheter such as wires that extend from the handle to the distal end of the catheter to deflect the distal end of the catheter.

Shaft (Distal) Driving: The term shaft (distal) driving refers to holding on to and manipulating an EMD along its shaft. In one example the on-device adapter is normally placed just proximal of the hub or Y-connector the device is inserted into. If the location of the on-device adapter is at the proximity of an insertion point (to the body or another catheter or valve), shaft driving does not typically require anti-buckling features. (It may include anti-buckling features to improve drive capability.)

Sterilizable Unit: The term sterilizable unit refers to an apparatus that is capable of being sterilized (free from pathogenic microorganisms). This includes, but is not limited to, a cassette, consumable unit, drape, device adapter, and sterilizable drive modules/units (which may include electromechanical components). Sterilizable Units may come into contact with the patient, other sterile devices, or anything else placed within the sterile field of a medical procedure.

Sterile Interface: The term sterile interface refers to an interface or boundary between a sterile and non-sterile unit. For example, a cassette may be a sterile interface between the robotic drive and at least one EMD.

Reset The term reset means repositioning a drive mechanism from a first position to a second position to allow for continued rotational and/or axial movement of an EMD. During reset, the EMD is not actively being moved by the drive mechanism. In one embodiment the EMD is released by the drive mechanism prior to repositioning the drive mechanism. In one embodiment a clamp fixes the location of the EMD during repositioning of the drive mechanism.

Continuous Motion: The term continuous motion refers to motion that does not require a reset and is uninterrupted.

Discrete Motion: The term discrete motion refers to motion that requires a reset and is interrupted.

Consumable: The term consumable refers to a sterilizable unit that normally has a single use in a medical procedure. The unit could be a reusable consumable through a re-sterilization process for use in another medical procedure.

Device Support: The term device support refers to a member, feature, or device that prevents an EMD from buckling.

Double Gear: The term double-gear refers to two independently driven gears operatively connected to two different portions of a device. Each of the two gears may be identical or different design. The term gear may be a bevel gear, spiral bevel gear, spur gear, miter gear, worm gear, helical gear, rack and pinon, screw gear, internal gear such as a sun gear, involute spline shafts and bushing, or any other type of gears known in the art. In one example, double-gear also includes devices in which any drive connection is maintained by two different portions of a device, including but not limited to a belt, friction engagement or other couplers known in the art.

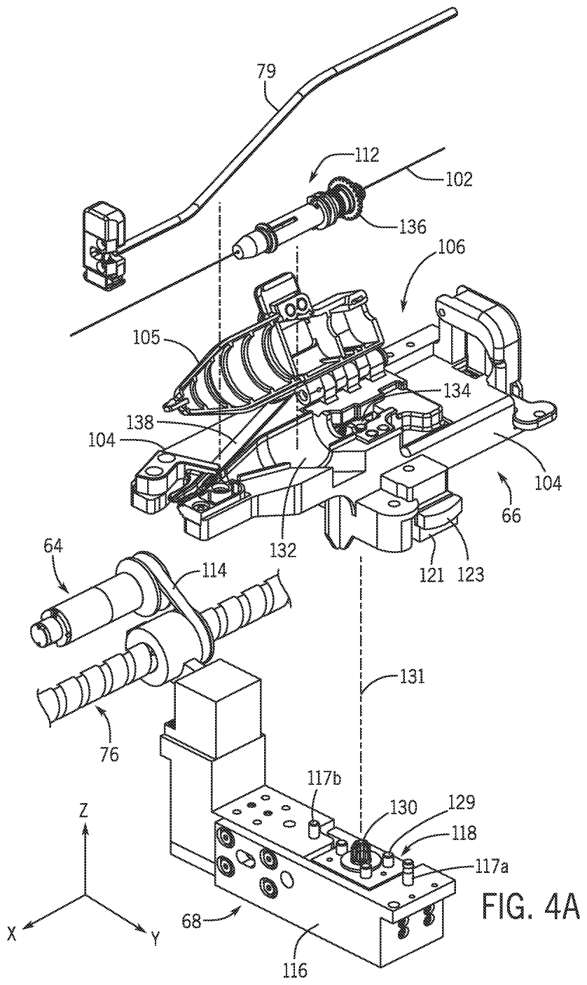

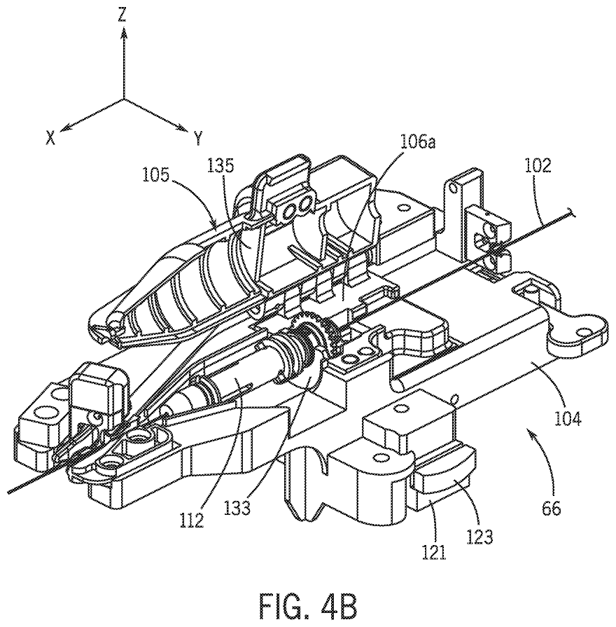

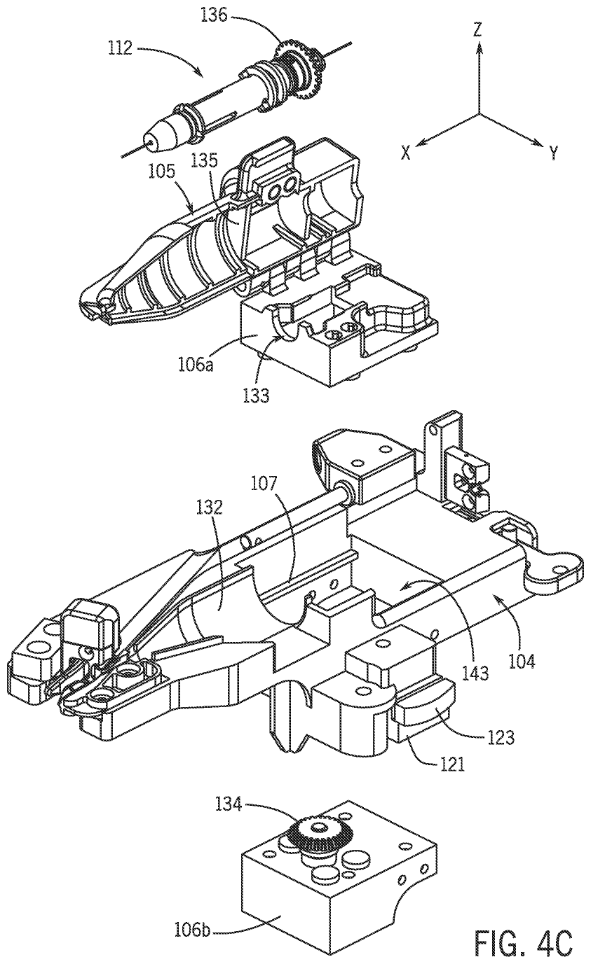

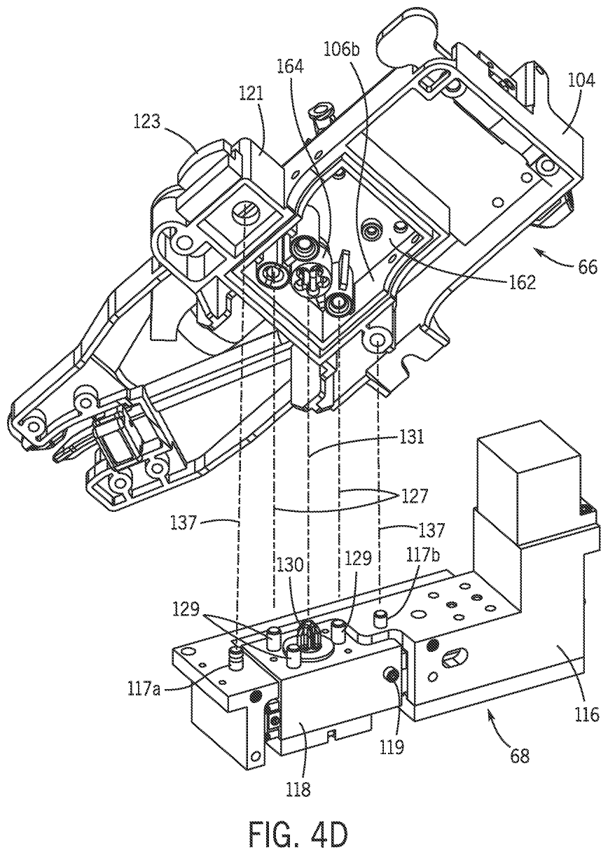

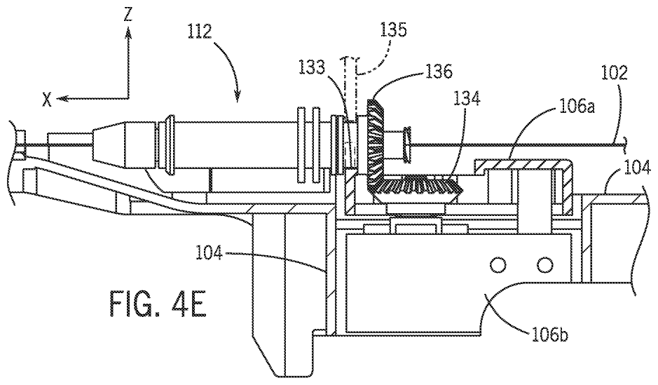

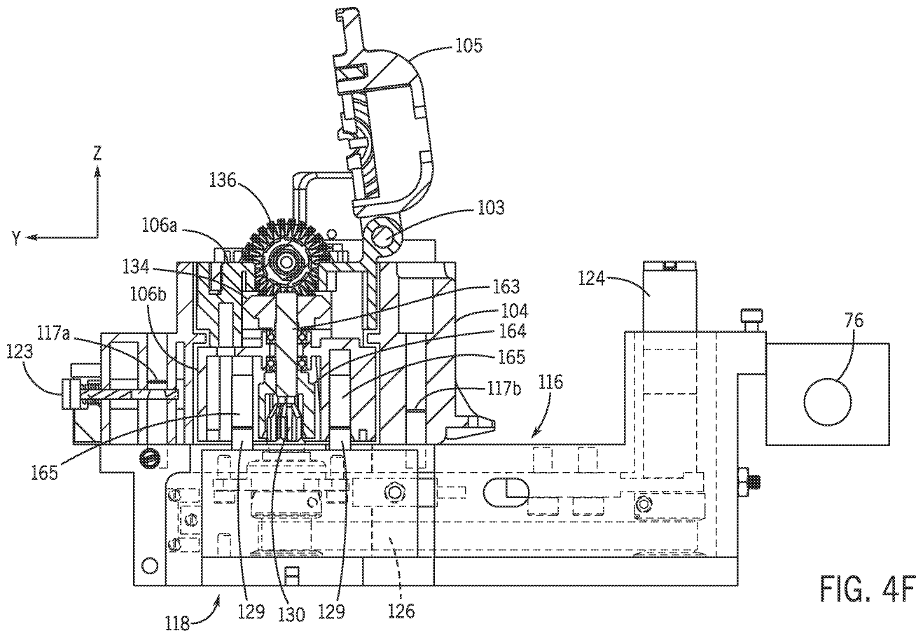

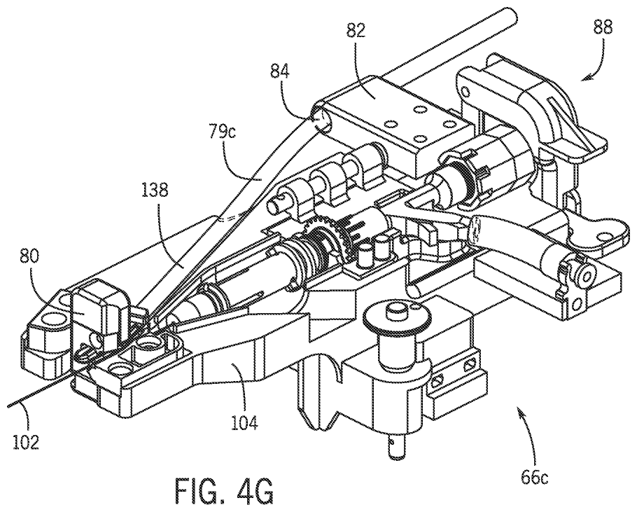

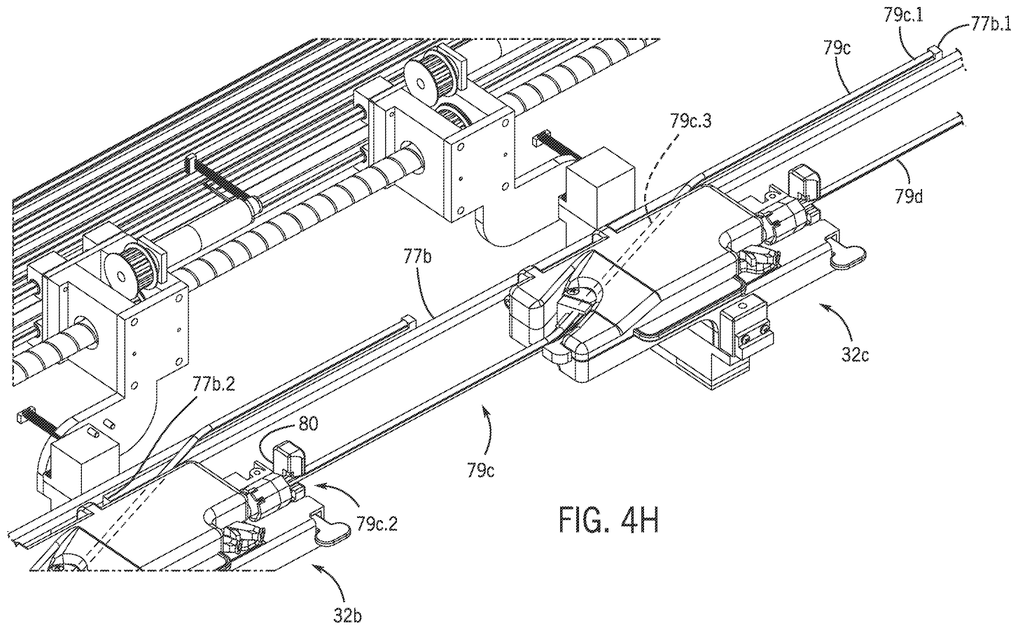

Referring to FIG. 3 and FIG. 4A an EMD drive system includes an on-device adapter 112 which in one embodiment includes a collet that is removably fixed to an EMD 102. Collet 112 is a device that releasably fixes a shaft portion of EMD 102 thereto. As described in more detail herein collet 112 pinches the shaft of EMD 102 such that rotation and/or translation of the entire collet 112 about or along its longitudinal axis results in the same rotation and/or translation of the portion of the shaft of EMD 102 that is pinched. In one embodiment collet 112 may be a single molded component having a body defining an internal pathway through which a portion of the shaft of the EMD 102 may be fixed. As described herein the shaft of the EMD 102 is positioned in the internal pathway of the collet and pinched therein. The shaft of the EMD 102 may be radially loaded or axially-loaded into the internal pathway of the collet. Radially loaded may also be referred to as side-loaded or laterally loaded since the shaft of the EMD is loaded into the collet 112 through a longitudinal side of the collet body (that is the side of the collet body extending from a proximal end to the distal end of the collet body). Radially loading, side loading or laterally loading is in contrast to axially loading in which a shaft portion is loaded into the internal pathway by first inserting a free end of the shaft into a proximal or distal opening in the collet's internal pathway.

In one embodiment the collet 112 includes at least two members that move relative to each other to releasably fix the shaft portion of the EMD to at least one of the two members. In one embodiment the two members operating together provide a mechanical advantage that increases the torque and/or force that may be transmitted from the collet body to the shaft of the EMD without the shaft of the EMD moving relative to the collet body. The pinch force on the EMD using a collet can be greater than the force required to actuate the pinch. When the shaft of the EMD is pinched it is fixed such that there is relative movement of the collet and EMD during acceptable operation parameters of an EMD procedure.

EMD 102 is fixed to the collet 112 and radially loaded into a robotic drive also referred to herein as a device module 32 such as an EMD drive. An EMD support 79 is removably applied to EMD 102 from a non-axial direction. Robotic drive 32 is operatively coupled to collet 112 to translate and/or rotate collet 112 and EMD 102. In one embodiment EMD 102 is removably and releasably loaded into the robotic drive 32.

In one embodiment collet 112 is in robotic drive 32 when EMD 102 is radially loaded into robotic drive 32. In one embodiment collet 112 is removably inserted into robotic drive 32 with EMD 102 fixed to collet 112.

In one embodiment EMD support 79 limits buckling and prevents kinking of EMD 102 along its length as EMD 102 is being translated and/or rotated.

In one embodiment a robotic system includes a robotic drive 32 or device module includes a drive module 68 or base having a drive coupler 130, and a cassette 66 removably secured to the drive module 68. Collet 112 in cassette 66 is removably fixed to EMD 102. Collet 112 has a driven member 136 operatively coupled to drive coupler 130. The robotic drive 32 includes a motor or actuator operatively coupled to collet 112 to move collet 112. In one embodiment cassette 66 is removably secured to base 68 by directly connecting cassette 66 to base 68. In one embodiment cassette is 66 is removably secured to base 68 indirectly in which an intermediate member is positioned between cassette 66 and base 68.

EMD 102 may be radial loaded or axially loaded into collet 112 prior to collet 112 being positioned within cassette 66 such that EMD 102 and collet 112 are loaded into cassette 66 together. EMD 102 may be radial loaded or axially loaded into collet 112 or when collet 112 is already positioned within cassette 66.

In one embodiment EMD 102 is removably received in collet 112 in a radial direction and collet 112 is removably received and positioned in cassette 66. As described herein collet 112 may have a slot extending from an outer periphery of a collet body extending to its internal pathway. A portion of EMD 102 such as a shaft portion may be inserted into the pathway through the slot in a radial direction. The shaft portion of EMD 102 is a portion of the EMD 102 intermediate a proximal end of EMD 102 and a distal end of EMD 102. Radial loading of the shaft portion of EMD 102 into the collet occurs while the proximal end of EMD 102 and the distal end of EMD 102 remain outside of the collet and pathway. Stated another way shaft portion of EMD 102 is loaded in a direction generally perpendicular to a longitudinal axis of collet 112.

In one embodiment EMD 102 is removably received in collet 112 in an axial direction and collet 112 is removably received in cassette 66. In this embodiment one of the distal end or proximal end of EMD 102 is inserted into a distal opening or proximal opening collet 112 and moved along the longitudinal axis of collet 112 until the distal end or proximal end of EMD exits the other of the distal end or proximal end of collet.

In one embodiment EMD 102 is removably received in collet 112 in a radial direction and collet 112 is non-removably positioned within cassette 66. In one embodiment EMD 102 is removably received in collet 112 in an axial direction and collet 112 is non-removably positioned within cassette 66. In one embodiment collet 112 includes a locating feature 408 that is located within cassette 66 with a locating feature 133 that allows for radial loading as well as rotation of the collet within the cassette 66. In one embodiment collet 112 also includes a distal end that that is located within a locating feature in cassette 66.

Referring to FIG. 4F in one embodiment a motor 124 is positioned within base 68 operatively coupled to drive coupler 130. Drive coupler 130 extends into cassette 66, when cassette 66 is secured to base 68. In one embodiment the motor is located in cassette 66. In one embodiment the motor is located outside of the base 68 but operatively connected to the drive coupler 130 in the base 68.

In one embodiment robotic system includes a clamp releasably clamping a shaft portion of the EMD independent of the collet. In one embodiment the clamp includes at least one tire.

As discussed in more detail herein in one embodiment moving collet 112 rotates the collet and EMD. In one embodiment EMD 102 is selectively rotated in a clockwise and counterclockwise direction about a longitudinal axis of EMD 102.

As discussed in more detail herein in one embodiment moving collet 112 selectively pinches and unpinches the EMD within the collet. In one embodiment as discussed in detail herein moving collet 112 includes moving only one or more parts of collet 112 and not the entire collet to pinch and unpinch the EMD.

As discussed in more detail herein in one embodiment moving collet 112 selectively translates the collet and EMD in a first direction and opposite second direction along a longitudinal axis of the EMD.

As discussed in more detail herein in one embodiment moving collet 112 includes rotating the collet and EMD, translating the collet and EMD and selectively pinching and unpinching the EMD within the collet.

Referring to FIGS. 3, 4G and 4H robotic system 24 includes a plurality of device modules 32a-32d. In one embodiment there are two or more separate device modules. FIG. 3 illustrates a system with four device modules 32. In one embodiment the modules are identical and in one embodiment each device module is different or some modules are identical and some are different. FIG. 3 as discussed above illustrates a system with four device modules 32. Each EMD device support 79a-79d includes a proximal end and a distal end terminating in a distal connector 80. By way of example, referring to FIG. 4H, device module 32c has an EMD device support 79c that has a proximal end 79c.1 and an opposing distal end connector 79c.2. Proximal end 79c.1 of EMD device support 79c is secured to a proximal end 77b.1 of arm 77b. Arm 77b has a distal end 77b.2 that is secured to device module 32b that is distal to device module 32c. The terminal end 77b.2 of EMD drive support device 77b is secured to the proximal end of device module 32b so that the terminal end 77b.2 cannot be moved distal to the distal terminal end of device module 32b. In operation distal end connector 80c is removably connected to a proximal end connector 88b on device module 32b. In one embodiment EMD supports 79a-79d include a flexible tube having a longitudinal slit permitting an EMD to be inserted into and removed from respective EMD device supports 79a-79d. In one embodiment EMD supports 79a-79d operates as the flexible track described in US Published Application No. US 2016/0271368 entitled Guide Catheter Control Flexible Track owned by the same applicant as the instant application. Arm 77b moves linearly with drive module 32b and accordingly, in one mode proximal end 77c.1 and distal end 77c.2 moves with drive module 32b relative to drive module 32c. EMD device support 79c is removably applied to the EMD 102 being manipulated by device module 32c in a non-axial direction. The EMD 102 being manipulated by device module 32c enters and exits support 79c via the longitudinal slit extending from the outer periphery of the EMD device support to the inner lumen of the EMD support. In one embodiment EMD device support is a telescoping member as discussed further herein in which the EMD may be axially loaded or non-axially loaded within the EMD device support to provide anti-buckling support. Referring to FIG. 3 each drive module 32a-32d independently manipulates a different device. Each EMD device support 79a-79d allows each device to be translated a greater distance between two adjacent devices than could be translated without an EMD Support. Without EMD device supports, the distance a device could be translated would be less than the buckling length of the device. Accordingly, the system would need to reset the drive each time the EMD is moved the buckling length. The EMD supports allow for non-reset during use of certain devices in conjunction with each other and/or procedures. Stated another way other words EMD device support allows the collet not to be reset when using certain devices. In one embodiment EMD Supports allow for fewer resets of the collet than would otherwise be necessary without the EMD supports. Referring to FIG. 4G device support 79 is guided through cassette 66c via a channel 138 and a proximal support member 82 via a channel 84 that extends therethrough.

EMD 102 may be pinched by on-device adapter and/or collet 112 by manually manipulating collet 112 and then the collet and EMD are robotically rotated and translated. In one embodiment EMD 102 is robotically pinched and unpinched by collet 112 as well as robotically rotated and translated by rotating and translating collet 112.

A number of robotic EMD drive systems are described herein. Additionally, a number of collet designs are also described herein. The specific collet designs described herein, and collet designs known in the art may be used in the various EMD drive systems described. Collets as described herein may also referred to in the art as a pin vise, chuck, bushing, or guidewire torquer.

Referring to FIGS. 1, 4A and 4D device module 32 includes a drive module 68 including a drive module base component 116 and a load-sensed component 118. An EMD 102 is removably coupled to an isolated component 106. The isolated component 106 is isolated from an external load other than an actual load acting on the EMD 102. The isolated component 106 is removably coupled to the load-sensed component 118. A load sensor 120 that is secured to the drive module base component 116 and the load-sensed component 118 senses the actual load acting on the EMD 102.

In one embodiment load sensor 120 is the sole support of the load-sensed component 118 in at least one direction of load measurement. In one embodiment cassette housing 104 and isolated component 106 are internally connected so they form one component. In one embodiment a flexible membrane 108 connects cassette housing 104 and isolated component 106, where flexible membrane 108 applies negligible forces in the X-direction (device direction) to the isolated component 106. In one embodiment, flexible membrane 108 is not a physical membrane and represents the cassette interaction.

Referring to FIGS. 4A and 4B in one embodiment the apparatus includes a cassette 66 that is comprised of a cassette housing 104 removably attached to the drive module base component 116 and a cassette cover 105.

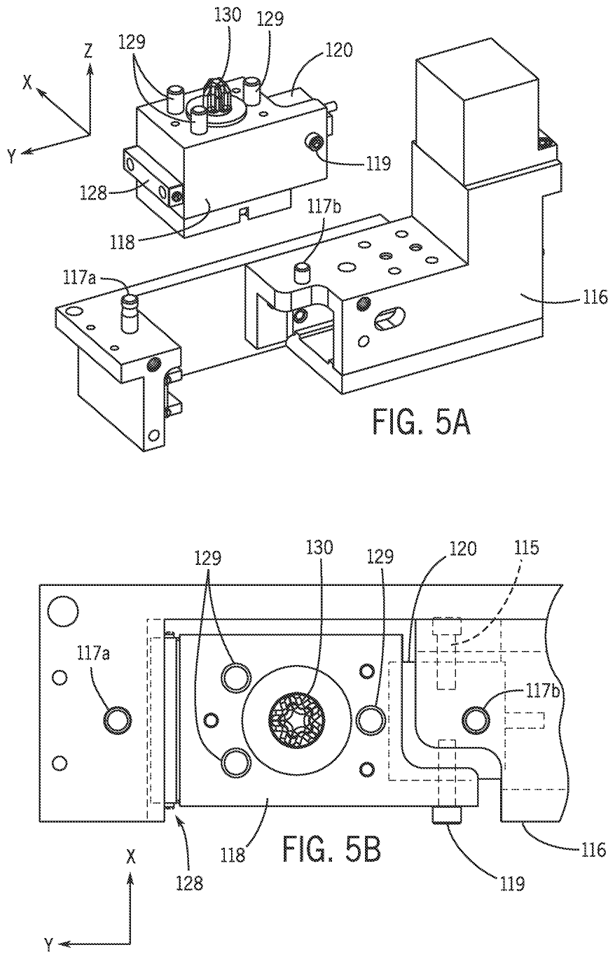

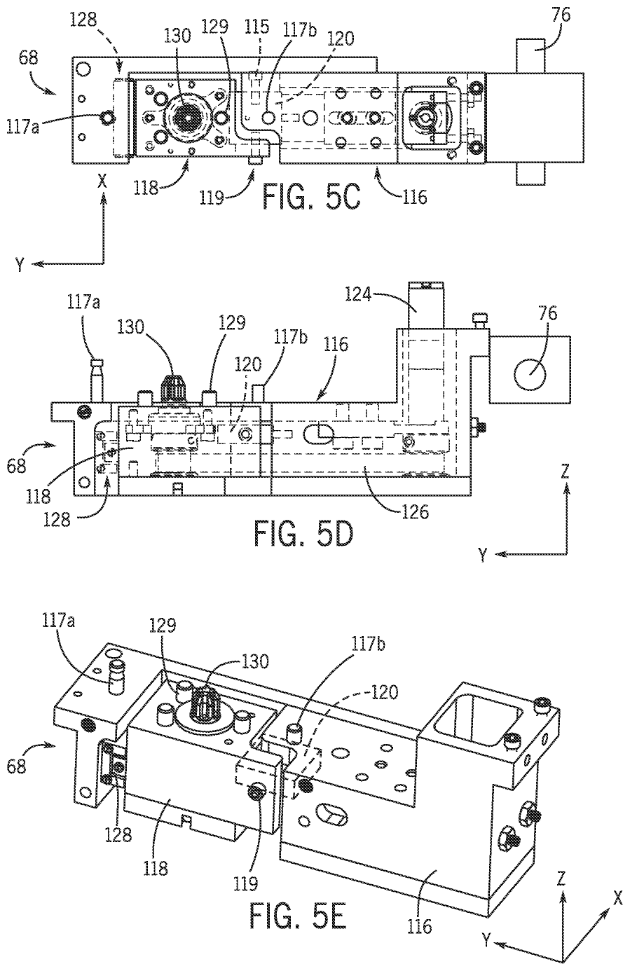

Referring to FIGS. 5C-5E in one embodiment the drive module base component 116 includes the load-sensed component 118 and load sensor 120. Drive module 68 includes drive module base component 116 and load-sensed component 118 as separate parts that are connected by load sensor 120 that is located between drive module base component 116 and load-sensed component 118. Bearing 128 of load-sensed component 118 supports the load-sensed component in at least one off-axis (non-measured) direction.

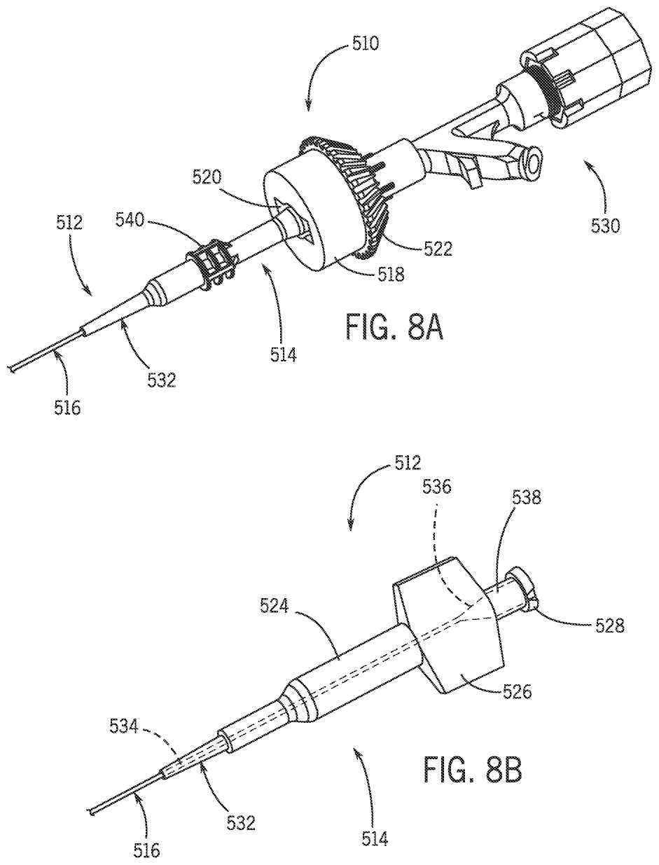

Referring to FIGS. 8A and 8B, in one embodiment EMD on-device adapter 112 is connected to a catheter 140. On-device adapter 112 includes an integrally connected driven bevel gear 136 that can be removably connected to a Y-connector shown with hub 142 that can be removably connected to a hemostasis valve on the proximal end. One embodiment of EMD on-device adapter 112 includes a catheter 140 removably connected to a driven bevel gear 136. Catheter 140 includes a catheter hub 139 and a catheter shaft 141 that are integrally connected. In one embodiment catheter hub 139 is not a handle that includes mechanisms that manipulate a feature or portion of the catheter. In one embodiment an EMD includes a handle with mechanisms to manipulate features within the catheter such as wires that extend from the handle to the distal end of the catheter to steer or deflect the distal end of the catheter. In contrast the hub is the rigid portion of the EMD at the proximal end that does not include mechanisms to manipulate features within the catheter.

Referring to FIGS. 4B and 4C the isolated component 106 is positioned within and separate from the cassette housing 104 in at least one direction when the isolated component 106 is connected to the load-sensed component 118. Isolated component 106 includes a first component 106a and a second component 106b attached thereto. Referring to FIGS. 4A-4C the first component 106a is placed within a recess 143 of the cassette housing 104 in a first direction that is defined as the direction toward the drive module 68 when the cassette 66 is in the in-use position secured to the drive module 68. The second component 106b is placed within the recess 143 from a direction away from the load-sensed component 118 toward the first component 106a. Stated another way referring to FIG. 4C first component 106a is placed in recess 143 in a −z axis direction from above the cassette housing 104 and the second component 106b is placed in recess 143 in a +z axis direction from below the cassette housing 104.

Referring to FIGS. 4C and 4F the first component 106a and second component 106b are secured to one another. Cassette housing 104 includes two longitudinally oriented and spaced parallel rails 107 located within the recess 143. Rails 107 are also referred to as linear guides herein. Rails 107 are substantially parallel to one another and spaced from one another. The first component 106a is located on the top surface of rails 107 closest to the top surface of the cassette housing 104 and the second component 106b is located on the bottom surface of rails 107 closest to the load-sensed component 118. Note that although the direction of assembly of first component 106a and second component 106b of the isolated component 106 is described in relation to the in-use position, the first and second components of the isolated component 106 are installed away from the drive module 68. Stated another way, the first component 106a of the isolated component 106 is inserted into the recess 143 in a direction from a top surface of the cassette 66 toward the bottom surface of the cassette 66 in a direction generally perpendicular to the longitudinal axis of the cassette housing 104.

In one embodiment a mechanical fastener or plurality of fasteners secure the first component 106a to the second component 106b of the isolated component 106. In one embodiment the first component 106a and second component 106b are secured together using magnets. In one embodiment the first component 106a and second component 106b of the isolated component 106 are secured with an adhesive. In one embodiment the first component 106a and second component 106b are releasably secured to one another without the use of tools. In one embodiment the first component 106a and second component 106b are non-releasably secured to one another.

Referring to FIG. 4F in an in-use position where the second component 106b of the isolated component 106 is releasably secured to the load-sensed component 118, the first component 106a and second component 106b are spaced from the rails 107 of the cassette housing 104 such that the first component 106a and second component 106b are in a non-contact relationship with cassette housing 104.

In one embodiment the on-device adapter 112 is spaced from and in non-contact with the cassette housing 104 when the on-device adapter 112 is coupled to the load-sensed component 118. In one embodiment the isolated component 106 is separate from the cassette housing 104 in all directions. In one embodiment the isolated component 106 is separate from and in a non-contact relationship with the cassette housing 104.

Referring to FIGS. 4B, and 4C, in one embodiment the cassette 66 includes a cassette cover 105 pivotably coupled by hinge 103 to the isolated component 106 separate and in non-contact with the cassette housing 104. In one embodiment the cassette cover 105 is pivotably coupled by hinge 103 to the first component 106a of the isolated component 106. In one embodiment the cassette cover 105 is connected to the first component 106a of the isolated component 106 by other means, such as snap fits.

Referring to FIGS. 1 and 4C, in one embodiment the drive module 68 moves the EMD 102 in a first direction, the isolated component 106 being separate from the cassette housing 104 in the first direction. In one embodiment the drive module 68 moves the EMD 102 in a second direction, the isolated component 106 being separate from the cassette housing 104 in the first direction and the second direction.

Referring to FIG. 4D in one embodiment second component 106b of the isolated component 106 is releasably secured to the load-sensed component 118 with fasteners. In one embodiment the fasteners include a quick release mechanism that can releasably secure the second component 106b of the isolated component 106 to the load-sensed component 118. In one embodiment the fasteners are magnets.

Referring to FIGS. 5A-5E-sensed component 118 is located within the drive module base component 116 and secured to the drive module base component 116 with a load sensor 120. In one embodiment load sensor 120 includes a first portion secured to drive module base component 116 with a first fastener 115 and a second portion secured to load-sensed component 118 with a second fastener 119. In one embodiment the first portion of the load sensor 120 is different and distinct from the second portion of the load sensor 120. In one embodiment first fastener 115 and second fastener 119 are bolts. In one embodiment first fastener 115 and second fastener 119 are mechanical fastening components known in the art for ensuring mechanical connection. In one embodiment first fastener 115 and second fastener 119 are replaced with adhesive means for ensuring mechanical connection. In one embodiment first fastener 115 and second fastener 119 are magnets.

Referring to FIG. 5A in one embodiment drive module base component 116 includes a recess that receives load-sensed component 118. In one embodiment drive module base component 116 further defines a cavity extending from recess that receives a portion of load sensor 120.

Referring to FIGS. 4B and 4D in one embodiment cassette housing 104 is releasably connected to drive module base component 116 via a quick-release mechanism 121. In one embodiment quick-release mechanism 121 includes a spring-biased member in the cassette housing 104 that is activated by a latch release 123 that releasably engages with a quick release locking pin 117a secured to the drive module base component 116. In one embodiment an alignment pin 117b secured to the drive module base component 116 aligns the cassette housing 104 relative to the drive module base component 116.

Referring to FIGS. 4C and 4F, isolated component 106 is contained inside cassette housing 104 by attaching first component 106a to second component 106b of isolated component 106 about rails 107 in cassette housing 104. In the in-use position, isolated component 106 is not in contact with rails 107. In this way, load interaction due to an external force and/or external torque acting on EMD 102 occurs with one component in the cassette 66.

Cassette housing 104 includes a cradle 132 configured to receive EMD on-device adapter 112 with EMD 102. A cassette bevel gear 134 in cassette housing 104 can freely rotate with respect to cassette housing 104 about an axis aligned with a coupler axis 131 about which coupler 130 of drive module 68 rotates. In the assembled device module 32, cassette 66 is positioned on mounting surface of drive module 68 such that cassette bevel gear 134 receives coupler 130 along coupler axis 131 in such a way that it is free to engage and disengage along coupler axis 131 and integrally connected (not free) about coupler axis 131 such that rotation of coupler 130 corresponds equally to rotation of cassette bevel gear 134. In other words, if coupler 130 rotates clockwise at a given speed, then cassette bevel gear 134 rotates clockwise at the same given speed, and if coupler 130 rotates counterclockwise at a given speed, then cassette bevel gear 134 rotates counterclockwise at the same given speed.

Referring to FIGS. 1, 3, and 4 an EMD drive system includes an on-device adapter 112 removably fixed to a shaft of an EMD 102. The on-device adapter 112 is received in a cassette 66 removably secured to a drive module 68. The drive module 68 is operatively coupled to the on-device adapter 112 to move the on-device adapter 112 and EMD 102 together.

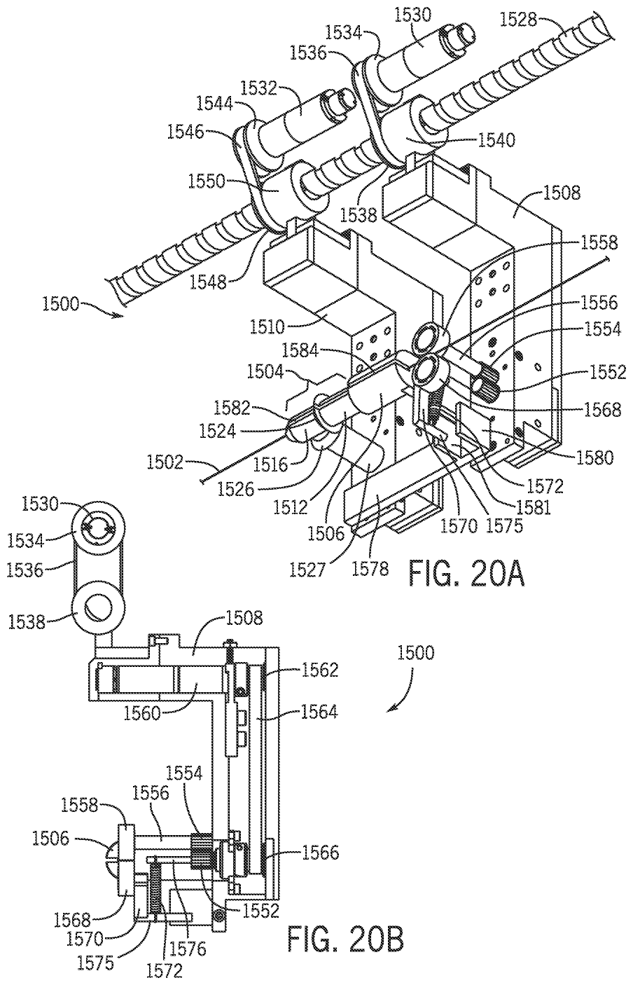

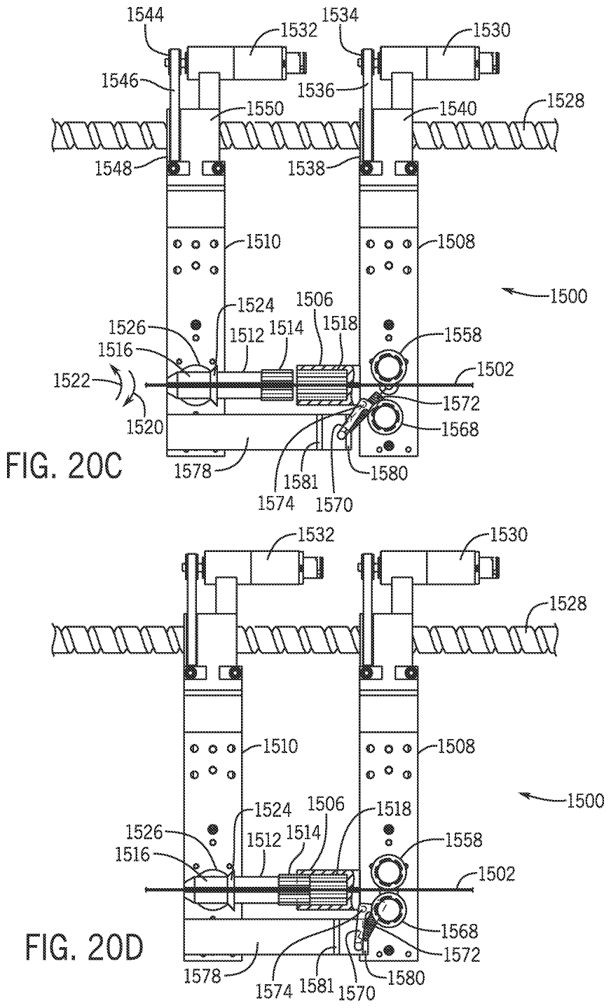

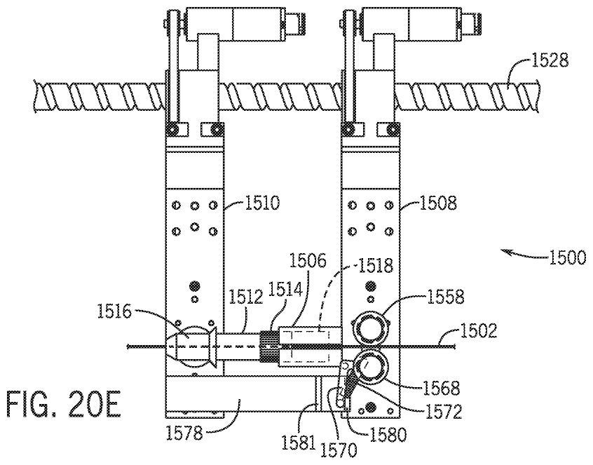

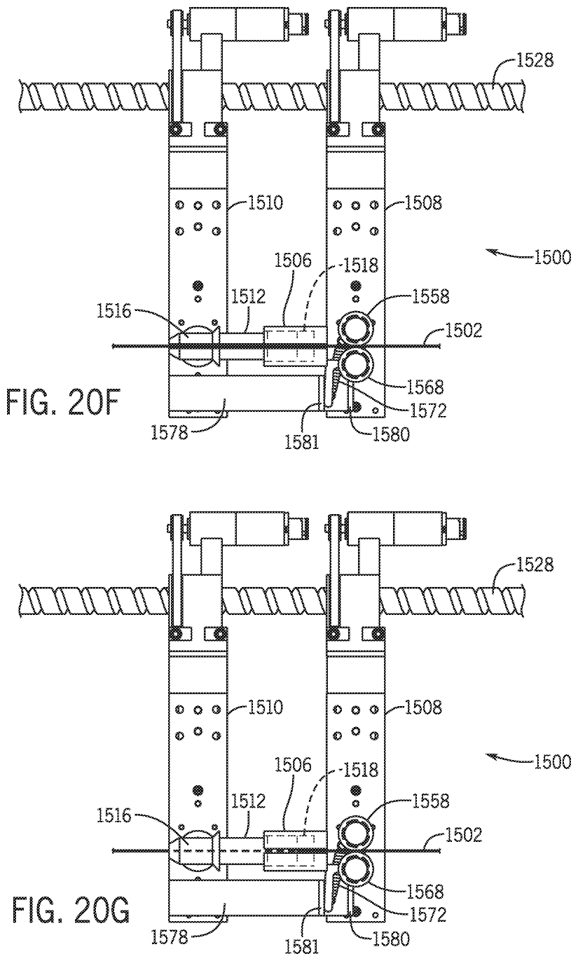

In one embodiment the on-device adapter 112 is moved in translation. Referring to FIG. 3 drive module 68 is moved along the X axis to translate the cassette 68, on-device adapter 112 and EMD 102 together. In one embodiment translation along the x-axis is co-axial to the longitudinal axis of the on-device adapter 112, the longitudinal axis of the cassette and the longitudinal axis of EMD 102. Referring to FIG. 20A drive module includes a reset function that moves the on-device adapter and EMD in translation. Moving in translation moves the elements noted above along the longitudinal axis of the cassette and on-device adapter in the distal and proximal directions.

In one embodiment the on-device adapter is moved in rotation about the longitudinal axis of the on-device adaptor.

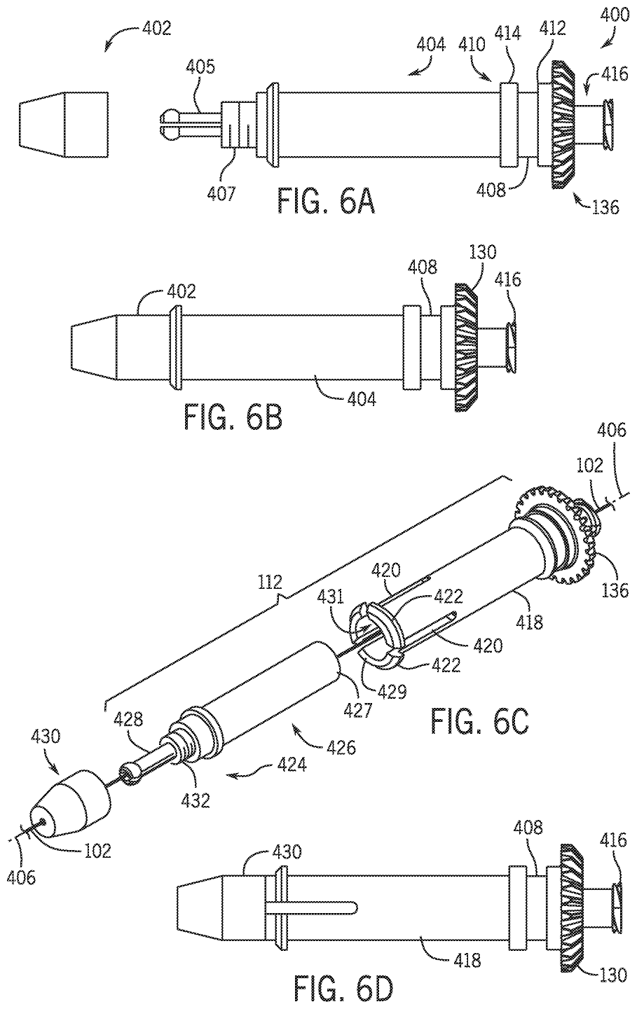

In one embodiment the on-device adapter 112 includes a collet. Collet can include a variety of collet designs included but not limited to the collets discussed herein. See FIGS. 6A, 6B, 9A-9I, and 10A-11E.

Referring to FIGS. 6A and 6B in one embodiment collet 400 includes a first member 402 moving along and/or about a longitudinal axis 406 of the second member 404 to pinch the shaft of EMD 102 within a third member 405. In one embodiment second member 404 is generally cylindrical. However, second member 404 may be other geometric shapes such as frustoconical with the first portion having a cross section closer to engagement portion 136 that is smaller than a second cross section of a second portion being closer to first member 402. In one embodiment first member 402 is referred to a nut, second member 404 is referred to as a collet body or sleeve and a third member 405 is referred to as a chuck. Nut 402 is fastened to body 404 to open and close chuck 405 to pinch and unpinch EMD 102. In one embodiment nut 402 is threadably engaged with body 404.

On-device adapter 112 includes an engagement portion 136 engaged with and driven by a drive member 134 in the cassette 66 to rotate on-device adapter 112. In one embodiment 136 engagement portion is a gear. However other engagement portions that are driven by drive members are contemplated.

In one embodiment on-device 112 adapter includes a surface 408 that is supported by a bearing member in the cassette.

In one embodiment the on-device 112 adapter includes a thrust bearing surface 410 preventing translational movement relative to a portion of cassette 66. In one embodiment the thrust bearing surface 410 includes a first portion 412 preventing translational movement in the distal direction and a second portion 414 preventing translation movement in the proximal direction. In one embodiment first portion 412 and second portion 414 form a groove therebetween defining surface 408 that is supported by a bearing member 133 in cassette 66.

In one embodiment the on-device adapter 112 includes a luer connector 416. In one embodiment luer connector 416 is covered by ISO 80369-7 standard incorporated herein by reference. In one embodiment luer connector 416 is configured to allow the on-device 112 adapter to be flushed with a cleaning fluid. Luer connector has a passage therethrough connected with a passage in the on-device adapter 112. In one embodiment the passage is in the luer connector 416 is co-axial and in fluid communication with the passageway in the on-device adapter. In one embodiment the passage in the on-device adapter 112 is the passage that receives the shaft of the EMD 102. In one embodiment luer connector 41 is a generic connector and in one embodiment it is a connector that falls within ISO 80369-7. In one embodiment luer connector is a luer lock.

Referring to FIGS. 6C and 6D on-device adapter 112 includes a holder 418 that has a engagement surface or gear 136 formed or attached thereto. Holder 418 has a plurality of slits 420 on a distal portion thereof extend to the distal end of holder 418 forming a plurality of fingers 422. Holder 418 has a channel that receives a proximal portion of a collet 424. In one embodiment collet 424 is an off the shelf torque device sold by Merit under the trademark Pin Vise. Collet 424 has a body proximal portion 426 having an outer diameter that is greater than the inner diameter at the distal end of the channel of holder 418. The proximal end of body 426 is placed within the channel of the holder 418 such that fingers 422 move outward thereby capturing collet 424 within holder 418 such that translation and/or rotation of holder 418 results in translation and/or rotation of collet 424. A second member 430 rotates about a threaded portion 432 of collet body portion 426 there by pinching a shaft of an EMD within split member portions 428. Slit member portions 428 move toward one another thereby pinching EMD 102 as the internal cone portion of second member 430 moves toward body portion 426 thereby engaging and moving split member portions 428 toward one another.

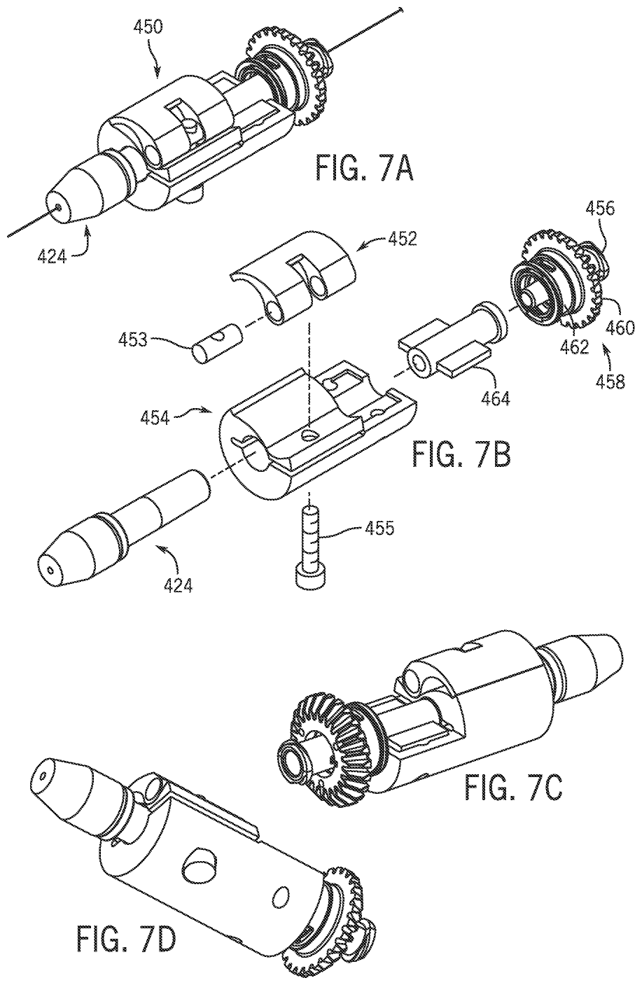

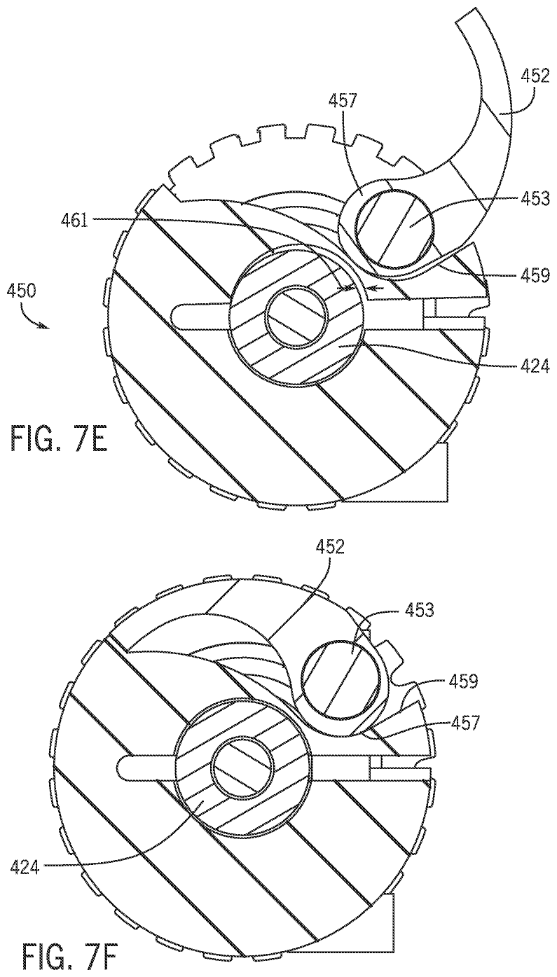

Referring to FIGS. 7A and 7B an on-device adapter 112 is an assembly that includes a quick clamp 450 engaging collet 424 as discussed above. However, it is contemplated that quick clamp 450 engages other collet designs. In one embodiment quick clamp 450 quickly connects and/or releases collet 424. Referring to FIGS. 7E and 7F lever 452 moves from a first unclamped position to a second clamped position to clamp the collet thereto. In one embodiment no additional tool is required to releasably engage the quick clamp onto the collet. Referring to FIGS. 7A and 7B quick clamp 450 includes a clamp body 454 defining a channel therethrough that receives a collet 424 such as a torquer described herein above. In one embodiment torquer 424 includes a proximal end 427 that is inserted into a distal opening 429 of the channel 431. A second portion 430 of the torquer that rotates relative to the body 426 acts to pinch and unpinch an EMD in a channel defined by the body and second portion. Referring to FIGS. 7E and 7F lever 452 pivotally attached to a clamp body 454 moves from a first open position to a second closed position in which the clamp body moves from an unclamped to a clamped position. Lever 452 includes a cam portion 457 that interacts with portion 459 on cam body 454. In the first open position a gap 461 exists between the outer surface of the collet body 454 and the surface of the clamp channel. Gap 461 allows the quick clamp 450 to secure a multitude of different commercially available collets with varying outer body diameters. As lever is pivoted from the open position to the closed position the gap 461 is eliminated there by clamping the collet body to the quick clamp such that translation and/or rotation of the quick clamp results in respective translation and/or rotation of the collet and EMD that is pinched in the collet. Gap 461 is eliminated as cam portion 457 interacts with surface 459 forcing body 454 to eliminate gap 461. Referring to FIG. 7B, a screw 455 connected to pin 453 allows for change in gap 461 (in FIG. 7E) before the lever 452 is engaged. This allows even more adjustment in the quick clamp for engaging collets with varying outer diameters (lever handles may also be adjusted to fine tune displacements for clamping forces, screw handles large displacements based on changes in size).

Referring to FIG. 7B a luer connector 456 is operatively coupled to the clamp body 454 with a connector 464 and in one embodiment the luer connector 456 integral with a portion of the clamp body 454. In one embodiment an engagement portion 458 includes a gear 460 and a surface 462 that is received within the cassette to be supported by a bearing in the cassette.

In one embodiment the EMD 102 is removably received in the collet 112 in a radial direction and the collet 112 is removably received and positioned in the cassette. In one embodiment the EMD 102 is removably received in the collet 112 in an axial direction and the collet is removably received in the cassette. In one embodiment the EMD is removably received in the collet 112 in a radial direction and the collet 112 is non-removably positioned within cassette. In one embodiment the EMD 102 is removably received in the collet 112 in an axial direction and the collet 112 is non-removably positioned within the cassette.

Referring to FIG. 4F the drive module includes an actuator operatively coupled to a drive coupler. That is operatively coupled to a drive member in the cassette. The drive module is operatively coupled to a rail or linear support and a second actuator translates the drive module along the rail or linear support.

In one embodiment the EMD is a guidewire. In one embodiment the EMD is a catheter having a hub at a proximal end of the catheter and a flexible shaft extending from the hub toward the distal end of the catheter, wherein the shaft is more flexible than the hub. In one embodiment the catheter includes an intermediary portion between the hub and the shaft that has an intermediate flexibility that is less rigid than the hub and more rigid than the shaft.

Referring to FIGS. 8A and 8B an on-device adapter 510 holds an EMD 512 which is one embodiment is a catheter. Catheter 512 includes a hub 514 and a shaft 516. On-device adapter 510 includes a body 518 having a cavity 520 extending therein from a proximal end of body 518 that receives hub 514. Catheter hub 514 at or adjacent to a proximal end of the catheter 512 and shaft 516 extends from a region proximate hub 514 to a region proximate the distal end of the catheter 512. In one embodiment hub 514 is received within cavity 520 with a press fit or other engagement to prevent independent translation and/or rotational movement of the catheter 516 from on-device adapter 510. On-device adapter 510 includes an engagement feature 522 that engages with drive member 134 in cassette 66. In embodiment engagement feature 522 is a gear. Gear 522 is similar to gear 136 discussed herein. On-device adapter 510 and catheter 512 are translated together with cassette 66 and/or drive module 68. On-device adapter 510 and catheter 512 are rotated about a longitudinal axis of the on-device adapter 510 and catheter 512 by an actuator operatively rotating gear 134 and thereby rotating gear 522 and on-device adaptor 510 and catheter 512.

Catheter hub 514 includes a hub body 524 and in one embodiment includes a pair of wings 526 extending radially outward from hub body 524. Referring to FIGS. 8A and 8B wings 562 is received within cavity 520 of on-device adapter 510. In one embodiment catheter 512 includes a connector 528 at a proximal end thereof. In one embodiment, catheter 510 includes a strain relief section 532 intermediate hub 514 and shaft 516 that provides a transition between hub 514 and shaft 516. In one embodiment, strain relief section 532 has a proximal portion with a proximal diameter and a distal portion with a distal diameter equal or less than the proximal diameter of the shaft 516.

In one embodiment, hub 514 includes a first port to provide access to the inner lumen 534 of the catheter shaft 516 either directly or through hub shaft lumen 534. In one embodiment hub 514 includes an additional port in fluid communication with a lumen of the catheter that may for example be used for inflation of a balloon.

Shaft 516 includes a lumen 534 in fluid communication with a hub lumen 536. Connector 528 includes a lumen in fluid communication with hub lumen 536 and/or shaft lumen 534. Another EMD such as a guidewire may enter an opening in connector 528 and extend therethrough into lumen 536 of the hub and lumen 534 of the shaft. In one embodiment strain relief portion surrounds a proximal portion of shaft lumen 534. Connector 528 also allows for a fluid to be introduced therethrough into the hub lumen 536 and shaft lumen 534 to either flush out the catheter and/or provide fluid to and through the distal end of the catheter shaft 516.

To describe how catheter 512 interacts with another distal catheter, catheter 512 and its features will be referred to as the first catheter and first feature and a distal catheter and its features will be referred to as a second catheter or second feature. First shaft 516 has a given outer diameter to allow first shaft 516 to enter into a second lumen of a second catheter (not shown) and into the vasculature of a patient for diagnostic or therapeutic purposes. The outer diameter of first shaft 516 is less than the inner diameter of a second lumen of the second catheter and thereby can be inserted therein. Note that a guide catheter typically goes into an introducer sheath and not another catheter. Accordingly, a hub of a guide catheter has a geometry such that it cannot enter the introducer sheath and the patient's vasculature.

In contrast, the first hub 514 is not designed to enter into the second lumen of the second catheter or for that matter into introducer sheath lumen. In one embodiment first hub 514 has an outer periphery with a cross section at one location taken perpendicular to the longitudinal axis of the hub and/or catheter that is greater than the inner diameter of the second lumen of the a second catheter hub and/or second lumen of the second catheter. Therefore, the first hub 514 cannot enter into the second lumen of the second catheter. Further the first hub 514 geometry does not permit the proximal end of the catheter to enter into the vasculature.

Shaft 516 has a flexibility sufficient to allow the shaft 516 to bend within either a second lumen of a second catheter through which it enters and/or to allow the shaft to follow a non-straight path of the second catheter. In one embodiment the shaft 516 has flexibility sufficient to allow the shaft to bend within and follow a path of non-straight vasculature.

In one embodiment, a shaft 516 could include a stainless steel hypotube but still have sufficient flexibility to follow the non-straight path of a second catheter through which the shaft extends and/or a patient's non-straight vasculature.

In one embodiment connector 528 is a luer connector and in one embodiment the luer connector is a female luer connector. In one embodiment the luer connector has a lumen in fluid communication with the lumen of the hub to allow another EMD to pass therethrough or to allow fluid to enter the hub and catheter through the luer connector.

In one embodiment hub wings 526 are used by an operator in manual operation to hold on to hub 524. Wings 526 may be used a location device within cavity 520 of on-device adapter 510.

In one embodiment hub 514 is free of controls used to manipulate features within catheter 512 such as a wire extending to the distal end of the catheter to deflect the tip. In one embodiment catheter 512 does not include any controls used to manipulate features within the catheter such as a wire extending to the distal end of the catheter to deflect the tip.

In one embodiment on-device adapter 510 is configured to pinch an EMDs having a range of shaft outer diameters. In one embodiment a Merit Medical torque device is used as part of the on-device adapter to cover one of the following outer shaft diameter ranges: 0.009″ to 0.018″, 0.018″ to 0.038″, 0.010″ to 0.020″, 0.013″ to 0.024″, or 0.025″ to 0.040″. Where the symbol “ designates inches. Note that the torque devices provided by Merit Medical have overlapping ranges.

In one embodiment, more than one on-device adapter is used with the robotic drive system depending on the outer diameter of the shaft of the EMD to be pinched.

In one embodiment where the robotic system is controlling more than one EMD a first on-device adapter is used for a first EMD having a first outer diameter and a second on-device adapter is sued for a second EMD having a second outer diameter different than the first outer diameter of the first EMD. For example, A first on-device adapter is used to o pinch an angiographic guidewire having an outer diameter of 0.035″ or 0.038″ and the second on-device adapter is sued to pinch a microwire having an outer diameter of approx. 0.014″. An angiographic guidewire, which is used get the guide catheter in place is also called a diagnostic guidewire. And a microwire could be referred to as a micro-guidewire or simply a guidewire. For clarity the term approx. used herein is an abbreviation for the word approximately.

In one embodiment, the on-device adapter does not need to be designed to be disassembled. In one embodiment, the on-device adapter may be designed to accept a single torquer. Note that the terms torquer and torque device are used interchangeably herein and are a subset of a collet as used herein. In one embodiment, the on-device adapter provides sufficient clamping force on the torque device to withstand axial force when the on-device adapter is being advanced and retracted and withstand torsional force when the on-device is being rotated to rotate an EMD for a given procedure. The pinch or clamping force applied to the torquer by the on-device adapter is sufficient to resist slippage (axial or rotational) of the EMD being advanced and/or rotated along with the on-device adapter. In one embodiment, the on-device adapter penetrates an outer surface of the torque device body and/or deforms a surface of the torque device.

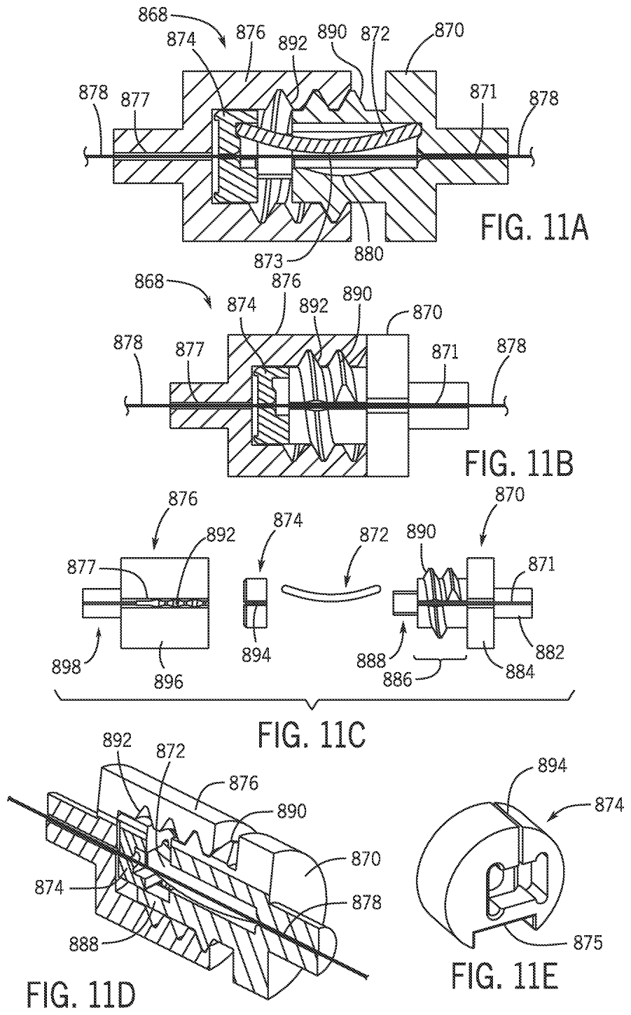

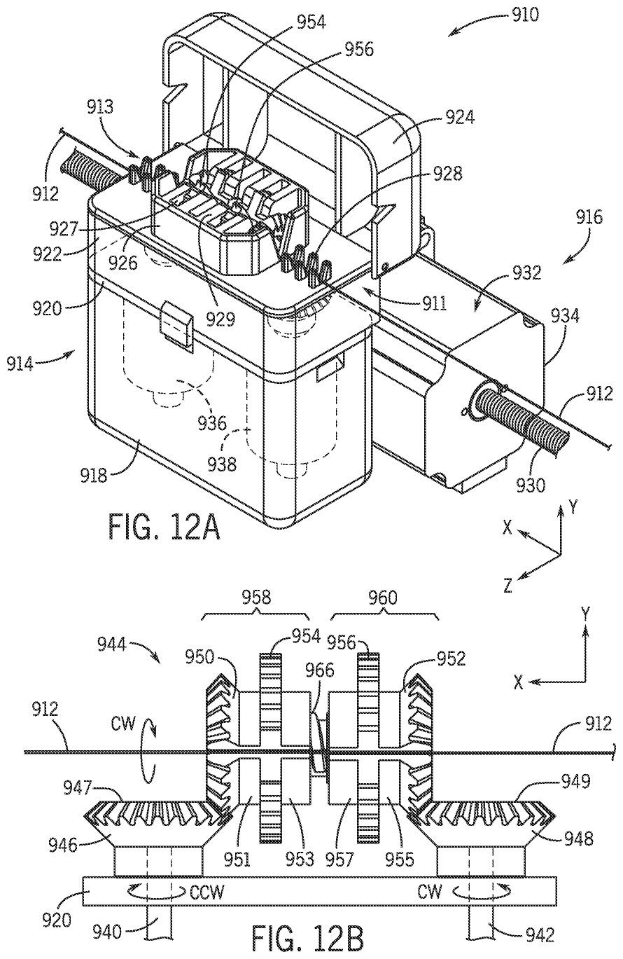

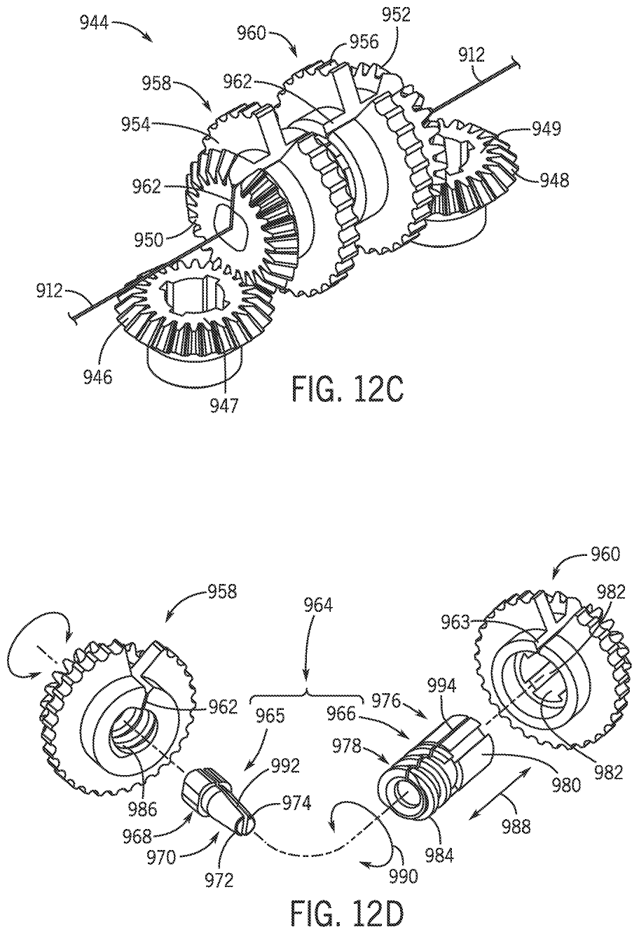

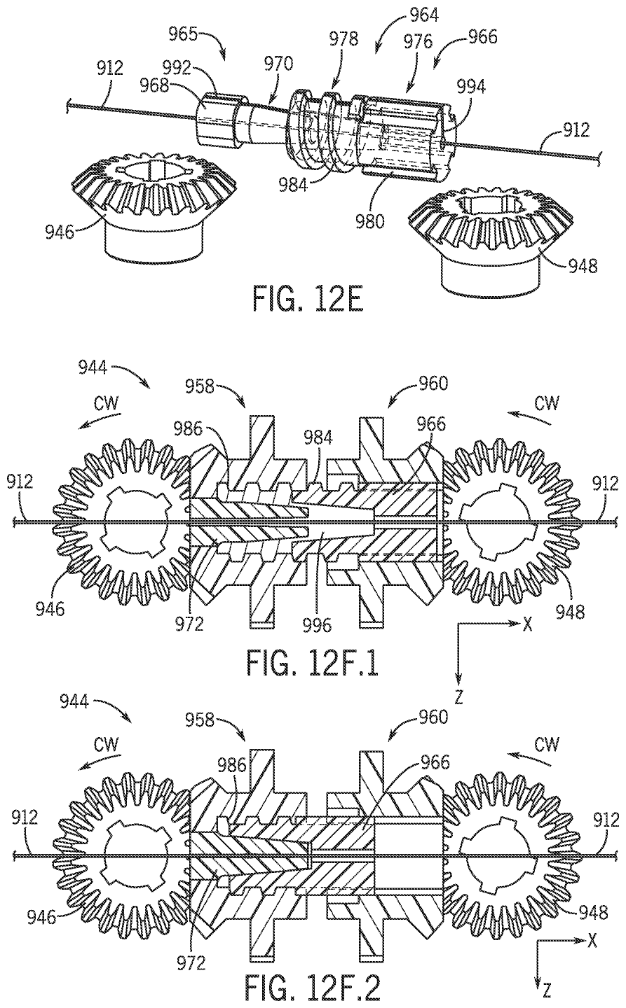

Referring to FIGS. 12A-12F.2 a robotic system 910 includes a collet 964 having a first portion 965 having a first collet coupler 958 connected thereto and a second portion 966 having a second collet coupler 960 connected thereto. Referring to FIG. 12F.1 EMD 912 is removably located within a lumen or pathway 996 defined by collet 964. A robotic drive including a drive module or base 914 having a first motor 936 and a second motor 938 operatively continuously coupled to both first collet coupler 958 and the second collet coupler 960 to operatively pinch and unpinch EMD 914 in the lumen 996 and to rotate EMD 912. As discussed herein first motor 936 and second motor 938 differentially rotate first collet coupler 958 and second collet coupler 960. Stated another way first motor 936 and second motor 938 rotate at different rates and in different directions independent of one another including where one motor rotates and the second motor does not rotate. In one embodiment both motors rotate at the same rate. In one embodiment the first motor and the second motor are continuously engaged with the first collet coupler 958 and the second collet coupler 960 respectively. In one embodiment first portion 965 and first collet coupler 958 are formed as a single component and in one embodiment they are separate components. In one embodiment second portion 966 and second collet coupler 960 are formed as a single component and in one embodiment they are separate components.

EMD robotic system 910 includes a collet employing a double-gear arrangement that releasably engages EMD 912 and rotates and translates EMD 912. In one embodiment the double-gear arrangement includes double-bevel gears. The double-gear collet-drive system 910 has a proximal end 911 and a distal end 913. As EMD 912 is moved from the proximal end 911 toward the distal end 913 the EMD 912 is being advanced into the patient and when the EMD 912 is moved from the distal end 913 toward the proximal end the EMD 912 is being retracted or withdrawn from the patient. In order to clarify directions, a rectangular coordinate system is introduced with X, Y, and Z axes. The positive Z axis is oriented in a longitudinal (axial) distal direction, that is, in the direction from the proximal end to the distal end. The X and Y axes are in a transverse plane to the Z axis, with the positive Y axis oriented up, that is, in the direction opposite of gravity, and the X axis in a direction toward the front (typically pointing toward the operator/physician who is bedside). The right-hand rule is adopted to determine the sense of rotational direction, that is, the orientation convention is determined by pointing the thumb of the right hand along the positive X, Y, and Z axis direction and then the curl of the fingers of the right hand is associated with the clockwise direction. The direction opposite the curl of the fingers of the right hand is associated with the counterclockwise direction. The terms clockwise and counterclockwise as used herein are relative terms indicating a first direction of rotation and a second direction of rotation that is opposite to the first direction of rotation. Accordingly, any use of the term clockwise and counterclockwise are to be understood to mean a first direction of rotation and a second opposing direction of rotation. The terms clockwise and counterclockwise have been used to assist in following the different rotational directions of the devices provided herein, however it is possible that the devices could be constructed with the clockwise and counterclockwise directions are reversed.

The collet-drive system 910 includes a drive module 914 that translates along an axial direction of EMD 912 and is actuated by a drive module translational drive 916. Drive module 914 includes a drive module housing 918, a mount bracket 920, a cassette 922, and a cassette cover 924. The cassette 922 includes a double-gear collet-drive housing 926 and EMD guides 928. The top of the double-gear collet-drive housing 926 includes multiple openings 927 and multiple ribs 929. The EMD guides 928 include multiple pairs of guides that act as v-shaped notches and serve as an open channel for guiding EMD 912 through the drive system. Note that the open channel is open for loading but covered when the cassette cover is in the closed position. The guides act as anti-buckling features. In one embodiment EMD guides 928 include multiple pairs of v-shaped notches or u-shaped channels that act as guides. The tops of the v-shaped or u-shaped channels may be chamfered to assist in loading the EMD 912. In one embodiment one pair of EMD guides 928 is used on the proximal side of the double-gear collet-drive housing 926 and one pair of EMD guides 928 is used on the distal side of the double-gear collet-drive housing 926. In one embodiment multiple pairs of EMD guides 928 are used on the proximal side of the double-gear collet-drive housing 926 and multiple pairs of EMD guides 928 are used on the distal side of the double-gear collet-drive housing 926.

In one embodiment robotic system 910 includes a third motor 932 (not shown) operatively coupled to collet 964 to translate collet 964 and EMD 912 along a longitudinal axis of collet 964. In one embodiment first motor 936 and second motor 938 are fixed relative to collet 964 during translation of the collet and EMD. The drive module translational drive 916 includes a lead screw 930 driven by a screw drive motor 932 (not shown) inside of a screw drive housing 934. The screw drive 930 is used to translate drive module 914 relative to fixed housing 934. In one embodiment screw drive motor 932 is a stepper motor. In one embodiment screw drive motor 932 is a servo motor. In one embodiment screw drive motor 932 is a rotational actuator powered by electrical, pneumatic, hydraulic, or other means.

In one embodiment drive module housing 918 and its contents are reusable. In one embodiment cassette 922 is consumable and meant to be disposed of after use with a single patient. In one embodiment cassette 922 may be made of a material that is sterilizable and reused.

Referring to FIGS. 12A and 12B the drive module housing 918 contains a first motor 936 that is operatively connected to and drives a first coupler 940 and a second motor 938 that is operatively connected to and drives a second coupler 942. In one embodiment first motor 936 and second motor 938 are stepper motors. In one embodiment first motor 936 and second motor 938 are servo motors. In one embodiment first motor 936 and second motor 938 are rotational actuators powered by electrical, pneumatic, hydraulic, or other means.

First coupler 940 passes through drive module housing 918 and is integrally connected to a first coupler bevel gear 946. Second coupler 942 passes through mount bracket 920 and is integrally connected to a second bevel gear 948. First motor 936, first coupler 940, and first coupler bevel gear 946 are located distally in the drive module housing 918. Second motor 938, second coupler 942, and second coupler bevel gear 948 are located proximally in the drive module housing 918. In one embodiment first coupler 940 and second coupler 942 pass through holes in mount bracket 920. In one embodiment first coupler 940 and second coupler 942 pass through rotational bearings that are mounted in mount bracket 920.

The collet-drive housing 926 contains a double-gear collet-drive assembly 944, described herein.

Referring to FIGS. 12B and 12C first driven bevel gear 950 meshes with and is driven by first coupler bevel gear 946. First driven bevel gear 950 is integrally connected to a first shaft distal portion 951, which is integrally connected to a first wheel 954, which is integrally connected a first shaft proximal portion 953, all of which form a first compound (or cluster) assembly 958. Second driven bevel gear 952 meshes with and is driven by second coupler bevel gear 948. Second driven bevel gear 952 is integrally connected to a second shaft proximal portion 955, which is integrally connected to a second wheel 954, which is integrally connected a second shaft distal portion 957, all of which form a second compound (or cluster) assembly 960.

In one embodiment a top face 947 of first coupler bevel gear 946 includes an open central hole along its central axis to receive and drive first coupler 940. Stated another way gear 946 has a hole along its longitudinal axis. In one embodiment top face 947 of first coupler bevel gear 946 is not open but sealed to prevent migration of fluids from the cassette into the base. In one embodiment a top face 949 of second coupler bevel gear 948 includes an open central hole along its central axis to receive and drive second coupler 942. In one embodiment top face 949 of second coupler bevel gear 948 is not open but sealed to prevent migration of fluids from the cassette into the base.

In one embodiment cassette 922 is removably secured to the base 914. Collet 964 is positioned within cassette 922. The first collet coupler 958 and the second collet coupler 960 are respectively coupled to the first motor 936 and the second motor 938 via a first drive coupler 940 and a second drive coupler 942 positioned within the base 914. In one embodiment first drive coupler 940 includes a shaft operatively connected to motor 936 and extending from the base in a sealed manner and is operatively connected to gear 946 that is operatively engaged with first collet coupler 958. Similarly, second drive coupler 942 includes a shaft operatively connected to motor 938 and extending from the base in a sealed manner and is operatively connected to gear 948 that is operatively engaged with second collet coupler 960.

The first compound assembly 958 contains a radial longitudinal slit 962 extending from an outer surface of the assembly and terminating at its radial center. The second compound assembly 960 contains a radial longitudinal slit 963 extending from an outer surface of the assembly and terminating at its radial center. Slits 962 and 963 allow for side or radial loading of EMD 912. In one embodiment slits 962 and 963 create radial openings with opposing nonparallel walls. In one embodiment slits 962 and 963 create approximately radial openings with opposing parallel walls. In one embodiment the outer surfaces of assemblies 958 and 960 contain v-shaped notches directed toward their center longitudinal axes that lead into the slits 962 and 963, respectively, to help guide EMD 912 for side or radial loading. It is noted that slit 962 extends through first driven bevel gear 950 and slit 963 extends through second driven bevel gear 952. First coupler bevel gear 946 meshes with and drives first driven bevel gear 950 with slit 962 without compromising performance. Second coupler bevel gear 948 meshes with and drives second driven bevel gear 952 with slit 963 without compromising performance.

Referring to FIG. 12A an outer portion of first wheel 954 and an outer portion of second wheel 956 extend through openings 927 in housing 926, making the wheels 954 and 956 accessible for manual manipulation by an operator. For example, in the event of a power loss the operator can manually rotate wheels 954 and 956 for removal of EMD 912. In one embodiment the operator can remove the collet assembly including wheels 954 and 956 from the cassette by also removing double-bevel collet-drive housing 926 from cassette allowing the operator to align the slots in the collet assembly to remove the EMD out of the cassette. In one embodiment first wheel 954 and second wheel 956 are circular disks with notches on their outer circumferential peripheries. In one embodiment first wheel 954 and second wheel 956 are circular disks with grooves on their outer circumferential peripheries. In one embodiment first wheel 954 and second wheel 956 are circular disks with knurls on their outer circumferential peripheries. In one embodiment first wheel 954 and second wheel 956 are circular disks with features that aid in manual manipulation on their outer circumferential peripheries. In one embodiment first wheel 954 and second wheel 956 are circular disks with no features, such as smooth walls, on their outer circumferential peripheries.

Referring to FIGS. 12A, 12B, and 12C the first compound assembly 958 and the second compound assembly 960 each rotate about a longitudinal axis aligned with EMD 912 and each assembly is maintained in position longitudinally by circular cutouts in ribs 929 that serve as bearings. In one embodiment open circular cutouts in ribs 929 snap over and onto both sides of first wheel 954 and second wheel 956. In other words, the first compound assembly 958 and the second compound assembly 960 can be snapped in to open cutouts in ribs 929 that partially surround the first shaft distal portion 951 and the first shaft proximal portion 953 of first compound assembly 958 and the second shaft proximal portion 955 and the second shaft distal portion 957 of second compound assembly 960. The open cutouts in ribs 929 act like thrust bearings preventing axial (longitudinal) motion and freely allowing rotational motion. The open cutouts in ribs 929 do not completely enclose the shafts 951, 953, 955, and 957. In one embodiment the open cutouts in ribs 929 offer an enclosure of 210 degrees about each of the shafts 951, 953, 955, and 957. In one embodiment the open cutouts offer an enclosure of greater than 180 degree and less than 360 degree of each of the shafts 951, 953, 955, and 957. In one embodiment the ribs with open cutouts are made of a material, such as plastic, with inherent compliance.

Referring to FIGS. 12A and 12D the double-gear collet-drive assembly 944 includes the first compound assembly 958, a collet 964 including an internal collet portion 965, an outer collet portion 966 having a screw spline, and the second compound assembly 960. Due to the snap fit feature of the open cutouts in ribs 929 the double-gear collet-drive assembly 944 (which does not include first coupler bevel gear 946 or second coupler bevel gear 948) can be manually removed from the housing 926 and reseated.

Referring to FIGS. 12D and 12E inner collet portion 965 includes a collet first section 968 integrally connected to a collet tapered second section 970 that is split into opposing cantilevered tapered jaws 972 with approximately semi-circular cross-sections. In one embodiment collet first section 968 has a prismatic shape with a generally constant radius. In one embodiment collet first section 968 has a prismatic shape with a square cross-section. In one embodiment collet 968 has a non-prismatic shape with a non-constant cross-section. Collet second section 970 extends from collet first section 968 in a frusto-conical manner such that the diameter of the second section continuously decreases from a region immediately adjacent the first section to a proximal free end 974 of the second section 970, where the proximal end 974 is furthest from the region of the second section immediately adjacent the first section 968. In one embodiment inner collet portion 965 and first compound assembly 958 are separate components. For example, collet tapered second section 970 could be a pressed metal insert into collet first section 968. In one embodiment inner collet portion 965 and first compound assembly 958 are combined into one component. Collet 964 may be any collet device known in the art including but not limited to the collet embodiments described herein.

Screw spline 966 includes a screw spline first section 976 integrally connected to a screw spline second section 978. The screw spline first section 976 contains external longitudinal spline threads 980 that mesh with the internal longitudinal spline threads 982 of the second compound assembly 960 and allow for relative translational motion in the longitudinal direction 988. The screw spline second section 978 contains external spiral circumferential screw threads 984 that mesh with internal screw threads 986 of the first compound assembly 958 and allow for relative rotational motion in the clockwise or counterclockwise directions 990. The design of the screw spline 966 with both longitudinal spline threads 980 and spiral circumferential screw threads 984 allows the screw spline 966 to be rotated and translated relative to the inner collet portion 965 while maintaining fixed longitudinal distances between first driven coupler bevel gear 950 and second driven coupler bevel gear 952 such that they can mesh, respectively with first coupler bevel gear 946 and second coupler bevel gear 948.

In one embodiment EMD 912 does not rotate while EMD 912 is being pinched and unpinched. Collet first section 968 is the section that releasably fixes EMD 912 thereto. By maintaining collet first section 968 stationary while rotating second section 966 portion EMD 912 does not rotate. Stated another way, unpinching of EMD from collet 964 without imparting any rotation to EMD 912 about the longitudinal axis of collet 964 is accomplished by maintaining internal collet portion 965 of the collet that is in direct fixed contact with EMD 192 stationary relative to the patient as outer collet portion 966 is rotated relative to inner collet portion 965 releasing EMD 192 from a fixed relationship to inner collet 965. In one embodiment it may desirable to continue to rotate EMD 912 during the beginning of the unpinch process. In this embodiment first collet section 968 rotates at a different rate than outer collet portion 966.

Referring to FIGS. 12D and 12E inner collet portion 965 contains a radial longitudinal slit 992 in collet first section 968 to allow for side or radial loading of EMD 912 into lumen 996. Longitudinal slit 992 extends radially from an outer surface of first section 968 and terminates at a radial center of inner collet portion 965. Longitudinal slit 992 extends longitudinally to second tapered section 970 through the seam of the jaws 972. Screw spline 966 contains a radial longitudinal slit 994 to allow for side or radial loading of EMD 912. Longitudinal slit 994 extends radially from an outer surface of screw spline 966 and terminates at its center.

Referring to FIG. 12F.1 in the unpinched configuration of double-gear collet-drive assembly 944 jaws 972 of collet tapered second section 972 are open and do not lock down (do not pinch) onto EMD 912. In the fully unpinched configuration screw spline 966 is in its most proximal position. In one embodiment screw spline 966 is limited to its most proximal position by a hard stop at the proximal end of its longitudinal spline. In one embodiment screw spline 966 is limited to its most proximal position by a feature, such as a flange or lip, to stop further travel in the longitudinal spline. Referring to FIG. 12F.2 in the pinched configuration of double-gear collet-drive assembly 944 jaws 972 of collet tapered second section 972 are closed together and lock (pinch) down onto EMD 912. In the fully pinched configuration screw spline 966 is in its most distal position. In one embodiment screw spline 966 is limited to its most distal position by a hard stop due to running out of thread, that is, it cannot be screwed in further as it is constrained by geometry. In one embodiment screw spline 966 is limited to its most distal position by a feature, such as a flange or lip, to stop further travel.

Referring to FIGS. 12F.1 and 12F.2 movement of inner collet portion 965 in the direction of screw spline 966 causes the jaws 972 of collet tapered second section 972 to move toward one another to pinch EMD 912. Movement of inner collet portion 965 away from the direction of screw spline 966 causes the jaws 972 of collet tapered second section 972 to move away from one another to unpinch EMD 912.

In operation double-gear collet-drive assembly 944 uses two rotational degrees of freedom from motors 936 and 938 to achieve four operations, namely, to pinch EMD 912, to unpinch EMD 912, to rotate clockwise double-gear collet-drive assembly 944, and to rotate counterclockwise double-gear collet-drive assembly 944. The four operations occur by movement of inner collet portion 965 relative to screw spline 966 based on rotation direction of first coupler 940 and rotation direction of second coupler 942.

In a first mode of operation, in which the result is the double-gear collet-drive assembly 944 rotates in a clockwise direction, first coupler 940 rotates in a counterclockwise direction and second coupler 942 rotates in a clockwise direction. In a second mode of operation, in which the result is the double-gear collet-drive assembly 944 rotates in a counterclockwise direction, first coupler 940 rotates in a clockwise direction and second coupler 942 rotates in a counterclockwise direction. In a third mode of operation, in which the result is the EMD 912 is unpinched, first coupler 940 does not rotate and second coupler 942 rotates in a counterclockwise direction. In a fourth mode of operation, in which the result is the EMD 912 is pinched, first coupler 940 does not rotate and second coupler 942 rotates in a clockwise direction. In the third mode and fourth mode of operations, the collet becomes unpinched or pinched, respectively. In one embodiment in the third mode and the fourth mode motion continues until a hard stop is reached. In one embodiment in unpinching a hard stop is reached when arriving at the end of the spline threads on the screw spline first section 976. In one embodiment in pinching a hard stop is reached when arriving at the end of the threads on the screw spline second section 978 where it meets the screw spline first section 976. For faster initiation of rotation of EMD during pinch during the fourth mode, first coupler 940 is rotated clockwise.

First motor 936 and second motor 938 can be controlled to constrain the amount of torque that each motor can apply. In one embodiment in which first motor 936 and second motor 938 are servomotors, each motor can be controlled with current limits to constrain the torque that each motor can apply. Current limits can be set at different values for the third mode and fourth mode of operations. For example, the currents can be limited to lower values for pinching than for unpinching since in unpinching static friction must be overcome.

In one embodiment double-gear collet-drive system 910 incorporates a system to prevent buckling of EMD 912 at the proximal end 911 of the collet-drive system. In one embodiment double-gear collet-drive system 910 incorporates a system to prevent buckling of EMD 912 at the distal end 913 of the collet-drive system. In one embodiment the system to prevent buckling is a tube with an inner diameter slightly larger than the outer diameter of EMD 912. In one embodiment the system to prevent buckling is a set of telescoping tubes with the inner diameter of the smallest tube slightly larger than the outer diameter of EMD 912. In one embodiment the system to prevent buckling is a side-loadable track.

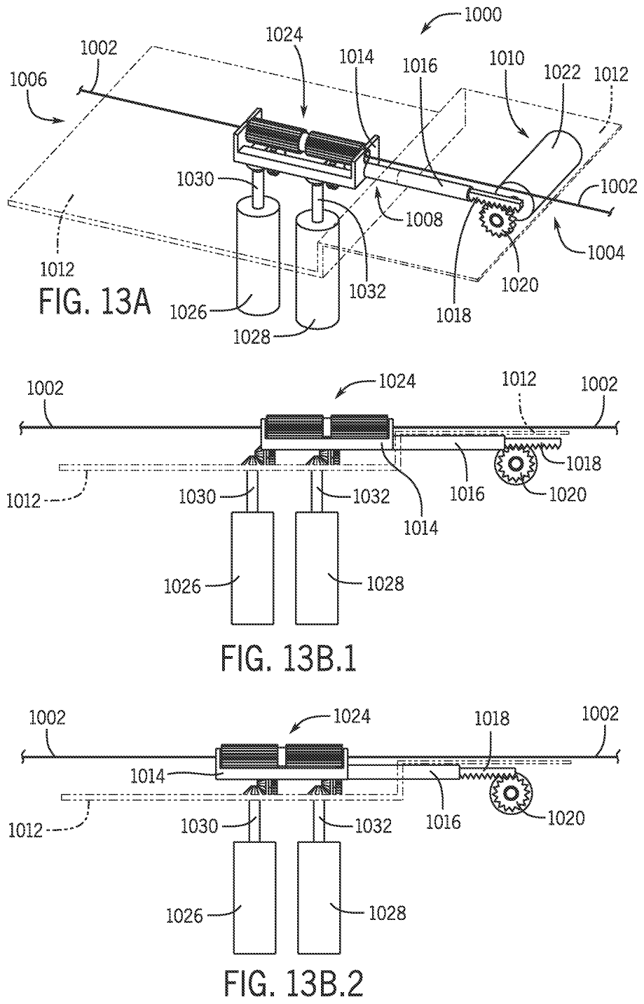

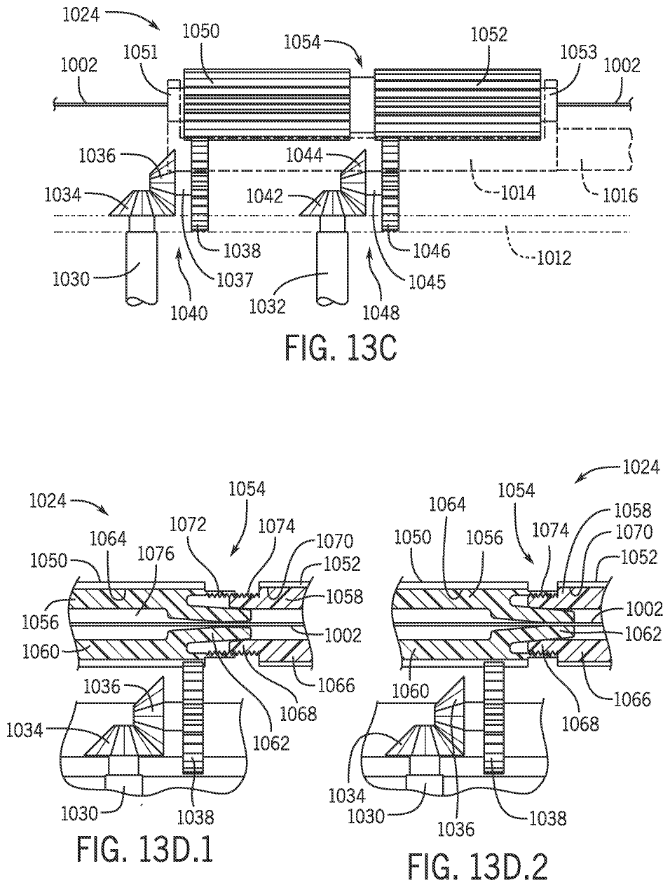

Referring to FIG. 13A a double-gear sliding collet-drive system 1000 releasably engages an elongated medical device (EMD) 1002 and rotates and translates EMD 1002. The double-gear sliding collet-drive system 1000 includes a proximal end 1004 and a distal end 1006. As EMD 1002 is moved from the proximal end 1004 toward the distal end 1006 EMD 1002 is being advanced into the patient and as EMD 1002 is moved from the distal end 1006 toward the proximal end 1004 EMD 1002 is being retracted or withdrawn from the patient.

Sliding collet-drive system 1000 includes a carrier 1008 that translates along an axial direction of EMD 1002 actuated by a carrier translational drive 1010 that is mounted to a fixed base 1012. Carrier 1008 includes a carrier housing 1014, a carrier arm 1016, and a rack 1018, all three of which are integrally connected. Carrier translational drive 1010 includes a pinion gear 1020 integrally connected to a motor shaft (not shown) of translational drive motor 1022. Translational drive motor 1022 rotates pinion gear 1020 that meshes with rack 1018 to translate carrier 1008. Linear guides or linear bearings (not shown) integrally connected to base 1012 constrain carrier 1008 to translational motion only in the proximal and distal directions along EMD 1002 axis.

Carrier housing 1014 includes a flat base plate with perpendicular side extensions on its proximal and distal ends. In one embodiment carrier housing 1014 is one integrated piece with base plate, proximal extension, and distal extension made of the same material. In one embodiment carrier housing 1014 includes a base plate, a proximal extension, and a distal extension as three separate pieces made of the same material that are integrally connected. In one embodiment carrier housing 1014 includes a base plate, a proximal extension, and a distal extension as three separate pieces made of different materials that are integrally connected. The proximal and distal extensions of carrier housing 1014 include holes that support a collet-and-rotational-drive system 1024 (described below). In one embodiment rotational bearings are mounted in the holes in the proximal and distal extensions of carrier housing 1014.

A first motor 1026 and a second motor 1028 are mounted to fixed base 1012. In one embodiment first motor 1026 and second motor 1028 are fixed relative to base 1012 during translation of collet 1056 and EMD 1002. As described herein carrier 1008 is translated with collet 1056 independently of base 1012 and first motor 1026 and second motor 1028. Stated another way, at least during one mode of operation when collet 1056 is translated along its longitudinal axis the first motor 1026 and second motor 1028 are not translated with collet 1056. First motor 1026 drives a first coupler 1030. Second motor 1028 drives a second coupler 1032. First motor 1026 and first coupler 1030 are located below or within base 1012. Second motor 1028 and second coupler 1032 are located proximally below fixed base 1012. In one embodiment first coupler 1030 and second coupler 1032 pass through holes in the fixed base 1012. In one embodiment first coupler 1030 and second coupler 1032 pass through rotational bearings and seals that are mounted in the fixed base 1012.

In one embodiment translational drive motor 1022, first motor 1026, and second motor 1028 are stepper motors however other motor types known in the art are also contemplated. In one embodiment translational drive motor 1022, first motor 1026, and second motor 1028 are servo motors. In one embodiment translational drive motor 1022, first motor 1026, and second motor 1028 are rotational actuators powered by electrical, pneumatic, hydraulic, or other means.

Referring to FIGS. 13B.1 and 13B.2 the collet-and-rotational-drive system 1024 (described below) translates relative to fixed base 1012. Referring to FIG. 13B.1 translational drive motor 1022 rotates pinion 1020 in one direction (clockwise) such that rack 1018 and hence the collet-and-rotational-drive system 1024 are translated in the proximal direction. Referring to FIG. 13B translational drive motor 1022 rotates pinion 1020 in the opposite direction (counterclockwise) such that rack 1018 and hence the collet-and-rotational-drive system 1024 are translated in the distal direction. In one embodiment collet-and-rotational-drive system 1024 translates relative to fixed base 1012 by the rack and pinion mechanism described herein. In one embodiment collet-and-rotational-drive system 1024 translates relative to fixed base 1012 by a different mechanism, such as a reciprocating mechanism in the form of a slider-crank or Scotch-yoke mechanism. An advantage of a reciprocating mechanism is that translational drive motor 1022 would not need to change direction.

Translation of collet-and-rotational-drive system 1024 is accomplished without needing to translate first motor 1026 (and first coupler 1030 and first driver bevel gear 1034) and second motor 1028 (and second coupler 1030 and second driver bevel gear 1042), both of which are mounted to fixed base 1012. Hence, inertial issues of translational acceleration and translational deceleration of first motor 1026 and second motor 1028 are avoided.

Referring to FIG. 13C first coupler 1030 is integrally connected to a first driver bevel gear 1034 that meshes with a first driven bevel gear 1036. First driven bevel gear 1036 is integrally connected to a first shaft 1037, which is integrally connected to a first spur gear 1038, all of which form a first compound (or cluster) gear assembly 1040. Second coupler 1032 is integrally connected to a second driver bevel gear 1042 that meshes with a second driven bevel gear 1044. Second driven bevel gear 1044 is integrally connected to a second shaft 1045, which is integrally connected to a second spur gear 1046, all of which form a second compound (or cluster) gear assembly 1048. First spur gear 1038 meshes with a first collet spur gear 1050 that can translate relative to first spur gear 1038. Second spur gear 1046 meshes with a second collet spur gear 1052 that can translate relative to second spur gear 1046. At the distal end of first collet spur gear 1050 is a short first shaft 1051 that is coaxially aligned and integrally connected to first collet spur gear 1050. At the proximal end of second collet spur gear 1052 is a short second shaft 1053 that is coaxially aligned and integrally connected to second collet spur gear 1052. In one embodiment first shaft 1051 is supported by a hole in the distal extension of carrier housing 1014. In one embodiment first shaft 1051 is supported by a rotational bearing mounted in a hole in the distal extension of carrier housing 1014. In one embodiment second shaft 1053 is supported by a hole in the proximal extension of carrier housing 1014. In one embodiment second shaft 1053 is supported by a rotational bearing mounted in a hole in the proximal extension of carrier housing 1014.

First collet spur gear 1050 and second collet spur gear 1052 are wide gears, that is, they are elongated gears wider than the widths of first spur gear 1038 and second spur gear 1046. In one embodiment the widths of first collet spur gear 1050 and second collet spur gear 1052 are ten times the widths of first spur gear 1038 and second spur gear 1046, respectively. In one embodiment the widths of first collet spur gear 1050 and second collet spur gear 1052 are less than ten times the widths of first spur gear 1038 and second spur gear 1046, respectively. In one embodiment the widths of first collet spur gear 1050 and second collet spur gear 1052 are greater than ten times the widths of first spur gear 1038 and second spur gear 1046, respectively.

First compound gear assembly 1040 and second compound gear assembly 1048 are supported relative to base 1012 in such a way that they are coaxially aligned and can rotate about a longitudinal axis. In one embodiment first shaft 1037 connecting first driven bevel gear 1036 and first spur gear 1038 passes through and is supported by a hole in an extension from base 1012. In one embodiment first shaft 1037 connecting first driven bevel gear 1036 and first spur gear 1038 passes through and is supported by a rotational bearing in an extension from base 1012. In one embodiment second shaft 1045 connecting second driven bevel gear 1044 and second spur gear 1046 passes through and is supported by a hole in an extension from base 1012. In one embodiment second shaft 1045 connecting second driven bevel gear 1044 and second spur gear 1046 passes through and is supported by a rotational bearing in an extension from base 1012.

Referring to FIGS. 13A and 13C the collet-and-rotational-drive 1024 includes first collet spur gear 1050 with first shaft 1051, a collet mechanism 1054 (described below), and second collet spur gear 1052 with second shaft 1053, all coaxially aligned along a longitudinal axis. In one embodiment collet-and-rotational-drive 1024 can be manually removed from carrier housing 1014 and reseated into carrier housing 1014 due to snap fit features built into the proximal side and distal side of carrier housing 1014.

In one embodiment first collet spur gear 1050 is integrally connected to a first wheel (not shown) that has a larger diameter than that of spur gear 1050 and second collet spur gear 1052 is integrally connected to a second wheel (not shown) that has a larger diameter than that of spur gear 1052. The first wheel and second wheel would be accessible for manual manipulation by an operator. For example, in the event of a power loss the operator could manually rotate the first wheel and second wheel for removal of EMD 1002. In one embodiment the first wheel and second wheel are circular disks with notches on their outer circumferential peripheries. In one embodiment the first wheel and second wheel are circular disks with grooves on their outer circumferential peripheries. In one embodiment the first wheel and second wheel are circular disks with teeth on their outer circumferential peripheries. In one embodiment the first wheel and second wheel are circular disks with features that aid in manual manipulation on their outer circumferential peripheries. In one embodiment the first wheel and second wheel are circular disks with no features, such as smooth walls, on their outer circumferential peripheries. In one embodiment first collet spur gear 1050 and the first wheel are a single integrated component made of the same material and second collet spur gear 1052 and the second wheel are a single integrated component made of the same material. In one embodiment first collet spur gear 1050 and the first wheel are separate components integrally combined and second collet spur gear 1052 and the second wheel are separate components integrally combined.

In one embodiment carrier arm 1016 can be manually removed from the proximal side of carrier housing 1014 and reconnected to the proximal side of carrier housing 1014 due to snap fit features built into the proximal side of carrier housing 1014. In one embodiment carrier arm 1016 can be manually removed from rack 1018 and reconnected to rack 1018 due to snap fit features built into the distal side of rack 1018.

In one embodiment collet-and-rotational-drive 1024 is consumable. In one embodiment collet-and-rotational-drive 1024 and carrier 1008 are consumable. In one embodiment collet-and-rotational-drive 1024 and carrier housing 1014 are consumable. In one embodiment collet-and-rotational-drive 1024, carrier housing 1014, and carrier arm 1016 are consumable.

Referring to FIG. 13D.1 and FIG. 13D.2 first collet spur gear 1050 and second collet spur gear 1052 are connected by internal components of a collet mechanism 1054. Collet mechanism 1054 includes a collet inner member 1056 and a collet outer member 1058. Collet inner member 1056 and outer member 1058 may be any collet device known in the art including but not limited to the collet embodiments described herein

Collet inner member 1056 is comprised of a first section 1060 and a second section 1062. First section 1060 of collet inner member 1056 has a cylindrical collar or sleeve shape with the center of its longitudinal axis colinear with the axis of EMD 1002 and with its outer circumferential surface integrally connected to the internal wall 1064 of first collet spur gear 1050. Second section 1062 of collet inner member 1056 has a tapered shape toward the center longitudinal axis with an internal lumen. In one embodiment second section 1062 of collet inner member 1056 includes two separated tapered jaws. In one embodiment second section 1062 of collet inner member 1056 includes more than two separated tapered jaws. In one embodiment first section 1060 and second section 1062 of collet inner member 1056 and first collet spur gear 1050 are one integrated piece. In one embodiment first section 1060 and second section 1062 of collet inner member 1056 and first collet spur gear 1050 are separate pieces that are integrally connected.

Collet outer member 1058 is comprised of a first section 1066 and a second section 1068. First section 1066 of collet outer member 1058 has a cylindrical collar or sleeve shape with the center of its longitudinal axis colinear with the axis of EMD 1002 and with its outer circumferential surface integrally connected to the internal wall 1070 of second collet spur gear 1052. Second section 1068 of collet outer member 1058 has a cylindrical collar or sleeve shape with external screw threads 1074 on its outside circumference and with the center of its longitudinal axis colinear with the axis of EMD 1002. In one embodiment first section 1066 and second section 1068 of collet outer member 1058 and second collet spur gear 1052 are one integrated piece. In one embodiment first section 1066 and second section 1068 of collet outer member 1058 and second collet spur gear 1052 are separate pieces that are integrally connected.

The external screw threads 1074 of second section 1068 of collet outer member 1058 mesh with internal screw threads 1072 of second section 1062 of collet inner member 1056. Due to meshing of internal screw threads 1072 with external screw threads 1074 rotation of collet inner member 1056 relative to collet outer member 1058 about a longitudinal axis corresponds to translation of collet inner member 1056 relative to collet outer member 1058 along a longitudinal axis. Since first collet spur gear 1050 is integrally connected to collet inner member 1056 and second collet spur gear 1052 is integrally connected to collet outer member 1058, rotation of first collet spur gear 1050 relative to second collet spur gear 1052 about a longitudinal axis corresponds to translation of first collet spur gear 1050 relative to second collet spur gear 1052 along a longitudinal axis. Rotation of first collet spur gear 1050 is accomplished by its mesh with first spur gear 1038. Rotation of second collet spur gear 1052 is accomplished by its mesh with second spur gear 1046.

To ensure continuous meshing between first collet spur gear 1050 and first spur gear 1038, first collet spur gear 1050 is made wider than first spur gear 1038. This is needed to accommodate the translation of first collet spur gear 1050 as it is rotated by first spur gear 1038 and to accommodate the translation of first collet spur gear 1050 as it is translated by carrier 1008. To ensure continuous meshing between second collet spur gear 1052 and second spur gear 1046, second collet spur gear 1052 is made wider than second spur gear 1046. This is needed to accommodate the translation of second collet spur gear 1052 as it is rotated by second spur gear 1046 and to accommodate the translation of second collet spur gear 1052 as it is translated by carrier 1008. In one embodiment first collet spur gear 1050 and second collet spur gear 1052 remain engaged with the first motor 1026 and second motor 1028 during translation of collet 1054. Stated another way first collet spur gear 1050 includes teeth having a face width of sufficient length to permit engagement of the teeth of gear 1050 with gear 1038 as gear 1050 is translated along with collet 1054 with respect to motor 1026. Similarly, second collet spur gear 1052 includes teeth having a face width of sufficient length to permit engagement of the teeth of gear 1052 with gear 1046 as gear 1052 is translated along with collet 1054 with respect to motor 1028.

Referring to FIG. 13D.1 in the unpinched configuration of the collet-and-rotational-drive system 1024 the jaws of second section 1062 of collet inner member 1056 are open and do not lock down (do not pinch) onto EMD 1002. In the fully unpinched configuration collet outer member 1058 is in its most proximal position relative to collet inner member 1056. In one embodiment collet outer member 1058 is limited to its most proximal position by a hard stop at the proximal end of its travel. In one embodiment collet outer member 1058 is limited to its most proximal position by a feature, such as a flange or lip, to stop further travel in the longitudinal direction. Referring to FIG. 13D.2 in the pinched configuration of the collet-and-rotational-drive system 1024 the jaws of second section 1062 of collet inner member 1056 are closed together and lock (pinch) down onto EMD 1002. In the fully pinched configuration collet outer member 1058 is in its most distal position relative to collet inner member 1056. In one embodiment the collet outer member 1058 is limited to its most distal position by a hard stop due to running out of thread, that is, it cannot be screwed in further as it is constrained by geometry. In one embodiment the collet outer member 1058 is limited to its most distal position by a feature, such as a flange or lip, to stop further longitudinal travel.

The principle of operation of the collet-and-rotational-drive system 1024 is similar to that of the collet of the double-gear collet-drive assembly 944 of FIG. 12C and FIG. 12 D. As first collet spur gear 1050 and second collet spur gear 1052 are rotated such that they are threaded toward one another, the inner surface of second section 1068 of collet outer member 1058 presses against second section 1062 of collet inner member 1056 and pinches down on EMD 1002. As first collet spur gear 1050 and second collet spur gear 1052 are rotated such that they are unthreaded away from one another, the inner surface of second section 1068 of collet outer member 1058 relaxes and stops pressing against second section 1062 of collet inner member 1056 and unpinches EMD 1002.

In operation the double-gear collet-and-rotational drive system 1024 uses two rotational degrees of freedom from motors 1026 and 1028 to achieve four operations, namely, to pinch EMD 1002, to unpinch EMD 1002, to rotate clockwise double-gear collet-and-rotational drive system 1024, and to rotate counterclockwise double-gear collet-and-rotational drive system 1024. The four operations occur by movement of collet inner member 1056 relative to collet outer member 1058 based on rotation direction of first coupler 1030 and rotation direction of second coupler 1032.

In a first mode of operation, in which the result is the double-gear collet-and-rotational drive system 1024 rotates in a clockwise direction, first coupler 1030 rotates in a clockwise direction and second coupler 1032 rotates in a counterclockwise direction. In a second mode of operation, in which the result is the double-gear collet-and-rotational drive system 1024 rotates in a counterclockwise direction, first coupler 1030 rotates in a counterclockwise direction and second coupler 1032 rotates in a clockwise direction. In a third mode of operation, in which the result is the EMD 1002 is unpinched, first coupler 1030 rotates in a clockwise direction and second coupler 1032 rotates in a clockwise direction. In a fourth mode of operation, in which the result is the EMD 1002 is pinched, first coupler 1030 rotates in a counterclockwise direction and second coupler 1032 rotates in a counterclockwise direction. In the third mode and fourth mode of operations, collet inner member 1056 unpinches or pinches, respectively, EMD 1002 until a hard stop is reached.

In one embodiment pinching and unpinching of collet mechanism 1054 is synchronized with the rotational position of the shaft of translational drive motor 1022.

In one embodiment, components of the double-gear sliding collet-drive system 1000 contain longitudinal slits (not shown) to enable radial or side loading of EMD 1002 into collet lumen 1076.

Robotic system 1000 in one embodiment includes a pinch/unpinch mode, a rotation mode and a translation mode. The pinch/unpinch mode, rotation mode and translation mode may occur individually or simultaneously. In one embodiment rotation mode and the translation mode occur simultaneously.

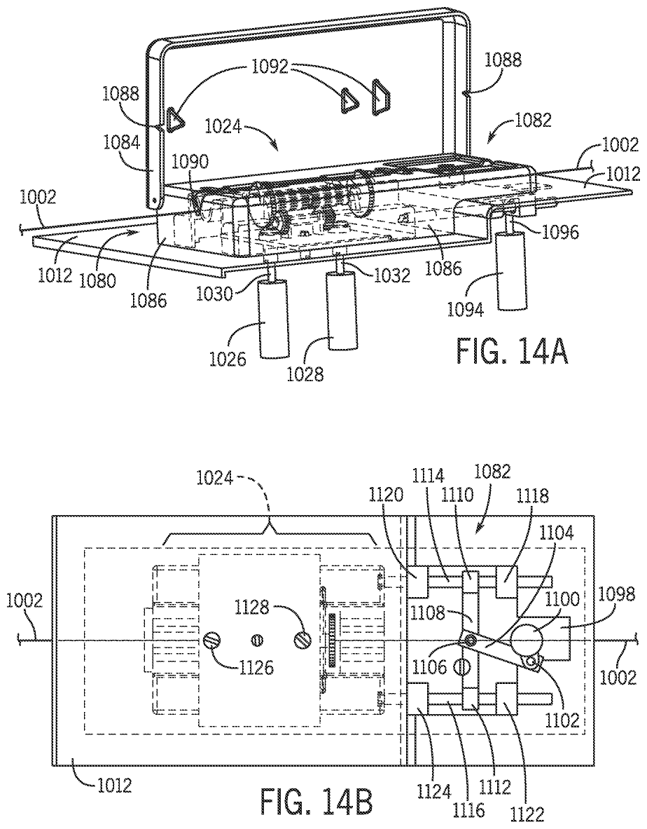

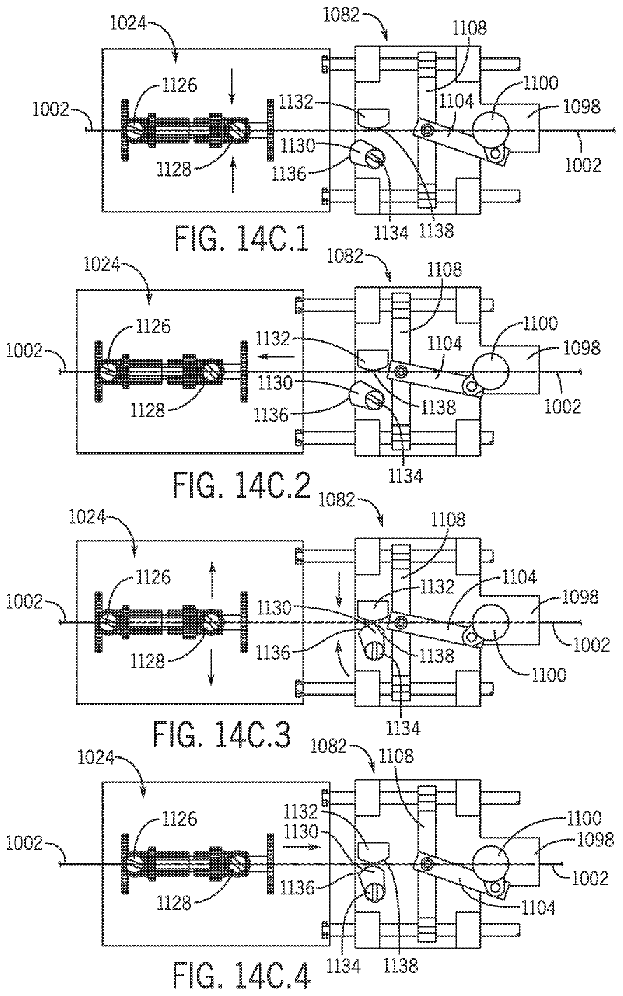

Referring to FIG. 14A one embodiment of a double-gear sliding collet-drive system with a reset mechanism is indicated. A disposable cassette 1080 is releasably mounted to a fixed base 1012 and includes the collet-and-rotational-drive system 1024 (described above) located distally and a reset mechanism 1082 located proximally. Reset mechanism 1082 (described below) is designed to advance, retract, and hold an EMD 1002. Cassette 1080 includes a top cassette cover 1084 and a bottom cassette housing 1086. In one embodiment cassette cover 1084 is connected to cassette housing 1086 by hinges at the back that allow the cover to rotate open and rotate close from the front. In one embodiment cassette cover 1084 is connected to cassette housing 1086 by hinges at the front that allow the cover to rotate open and rotate close from the back. In one embodiment cassette cover 1084 is connected to cassette housing 1086 by hinges that allow the cover to rotate open and close from the side. In one embodiment cassette cover 1084 is connected to cassette housing 1086 by fasteners that allow the cover to be opened and closed by rotation, by translation, or by a combination of rotation and translation relative to the housing 1086. In one embodiment cassette cover 1084 is connected to cassette housing 1086 by press-fit features that allow the cover to be opened and closed by rotation, by translation, or by a combination of rotation and translation relative to the housing 1086. In one embodiment cassette cover 1084 is connected to cassette housing 1086 by press-fit features that allow the cover to be removed from the housing 1086 and reseated to the housing 1086.

The proximal and distal sides of the cassette cover 1084 include cover notches 1088 that allow for free passage of EMD 1002. The proximal and distal sides of the cassette housing 1086 include housing notches 1090 that match the positions of cover notches 1088. In one embodiment cover notches 1088 and housing notches 1090 are triangular-shaped cutouts that allow for free passage of EMD 1002. In one embodiment cover notches 1088 and housing notches 1090 are arbitrarily shaped cutouts that allow for free passage of EMD 1002. The underside of cassette cover 1084 includes cover ribs 1092. When cassette cover 1084 is closed cover ribs 1092 seat EMD 1002 into alignment notches 1090 in cassette housing 1086 and maintain EMD 1002 vertical position in said alignment grooves or channels that maintain EMD 1002 lateral position.

As described above the collet-and-rotational-drive system 1024 is actuated by a first motor 1026 driving a first coupler 1030 and a second motor 1028 driving a second coupler 1032. The reset mechanism 1082 is actuated by a reset mechanism motor 1094 that drives a reset mechanism coupler 1096. In one embodiment reset mechanism motor 1094 is a stepper motor. In one embodiment reset mechanism motor 1094 is a servo motor. In one embodiment reset mechanism motor 1094 is a rotational actuator powered by electrical, pneumatic, hydraulic, or other means.

Referring to FIG. 14B the underside of fixed base 1012 is indicated. The reset mechanism 1082 is built into a reset mechanism frame 1098 that is integrally connected to fixed base 1012. Reset mechanism coupler 1096 is integrally connected to a reset mechanism crank 1100 that can rotate relative to frame 1098 and base 1012. In one embodiment reset mechanism coupler 1096 passes through a hole in reset mechanism frame 1098. In one embodiment reset mechanism coupler 1096 passes through a rotational bearing that is mounted in reset mechanism frame 1098. Reset mechanism crank 1100 is connected by a first revolute joint 1102 to a connecting link 1104. Connecting link 1104 is connected by a second revolute joint 1106 to a cross-slider 1108. Cross-slider 1108 is constrained to longitudinal translational motion (that is, translational motion only along the axis of EMD 1002) by a cross-slider first linear bearing 1110 and a cross-slider second linear bearing 1112, both of which are integrally connected to cross-slider 1108. First linear bearing 1110 is a prismatic joint that can translate relative to a first guide 1114 and second linear bearing 1112 is a prismatic joint that can translate relative to a second guide 1116. The distal ends of first guide 1114 and second guide 1116 are integrally connected to fixed base 1012 and as such guides 1114 and 1116 are fixed.

A proximal first linear bearing 1118 and a distal first linear bearing 1120 are integrally mounted to the front corners of reset mechanism frame 1098. A proximal second linear bearing 1122 and a distal second linear bearing 1124 are integrally mounted to the rear corners of reset mechanism frame 1098. First guide 1114 can translate relative to proximal first linear bearing 1118 and distal first linear bearing 1120. Second guide 1116 can translate relative to proximal second linear bearing 1122 and distal second linear bearing 1124. Since the four bearings 1118, 1120, 1122, and 1124 are integrally mounted to reset mechanism frame 1098, reset mechanism 1082 can translate longitudinally relative to fixed base 1012.

In one embodiment first coupler 1030 has a first coupler slotted end 1126 that seats into a slotted receiver of a shaft integrally connected to first driver bevel gear 1034 and second coupler 1032 has a second coupler slotted end 1128 that seats into a slotted receiver of a shaft integrally connected to second driver bevel gear 1042. (See FIG. 13C)

Referring to FIGS. 14C.1, 14C.2, 14C.3, and 14C.4 a sequence of steps indicates the operation of linear position mechanism 1082, which includes a reset clamping cam 1130 that can rotate and a clamp support 1132 that is fixed. Reset cam 1130 rotates about a vertical axis by a reset cam coupler 1134. In one embodiment reset cam coupler 1134 about which reset cam 1130 rotates is driven by a motor (not shown). In one embodiment reset cam coupler 1134 about which reset cam 1130 rotates is driven by a mechanism actuated by reset mechanism motor 1094. In one embodiment reset cam coupler 1134 has a slotted end that seats in a receiver in cam 1130. Reset cam 1130 has a curved outer surface 1136. In one embodiment curved outer surface 1136 of reset cam 1130 has a convex geometry. In one embodiment curved outer surface 1136 of reset cam 1130 has a circular arc geometry. Holding cam 1132 has a curved outer surface 1138. In one embodiment curved outer surface 1138 of holding cam 1132 has a convex geometry. In one embodiment curved outer surface 1138 of holding cam 1132 has a circular arc geometry.

In operation reset cam 1130 can be in a closed position or an open position. In the closed position reset cam 1130 is in an opposing position relative to holding cam 1132. In one embodiment in the closed position there is no gap between reset cam outer surface 1136 and holding cam outer surface 1138 and the two surfaces 1136 and 1138 are in contact. In one embodiment in the closed position there is a gap between reset cam outer surface 1136 and holding cam outer surface 1138 with the gap distance less than the diameter of EMD 1002. In the closed position EMD 1002 is pinched between reset cam outer surface 1136 and holding cam outer surface 1138, such that EMD 1002 is prevented from translating longitudinally. In one embodiment reset cam outer surface 1136 and holding cam outer surface 1138 include an elastomeric or other deformable or compliant material that deforms about the EMD in the closed position. In the open position reset cam 1130 is rotated away from holding cam 1132 such that there is a gap between reset cam outer surface 1136 and holding cam outer surface 1138. In the open position reset cam 1130 does not contact EMD 1002, such that EMD 1002 is unconstrained to translate longitudinally at the location of holding cam 1132. In one embodiment reset cam 1130 rotates 60 degrees away from holding cam 1132 in the open position. In one embodiment reset cam 1130 rotates less than 60 degrees away from holding cam 1132 in the open position. In one embodiment reset cam 1130 rotates more than 60 degrees away from holding cam 1132 in the open position.

Referring to FIG. 14C.1 the collet-and-rotational-drive system 1024 is pinching down on EMD 1002, reset cam 1130 is in the open position, and cross-slider 1108 is in a proximal position relative to reset mechanism frame 1098. As a result of this step, EMD 1002 is pinched in collet-and-rotational-drive system 1024.

Referring to FIG. 14C.2 the collet-and-rotational-drive system 1024 is pinched on EMD 1002, reset cam 1130 is in the open position, and cross-slider 1108 is translating distally from a proximal position relative to the reset mechanism frame 1098. In one embodiment cross-slider 1108 is translating distally due to clockwise rotation of reset mechanism crank 1100 by reset mechanism motor 1094. As a result of this step, collet-and-rotational-drive system 1024 advances distally, meaning EMD 1002 advances distally.

Referring to FIG. 14C.3 the collet-and-rotational-drive system 1024 is unpinching EMD 1002, reset cam 1130 is in the closed position, and cross-slider 1108 is in its most distal position relative to reset mechanism frame 1098. As a result of this step, EMD 1002 is unpinched in collet-and-rotational-drive system 1024.

Referring to FIG. 14C.4 the collet-and-rotational-drive system 1024 is unpinched from EMD 1002, reset cam 1130 is in the closed position, and cross-slider 1108 is translating proximally relative to reset mechanism frame 1098. In one embodiment cross-slider 1108 is translating proximally due to counterclockwise rotation of reset mechanism crank 1100 by reset mechanism motor 1094. As a result of this step, collet-and-rotational-drive system 1024 advances proximally and the system resets and can subsequently start over (to FIG. 14C.1).

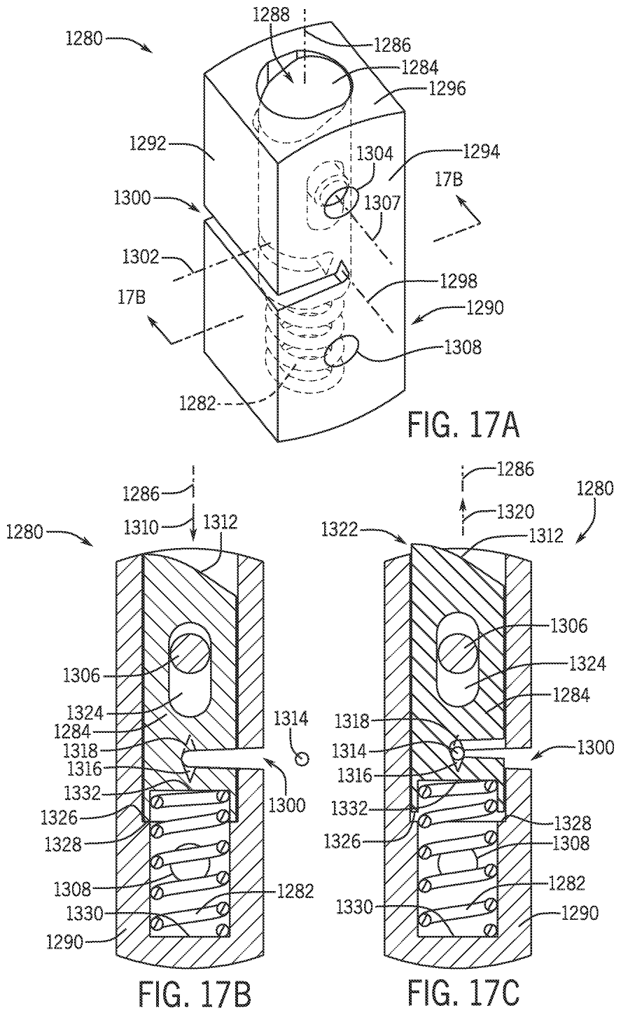

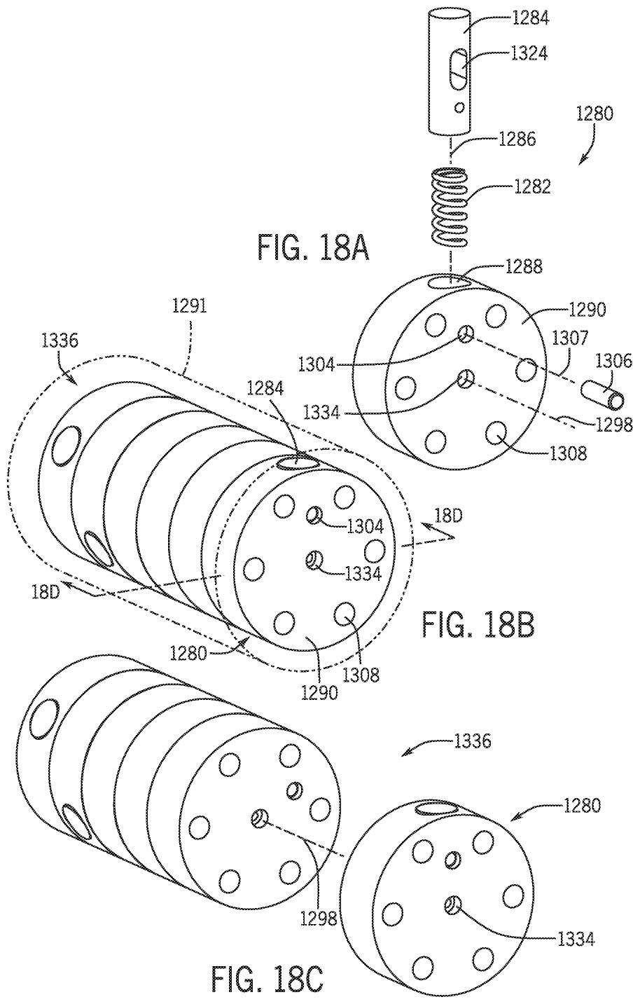

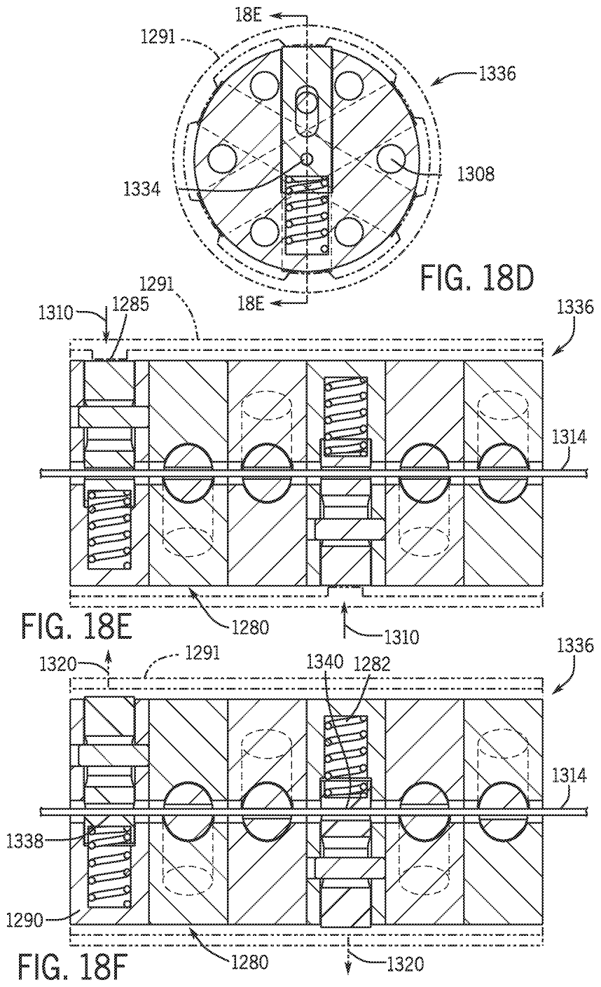

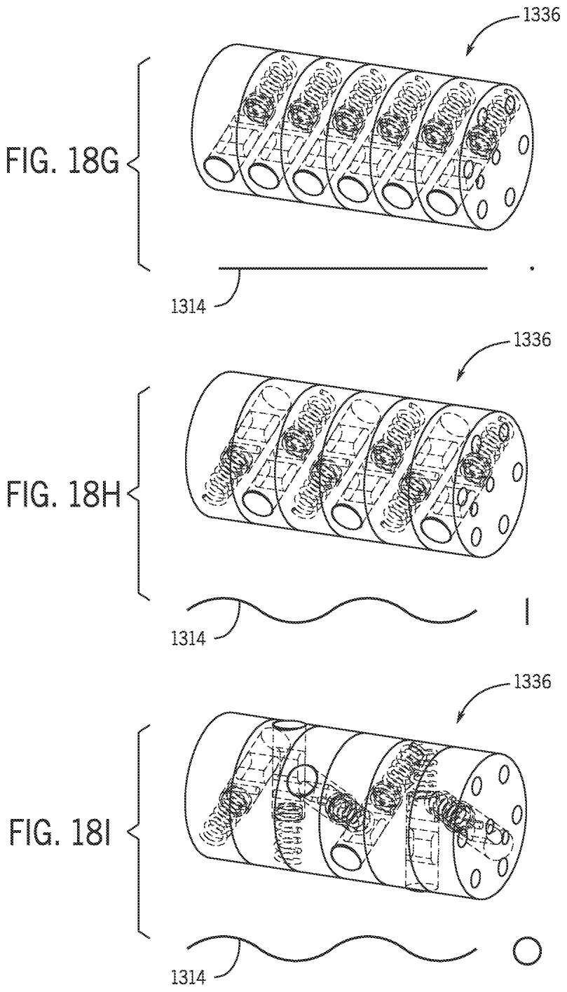

Referring to FIG. 17A a single plunger collet system 1280 that can releasably engage an EMD includes a spring 1282 and a plunger 1284 that is movably positioned along a plunger axis 1286 within a receiving cavity 1288 of a housing 1290. In the embodiment of FIG. 17A housing 1290 is a rectangular prism with a first lateral face 1292, a second lateral face 1294, and a convex top face 1296. First lateral face 1292 is parallel to the plane defined by the plunger axis 1286 and an EMD axis 1298. Second lateral face 1294 is parallel to the plane defined by the plunger axis 1286 and a perpendicular axis 1302, where the perpendicular axis 1302 is perpendicular to the plunger axis 1286 and EMD axis 1298. In one embodiment housing 1290 is a rectangular prism with the top face 1296 and opposite bottom face being rectangular planes. In one embodiment, the embodiment of FIG. 18A, housing 1290 is a cylindrical disk with plunger axis 1286 aligned with a diametric axis of the disk, with the embodiment of FIG. 17A being a section removed from such a cylindrical disk. Referring to FIGS. 18B and 18D an outer housing 1291 is located about housings 1290. Outer housing 1291 includes a plurality of cammed surfaces on an inner wall that operatively engage respective plungers 1284 as outer housing 1291 is rotated about its longitudinal axis relative to housings 1290. In one embodiment the longitudinal axes of housings 1290 are co-linear with the longitudinal axis of outer housing 1291. In one embodiment at least a portion of outer housing 1291 and/or a portion of housings 1290 is arcuate and/or circular.

First lateral face 1292 of housing 1290 has a slit 1300 oriented in the plane defined by EMD axis 1298 and perpendicular axis 1302 extending from face 1292 and terminating at EMD axis 1298 through housing 1290 from second lateral face 1294 to its opposite face. In one embodiment the walls of slit 1300 are parallel. In one embodiment the walls of slit 1300 are nonparallel, such as v-shaped walls with a vertex toward EMD axis 1298. In one embodiment slit 1300 has a lead-in chamfer at first lateral face 1292. In one embodiment slit 1300 has no lead-in chamfer at first lateral face 1292.

Second lateral face 1294 of housing 1290 includes a plunger pin hole 1304 for a plunger pin 1306 (not shown in FIG. 17A) and a guide hole 1308 for an alignment pin (not shown). Plunger pin hole 1304 is aligned with a plunger pin axis 1307 parallel to EMD axis 1298 in the plane defined by the plunger axis 1286 and EMD axis 1298 extending through housing 1290 from second lateral face 1294 and terminating at the opposite outside face. Guide hole 1308 is aligned with an axis parallel to EMD axis 1298 in the plane defined by plunger axis 1286 and EMD axis 1298 extending through the housing wall from second lateral face 1294 and terminating at the opposite wall interior face of cavity 1288 in housing 1290. In one embodiment guide hole 1308 is a well or cap hole in second lateral face 1294 and does not terminate at the opposite wall interior face of cavity 1288 in housing 1290. In the embodiment of single plunger collet system 1280 in FIG. 17A guide hole 1308 is not needed. Guide hole 1308 is used for alignment of multi-plunger assemblies.

Referring to FIG. 17B plunger collet system 1280 is indicated in an unpinched configuration in which EMD 1314 is not operatively fixed to collet 1280. An applied force 1310 acts on a top surface 1312 of plunger 1284 pushing plunger 1284 down in cavity 1288 of housing 1290, compressing spring 1282 located below plunger 1284 with its long axis oriented along plunger axis 1286. With plunger 1284 depressed fully into cavity 1288, in one embodiment a bottom outer surface 1326 of plunger 1284 touches a lip 1328 in cavity 1288 of housing 1290, thereby limiting further movement of plunger 1284. With contact between surface 1326 and lip 1328 the plunger 1284 reaches its most depressed configuration in which spring 1282 is in its maximum compression state. In this case a plunger notch 1316 in plunger 1284 is furthest apart from a housing notch 1318 in housing 1290 and EMD 1314 can be moved into the open slit 1300 in the direction of plunger axis 1286. In one embodiment plunger notch 1316 is a v-shaped channel or groove with its vertex pointed down. In one embodiment plunger notch 1316 is a well with its concavity pointed down. In one embodiment plunger notch 1316 is a generally downward depression with arbitrary geometry. In one embodiment housing notch 1318 is a v-shaped channel or groove with its vertex pointed up. In one embodiment housing notch 1318 is a well with its concavity pointed up. In one embodiment housing notch 1318 is a generally upward depression with arbitrary geometry.

With EMD 1314 fully inserted into the well of slit 1300 at plunger axis 1286, applied force 1310 is removed. Referring to FIG. 17C plunger collet system 1280 is indicated in a pinched configuration in which EMD 1314 is not free to move relative to the collet, trapped between plunger notch 1316 and housing notch 1318 at the well of slit 1300 at plunger axis 1286 due to a restoring force 1320 from spring 1282 that pushes up on plunger 1284. In the pinched configuration there is a gap between bottom outer surface 1326 of plunger 1284 and lip 1328 in cavity 1288 of housing 1290. In addition, in the pinched configuration a portion 1322 of plunger 1284 protrudes outside of top face 1296 of housing 1290 and is exposed.

Referring to FIG. 17B and FIG. 17C plunger collet system 1280 is a normally closed collet, meaning without application of an applied force 1310 the collet is in a pinched configuration.

The bottom of compression spring 1282 is in contact with a bottom inner surface 1330 of cavity 1288 of housing 1290. The top of compression spring 1282 is in contact with a bottom inner surface 1332 of plunger 1284. In one embodiment at the bottom inner surface 1332 of plunger 1284 there is a pocket or cup that receives the top of spring 1282 and constrains the top of spring 1282 by lip 1328. The outer diameter of spring 1282 is smaller than the inner diameter of cavity 1288 at the bottom of housing 1290 to allow freedom for compression. In one embodiment the outer diameter of spring 1282 is smaller than the inner diameter of cavity 1288 at the bottom of housing 1290 and larger than the diameter corresponding to buckling or bending of the spring to prevent buckling or bending of the spring. In one embodiment one compression spring 1282 is utilized. In one embodiment multiple springs, such as two nested springs, are used.

Plunger 1284 includes a plunger slot 1324 oriented along plunger axis 1286 allowing plunger 1284 to translate along plunger axis 1286 relative to housing 1290 constrained by plunger pin 1306 and the walls of the cavity 1288 in housing 1290. To unpinch collet 1280 plunger 1284 is depressed down by application of applied force 1310 to the top surface 1312 of plunger. In operation plunger 1284 is a cam follower with its top surface 1312 being the follower surface in contact with a cam (not shown) pushing down on the cam follower with applied force 1310. An outer member (not shown) with an internal cam is in contact with the top surface 1312 of plunger 1284. By rotation of the outer member relative to housing 1290 the internal cam of the outer member pushes down on the top surface 1312 thereby depressing plunger 1284 and unpinching EMD 1314 in collet 1280.

Referring to FIG. 18A a single plunger collet system 1280 operates with the same principle with housing 1290 being a circular disk with a center hole 1334 for EMD 1314 (not shown). The embodiment of FIG. 18A includes six guide holes 1308 arranged symmetrically about EMD axis 1298 at the same radial distance away from EMD axis 1298.

Referring to FIG. 18B a multi-plunger collet system 1336 is indicated in an assembled configuration of six single plunger assemblies 1280, each being the embodiment of FIG. 18A, cascaded in series with each one progressively rotated relative to another about EMD axis 1298. In one embodiment each of the six single plunger assemblies 1280 in the series is progressively rotated (that is, sequentially rotated in the same direction) by 60 degrees from one another such that guide holes 1308 are aligned. In this embodiment each single plunger assembly is rotated 60 degrees from assembly before it in the series. That is, if the first assembly is considered the reference at 0 degrees, the second assembly is rotated 60 degrees clockwise relative to the first assembly, the third assembly is rotated 120 degrees clockwise relative to the first assembly, the fourth assembly is rotated 180 degrees clockwise relative to the first assembly, the fifth assembly is rotated 240 degrees clockwise relative to the first assembly, and the sixth assembly is rotated 300 degrees clockwise relative to the first assembly. Thus, the plungers of the first and fourth assemblies are in opposite directions (180 degrees apart), the plungers of the second and fifth assemblies are in opposite directions (180 degrees apart), and the plungers of the third and sixth assemblies are in opposite directions (180 degrees apart).

Referring to FIG. 18C a multi-plunger collet system 1336 is indicated in the assembled configuration shown in 18B with the first single plunger assembly 1280 separated off. Again, system 1336 includes six single plunger assemblies (1280), each being the embodiment of FIG. 18A, cascaded in series with each one progressively rotated by 60 degrees about EMD axis 1298 relative to the assembly before it.

Referring to FIG. 18D the end view of the assembled multi-plunger system 1336 of FIG. 18B is indicated with solid lines for the first single plunger assembly 1280 and phantom lines for the second through sixth single plunger assemblies 1280 with each single plunger assembly progressively rotated by 60 degrees about EMD axis 1298 relative to the assembly before it such that the guide holes 1308 align. The three visible single plunger assemblies correspond to the first and fourth assemblies, the second and fifth assemblies, and the third and sixth assemblies, with each pair being in opposite directions (180 degrees apart). The center hole 1334 of the six single plunger assemblies 1280 align for axial loading of EMD 1314. In one embodiment six single plunger assemblies 1280 are used each progressively rotated by 60 degrees about EMD axis 1298 relative to the assembly before it. In one embodiment four single plunger assemblies 1280 are used each progressively rotated by 90 degrees about EMD axis 1298 relative to the assembly before it. In one embodiment three single plunger assemblies 1280 are used each progressively rotated by 120 degrees about EMD axis 1298 relative to the assembly before it. In one embodiment two single plunger assemblies 1280 are used with the second assembly rotated by 180 degrees about EMD axis 1298 relative to the first assembly. In one embodiment two single plunger assemblies 1280 are used with the second assembly rotated by less than 180 degrees about EMD axis 1298 relative to the first assembly. In one embodiment two single plunger assemblies 1280 are used with the second assembly rotated by more than 180 degrees about EMD axis 1298 relative to the first assembly. In one embodiment more than two single plunger assemblies 1280 are used each progressively rotated by an arbitrary number of degrees about EMD axis 1298 relative to the assembly before it. In an example of this embodiment using four single plunger assemblies 1280 if the first assembly is considered the reference at 0 degrees, the second assembly is rotated 45 degrees clockwise relative to the first assembly, the third assembly is rotated 135 degrees clockwise relative to the first assembly, and the fourth assembly is rotated 180 degrees clockwise relative to the first assembly. This embodiment allows radial loading of an EMD within the collet. In one embodiment the single plunger assemblies 1280 of the multi-plunger collet system are identical. In one embodiment the single plunger assemblies 1280 of the multi-plunger collet system are not identical.

Referring to FIG. 18E the unpinched configuration of a multi-plunger collet system 1336 with six single plunger assemblies 1280 requires an external applied force 1310 applied to each plunger 1284 from an outer member cam (not shown). In the unpinched configuration there is no contact of EMD 1314 between the plunger and housing at any single plunger assembly 1280 in the multi-plunger system 1336.

Referring to FIG. 18F the pinched configuration of a multi-plunger collet system 1336 with six single plunger assemblies 1280 is indicated. In the pinched configuration there is contact of EMD 1314 between the plunger and housing at each single plunger assembly 1280 in the multi-plunger system 1336 due to reaction force 1320 from each compression spring 1282. Being that each single plunger assembly 1280 is sequentially rotated relative to the assembly before it, contact on EMD 1314 occurs at different surfaces giving more torque capability of the collet system 1336. In the embodiment of FIG. 18F contact occurs at a portion of the bottom surface 1338 of EMD 1314 in the first single plunger assembly 1280 (shown at left) and contact occurs at a portion of the top surface 1340 of EMD 1314 in the fourth (from left) single plunger assembly 1280. Contact at different surface portions of EMD 1314 occurs at each single plunger assembly 1280 meaning there is contact at different portions longitudinally along the EMD.

Referring to FIGS. 18G, 18H, and 18I a multi-plunger collet system 1336 in the pinched configuration with six single plunger assemblies 1280 is indicated with EMD 1314 in a side view and a front view. Referring to FIG. 18G a multi-plunger collet system 1336 with six single plunger assemblies 1280 all oriented in the same direction is indicated. The side view of EMD 1314 is a straight line and the front view of EMD 1314 is a single point. Referring to FIG. 18H a multi-plunger collet system 1336 with six single plunger assemblies 1280 each oriented 180 degrees apart from the assembly before it is indicated. The side view of EMD 1314 is an approximately sinusoidal line in a plane and the front view of EMD 1314 is a single point moving up and down along a vertical line. Referring to FIG. 18I a multi-plunger collet system 1336 with six single plunger assemblies 1280 each progressively oriented 60 degrees apart from the assembly before it is indicated. The side view of EMD 1314 is an approximately sinusoidal line in a plane and the front view of EMD 1314 is a single point moving along the circumference of a circle.

Compared to the torque carrying ability of the multi-plunger collet system 1336 of FIG. 18G when pinched, the torque carrying ability of the multi-plunger collet system 1336 of FIG. 18H when pinched is increased. Due to the 180 degree offsets of the single plunger assemblies 1280 in the multi-plunger collet system of FIG. 18H the EMD 1314 adopts a tortuous configuration that goes up and down in a side view with the top and bottom of the vertical line in a front view having the most resistive torque (with the neutral device axis being in the center of the line). Compared to the torque carrying ability of the multi-plunger collet system 1336 of FIG. 18H when pinched, the torque carrying ability of the multi-plunger collet system 1336 of FIG. 18I when pinched is further increased. Due to the 60 degree offsets of the single plunger assemblies 1280 in the multi-plunger collet system of FIG. 18H the EMD 1314 adopts a configuration that has a spiral path, that is, helix shape, with the EMD always away from the central axis 1298 of the EMD giving the most resistive torque.

The deformation of the EMD 1314 in the pinched configuration of the multi-plunger collet system 1336 is a function of the through hole diameter in the center of the plunger housing, the gap (clearance) between the plunger and plunger housing, and the force applied by the spring mechanism.

In one embodiment a series of pinching elements in a collet for robotic actuation where the pinching elements are independently actuated. The actuation mechanism such as a cam is such that instead of actuating all of the elements together, their actuation is not all together such as sequentially actuated. This feature acts to lower actuation force.

In one embodiment multi-plunger collet system 1336 consisting of multiple pinching elements are rotationally clocked to each other in order to increase the overall torque holding capability of the collet. Rotationally clocked refers to placing the pinching elements at various angles in a plane perpendicular to the longitudinal axis of the collet 1336.

Referring to FIG. 18B collet 1336 includes an inner member that defines a pathway receiving an EMD 1314 and an outer member a plurality of engagement members 1284 releasably engaging EMD 1314 as the inner member is moved relative to the outer member. In one embodiment a spring 1282 biases engagement member 1284. In one embodiment spring 1282 biases engagement member 1284 away from the pathway in one embodiment spring 1282 biases engagement member 1284 toward the pathway. In one embodiment engagement members 1284 are normally closed or located within the pathway and require to be moved to an open position to insert an EMD. In one embodiment engagement members 1284 are normally open or located outside of the pathway and require to be moved to a closed position to engage the EMD. In one embodiment engagement members 1284 sequentially engage the EMD. Referring to FIG. 18I in one embodiment engagement members 1284 are offset circumferentially about the EMD. Referring to FIG. 18G in one embodiment engagement members 1284 are offset axially. Referring to FIG. 18H in one embodiment a first engagement member is positioned 180 degrees from a second engagement member. In one embodiment engagement members 1284 are independent and not directly connected to one another. In one embodiment movement of the inner member relative to the outer member is rotational. In one embodiment movement of the inner member relative to the outer member is translational. In one embodiment the movement of the inner member and outer member relative to one another is robotic. In one embodiment movement of the inner member and outer member relative to one another is manual. Referring to FIGS. 18H and 18I in one embodiment engagement members 1284 are offset radially about the EMD forming a tortuous path. Referring to FIG. 18H in one embodiment the tortuous path is in a single plane. Referring to FIG. 18I in one embodiment the tortuous path is not in a single plane.

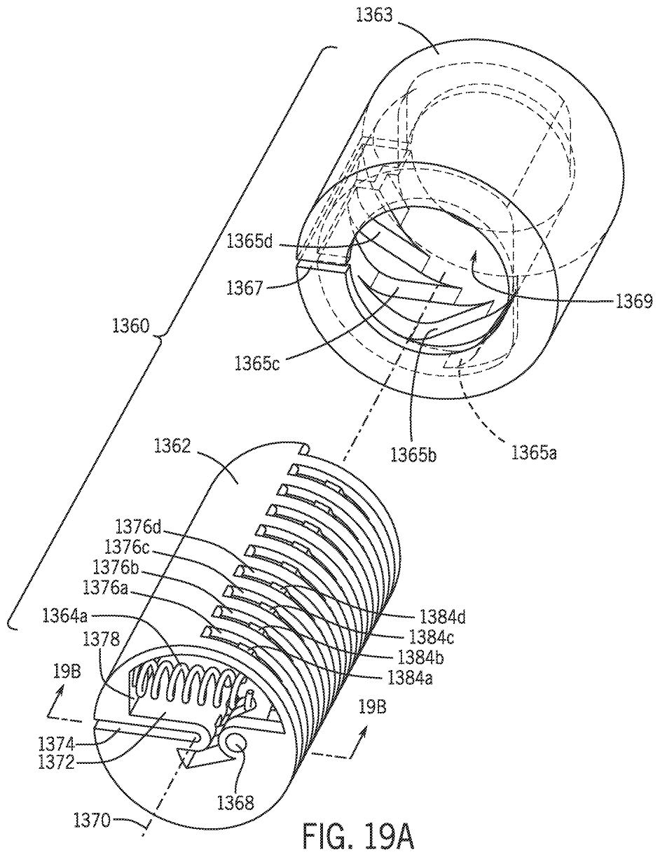

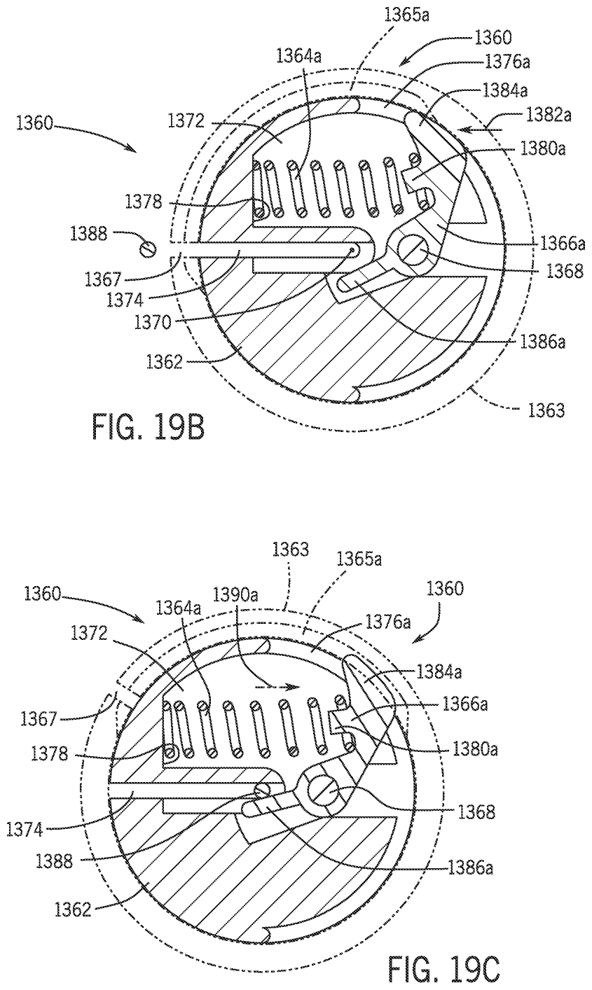

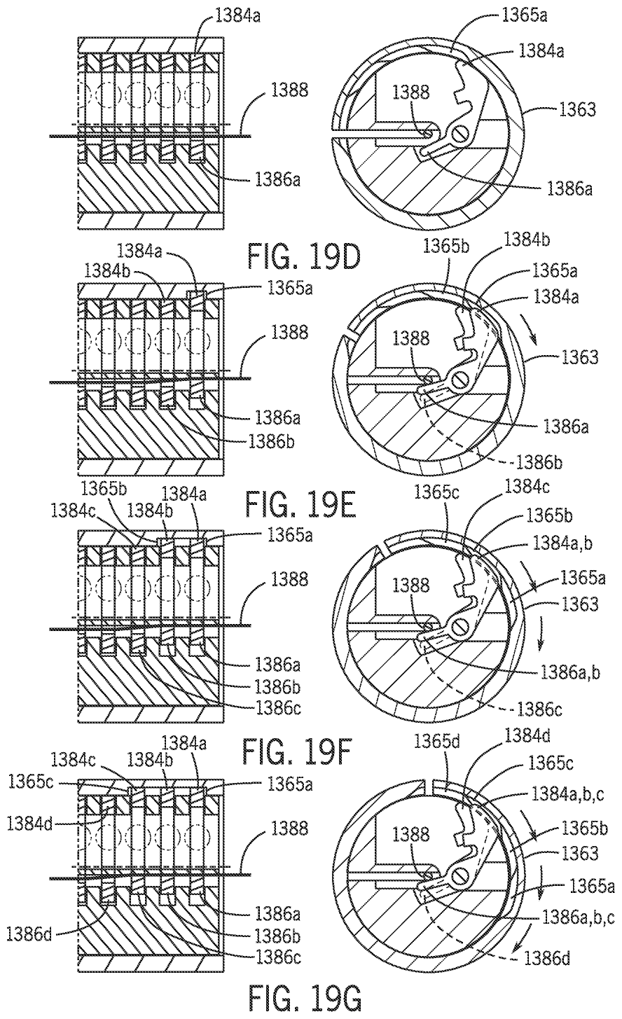

Referring to FIGS. 19A, 19B, 19C, and 19E an opposing pad collet system 1360 that can releasably engage an EMD 1388 includes an inner housing 1362, an outer housing 1363, a plurality of springs 1364a,b,c, . . . , a plurality of levers 1366a,b,c, . . . , and a pivot pin 1368. In one embodiment inner housing 1362 of collet system 1360 is in the shape of a right circular cylinder with its longitudinal axis oriented along the EMD axis 1370. Inner housing 1362 includes an internal cavity 1372, a radial longitudinal slit 1374, and a plurality of circumferential slits 1376a,b,c, . . . . In one embodiment outer housing 1363 is in the shape of a right circular cylinder with its longitudinal axis oriented along the EMD axis 1370. Outer housing 1363 includes a radial longitudinal slit 1367, an internal cavity 1369, and a plurality of cam surfaces 1365a,b,c, . . . on the inner surface (interior wall) of outer housing 1363. In one embodiment the outer housing 1363 is a cylindrical tube with a wall thickness greater than 10 percent of the inner diameter with a plurality of cam surfaces 1365a,b,c, . . . on the inner surface. In one embodiment the outer housing 1363 is a cylindrical tube with a wall thickness less than 10 percent of the inner diameter with a plurality of cam surfaces 1365a,b,c, . . . on the inner surface. (Referring to FIGS. 19A-19G the wall thicknesses of outer housing 1363 are representative. Note that the geometry of outer housing 1363 in FIG. 19A differs from the representative cross-section of FIG. 19B-19G.) The outer diameter of inner housing 1362 is smaller than the diameter of the internal cavity 1369 of outer housing 1363 such that in the assembled embodiment inner housing 1362 is located interior to outer housing 1363.

In one embodiment the longitudinal axis of inner housing 1362 is co-linear with the longitudinal axis of outer housing 1363. In one embodiment at least a portion of outer housing 1363 and/or a portion of inner housing 1362 is arcuate and/or circular. In one embodiment all levers 1366a,b,c, . . . rotate about a single pivot pin 1368. In one embodiment multiple pivot pins 1368a,b,c, . . . are used, where lever 1366a rotates about pin 1368a, lever 1366b rotates about pin 1368b, etc. In one embodiment the plurality of cam surfaces 1365a,b,c, . . . are incrementally spaced along a longitudinal axis around the inner circumference of outer housing 1363. In one embodiment the plurality of cam surfaces 1365a,b,c, . . . are grooves or recesses incrementally spaced along a longitudinal axis around the inner circumference of outer housing 1363.

Circumferential slits 1376a,b,c, . . . of inner housing 1362 are oriented parallel to a plane perpendicular to EMD axis 1370. In the embodiment of FIG. 19A nine circumferential slits 1376a,b,c, . . . , i are indicated in which nine arms 1384a,b,c, . . . i of levers 1366a,b,c, . . . i are correspondingly exposed. In other embodiments a different number of circumferential slits is used with a corresponding number of arms exposed. For example, in one embodiment one circumferential slit 1376a is used in which arm 1384a of lever 1366a is exposed. In one embodiment two circumferential slits 1376a,b are used in which arms 1384a,b of levers 1366a,b are correspondingly exposed. In one embodiment more than one circumferential slit 1376 is used. In one embodiment circumferential slits 1376a,b,c, . . . extend radially inward from an outer surface of inner housing 1362 through to internal cavity 1372 of inner housing 1362. In one embodiment circumferential slits 1376a,b,c, . . . extend radially inward from an outer surface of inner housing 1362 through to the interior of inner housing 1362 that is not part of cavity 1372. In one embodiment circumferential slits 1376a,b,c, . . . extend radially inward from an outer surface of inner housing 1362 through to the internal cavity 1372 of inner housing 1362 and through to the interior of inner housing 1362 that is not part of cavity 1372. In one embodiment the walls of slits 1376a,b,c, . . . are parallel. In one embodiment the walls of circumferential slits 1376a,b,c, . . . are nonparallel. In one embodiment circumferential slits 1376a,b,c, . . . have lead-in chamfers at the outer surface of inner housing 1362. In one embodiment circumferential slits 1376a,b,c, . . . have no lead-in chamfers at the outer surface of inner housing 1362.

Radial longitudinal slit 1367 of outer housing 1363 extends from an outer surface of outer housing 1363 and terminates at inner surface of internal cavity 1369 of outer housing 1363. The gap between the walls of radial longitudinal slit 1367 is larger than the diameter of an EMD 1388 allowing an EMD 1388 to enter. In one embodiment the walls of radial longitudinal slit 1367 are parallel. In one embodiment the walls of radial longitudinal slit 1367 are nonparallel, such as v-shaped walls with a vertex toward EMD axis 1370. In one embodiment radial longitudinal slit 1367 has a lead-in chamfer at the outer surface of outer housing 1363. In one embodiment radial longitudinal slit 1367 has no lead-in chamfer at the outer surface of outer housing 1363.

Radial longitudinal slit 1374 of inner housing 1362 extends from an outer surface of inner housing 1362 and terminates at its radial center corresponding to EMD axis 1370 and extends longitudinally through inner housing 1362. The gap distance between the walls of radial longitudinal slit 1374 is larger than the diameter of an EMD 1388 allowing an EMD 1388 to enter. In one embodiment the walls of radial longitudinal slit 1374 are parallel. In one embodiment the walls of radial longitudinal slit 1374 slits are nonparallel, such as v-shaped walls with a vertex toward EMD axis 1370. In one embodiment radial longitudinal slit 1374 has a lead-in chamfer at the outer surface of inner housing 1362. In one embodiment radial longitudinal slit 1374 has no lead-in chamfer at the outer surface of inner housing 1362.

Springs 1364a,b,c, . . . are compression springs, such as coil springs, located in the internal cavity 1372 of inner housing 1362. One end of springs 1364a,b,c, . . . is constrained by an internal wall 1378 of cavity 1372 of inner housing 1362. The other end of springs 1364a,b,c, . . . is seated over and extends into protrusions 1380a,b,c, . . . of levers 1366a,b,c, . . . . In one embodiment protrusions 1380a,b,c, . . . of levers 1366a,b,c, . . . extend into one end coil of springs 1364a,b,c, . . . . In one embodiment protrusions 1380a,b,c, . . . of levers 1366a,b,c, . . . extend into more than one end coil of springs 1364a,b,c, . . . . In one embodiment protrusions 1380a,b,c, . . . of levers 1366a,b,c, . . . are operatively connected to one end coil of springs 1364a,b,c, . . . . In one embodiment protrusions 1380a,b,c, . . . of levers 1366a,b,c, . . . are operatively connected to more than one end coil of springs 1364a,b,c, . . . . In one embodiment one compression spring 1364 is used. In one embodiment multiple compression springs are used. In one embodiment, the number of springs equals the number of levers. In one embodiment a collar or sleeve surrounding each spring 1364a,b,c, . . . is used to prevent buckling or bending of the springs.

In the assembled configuration springs 1364a,b,c, . . . are in compression. In operation, cam surfaces 1365a,b,c, . . . on the inner surface (interior wall) of outer housing 1363 operatively engage respective arms 1384a,b,c, . . . of levers 1366a,b,c, . . . that are exposed in slits 1376a,b,c, . . . as outer housing 1363 is rotated about its longitudinal axis relative to inner housing 1362. Referring to FIG. 19B opposing pad collet system 1360 is indicated in an unpinched configuration in which EMD 1388 is not operatively fixed to collet 1360. In this configuration radial longitudinal slit 1367 of outer housing 1363 is aligned with radial longitudinal slit 1374 of inner housing. An applied force 1382a acts on arm 1384a of lever 1366a such that lever 1366a is rotated counterclockwise about pivot pin 1368 with spring 1364a in cavity 1372 of inner housing 1362 under compression. Due to the position of lever 1366a, pad 1386a of lever 1366a is oriented away from EMD axis 1370 and from radial longitudinal slit 1374 near EMD axis 1370. In this unpinched configuration EMD 1388 can be moved into the radial longitudinal slit 1367 and into radial longitudinal slit 1374 in the direction of EMD axis 1370. In one embodiment outer housing 1363 is rotated relative to inner housing 1362 by an actuator (not shown). The actuator rotating outer housing 1363 relative to inner housing 1362 in one embodiment is in the drive module and in one embodiment is in the cassette.

To pinch and unpinch opposing pad collet system 1360 lever 1366a pivots about pivot pin 1368 through a limited range of motion. In one embodiment the angular range of motion of lever 1366a is less than 10 degrees. In one embodiment the angular range of motion is greater than 10 degrees. Lever 1366a acts as a first-class lever with its pivot between an effort and a load. An effort or input force 1382a is applied to an arm 1384a of lever 1366a. A load or output force acts at pad 1386a of lever 1366a.

With EMD 1388 fully inserted into radial longitudinal slit 1374, applied force 1382a is removed. Referring to FIG. 19C opposing pad collet system 1360 is indicated in a pinched configuration in which EMD 1388 is not free to move relative to the collet, trapped between pad 1386a and a wall of radial longitudinal slit 1374 due to a restoring force 1390a from spring 1364a that pushes up on arm 1384a of lever 1366a. In one embodiment in the pinched configuration the outside end of arm 1384a protrudes into the circumferential slit 1376a of inner housing 1362 and is exposed.

Referring to FIG. 19B and FIG. 19C opposing pad collet system 1360 is a normally closed collet, meaning without application of an applied force 1382a the collet is in a pinched configuration.

In operation arm 1384a of lever 1366a is a cam follower with an outer surface of arm 1384a being the follower surface in contact with a cam (inner surface of outer housing 1363) pushing on the cam follower with applied force 1382a. Outer member 1363 with an internal cam is in contact with the outer surface of arm 1384a. By rotation of the outer housing 1363 relative to inner housing 1362 the internal cam of the outer member pushes on the outer surface of arm 1384a, exposed in circumferential slit 1376a, thereby rotating lever 1366a and moving pad 1386a of lever 1366a away from EMD axis 1370 and unpinching EMD 1388 in collet 1360. In one embodiment with a single circumferential slit 1376a the cam includes a finger or tab that presses against the outer surface of arm 1384a. In one embodiment with multiple circumferential slits 1376a,b,c, . . . the cam includes multiple fingers or tabs that press against outer surfaces of multiple arms 1384a,b,c, . . . . In one embodiment multiple levers 1366a,b,c, . . . are used with their pads 1386a,b,c, . . . pinching the EMD 1388 at multiple locations longitudinally. In one embodiment contact of the EMD 1388 occurs between the pad 1386a of a single lever 1366a along the length of the collet system.

Referring to FIGS. 19D-19G the sequence of incremental pinching of opposing pad collet system 1360 is indicated. (In the figures on the right springs 1364a,b,c, . . . are present but not shown; in the figures on the left springs 1364a,b,c, . . . are not numbers but indicated by lightly dashed circles.) Referring to FIG. 19D, the opposing pad collet system 1360 is indicated in an unpinched configuration for radial loading of EMD 1388. There is no contact of pads 1386a,b,c, . . . with EMD 1388 since inner wall of outer housing 1363 maintains arms 1384a,b,c, . . . of levers 1366a,b,c, . . . in a configuration that compresses springs 1364a,b,c, . . . in the maximum compressive state during operation. Referring to FIG. 19E, a first increment of rotation (corresponding to one clockwise arrow) of outer housing 1363 relative to inner housing 1362 corresponds to engagement of pad 1386a of lever 1366a with EMD 1388 as a result of rotation of lever 1366a due to the recess of cam 1365a on the inner surface of outer housing 1363. Spring 1364a is slightly relaxed from its maximum compressive state and is the source of the force between pad 1386a and EMD 1388. In this first increment of rotation all other pads 1386b,c, . . . of levers 1366b,c, . . . remain in the unpinched configuration. In this first increment of rotation it is not possible for EMD 1388 to be removed from opposing pad collet system 1360 since radial longitudinal slit 1367 of outer housing 1363 is not aligned with radial longitudinal slit 1374 of inner housing 1362. Referring to FIG. 19F, a second increment of rotation (corresponding to two clockwise arrows) of outer housing 1363 relative to inner housing 1362 corresponds to engagement of pads 1386a and 1386b with EMD 1388 as a result of rotation of levers 1366a and 1366b due to the recesses of cams 1365a and 1365b on the inner surface of outer housing 1363. Springs 1364a and 1364b are slightly relaxed from their maximum compressive state and are the source of the force between pads 1386a and 1386b and EMD 1388. In this second increment of rotation all other pads 1386c,d, . . . of levers 1366c,d, . . . remain in the unpinched configuration. Referring to FIG. 19G, a third increment of rotation (corresponding to three clockwise arrows) of outer housing 1363 relative to inner housing 1362 corresponds to engagement of pads 1386a,b,c with EMD 1388 as a result of rotation of levers 1366a,b,c due to the recesses of cams 1365a,b,c on the inner surface of outer housing 1363. Springs 1364a,b,c are slightly relaxed from their maximum compressive state and are the source of the force between pads 1386a,b,c and EMD 1388. In this third increment of rotation all other pads 1386d,e, . . . of levers 1366d,e, . . . remain in the unpinched configuration. (Note: In FIGS. 19E-19G the EMD 1388 is illustrated with an exaggerated bias in the places where there is engagement.)

In one embodiment rotation of 20 degrees of outer housing 1363 relative to inner housing 1362 corresponds to an increment of rotation for engagement of a pad 1386a,b,c, . . . of corresponding lever 1366a,b,c, . . . with EMD 1388. In one embodiment rotation of less than 20 degrees of outer housing 1363 relative to inner housing 1362 corresponds to an increment of rotation for engagement of a pad 1386a,b,c, . . . of corresponding lever 1366a,b,c, . . . with EMD 1388. In one embodiment rotation of more than 20 degrees of outer housing 1363 relative to inner housing 1362 corresponds to an increment of rotation for engagement of a pad 1386a,b,c, . . . of corresponding lever 1366a,b,c, . . . with EMD 1388.

Referring to FIG. 20A, a collet-drive system 1500 that can rotate, translate, and pinch an EMD 1502 includes a collet 1504, a collet engagement member 1506, a first drive module 1508, and a second drive module 1510. Collet-drive system 1500 may also be referred to as a quick release collet with two linear drives and axial spline engagement.

Collet 1504 has a collet first member 1512 that has a first engagement portion 1514. Collet 1504 has a collet second member 1516 that is driven.

Collet engagement member 1506 has a second engagement portion 1518.

Collet first member 1512 and collet engagement member 1506 move between an engaged position and a disengaged position. Referring to FIG. 20C collet first member 1512 and collet engagement member 1506 are indicated in a disengaged position.

First engagement portion 1514 engages second engagement portion 1518 as collet first member 1512 and collet engagement member 1506 are moved to the engaged position. Referring to FIGS. 20C-20G collet first member 1512 and collet engagement member 1506 are indicated in an engaged position.

Rotation of collet first member 1512 with respect to collet second member 1516 in a first direction 1520 in the engaged position pinches an EMD 1502 within the collet 1504 and rotation of collet first member 1512 with respect to collet second member 1516 in a second direction 1522 opposite the first direction 1520 unpinches the EMD 1502 within the collet 1504.

In collet-drive system 1500 the first engagement portion 1514 includes a plurality of splines that extend circumferentially about at least a portion of the collet first member 1512. The second engagement portion 1518 includes a plurality of members operatively engaging the plurality of splines of the first engagement portion 1514.

In one embodiment collet second member 1516 is connected to a bevel gear 1524 that meshes with and is driven by a capstan bevel gear 1526. In one embodiment collet second member 1516 is driven by a coupler.

In one embodiment the plurality of splines of first engagement portion 1514 includes external spline teeth that extend longitudinally. In one embodiment the plurality of members of second engagement portion 1518 includes internal spline teeth that extend longitudinally and mesh with the external spline teeth that extend longitudinally of the plurality of splines of first engagement portion 1514.

Collet engagement member 1506 is integrally connected to first drive module 1508 and oriented such that its centerline is aligned longitudinally with the axis of EMD 1502.

First drive module 1508 and second drive module 1510 translate longitudinally relative to a fixed lead screw 1528 (illustrated as reference 76 in FIG. 3) and are driven independently by a first actuator 1530 and a second actuator 1532 (identified as translation motors 64 in FIG. 3), respectively. In one embodiment lead screw 1528 is a ball screw. In one embodiment, first drive module 1508 and second drive module 1510 are driven independently by belt drives. In one embodiment first actuator 1530 is a motor powered by electrical, pneumatic, hydraulic, or other means. In one embodiment second actuator 1532 is a motor powered by electrical, pneumatic, hydraulic, or other means.

Referring to FIG. 20A collet-drive system 1500 is connected to the overall robotic system 24. In particular, the connections of lead screw 1528, first actuator 1530, second actuator 1532, first drive module 1508, and second drive module 1510 to the overall robotic system is illustrated.

In one embodiment translation of first drive module 1508 is accomplished as follows. A drive shaft of first actuator 1530 is integrally connected to a first actuation pulley 1534 that drives a first belt 1536 that drives a first nut pulley 1538 that is integrally connected to a first nut-bearing assembly 1540 that meshes with lead screw 1528 and is integrally connected to first drive module 1508. Similarly, in one embodiment translation of second drive module 1510 is accomplished as follows. A drive shaft of second actuator 1532 is integrally connected to a second actuation pulley 1544 that drives a second belt 1546 that drives a second nut pulley 1548 that is integrally connected to a second nut-bearing assembly 1550 that meshes with lead screw 1528 and is integrally connected to second drive module 1510.

First drive module 1508 includes a clamp and rotational drive mechanism that acts both to clamp/unclamp an EMD as well as to translate the EMD along its longitudinal axis. In one embodiment the clamp and rotational drive mechanism includes drive tire 1558 and an idler tire 1568. In one embodiment drive tire 1558 is driven as follows. A driver gear 1552 meshes with a drive tire gear 1554 that is integrally connected to a drive tire capstan 1556 that is integrally connected to drive tire 1558. It is contemplated that other clamp and translational devices known in the art may be employed as well.

Referring to FIGS. 20A and 20B in one embodiment driver gear 1552 is driven by a third actuator 1560 that is incorporated internal to first drive module 1508. In one embodiment third actuator 1560 is a motor powered by electrical, pneumatic, hydraulic, or other means.

In one embodiment rotation of driver gear 1552 is accomplished as follows. A drive shaft of third actuator 1560 is integrally connected to a third actuation pulley 1562 (supported by a bearing) that drives a second belt 1564 that drives a driver gear pulley 1566 (supported by a bearing) that is integrally connected to driver gear 1552.

First drive module 1508 includes a straddle rocker 1570 and a spring 1572. Straddle rocker 1570 rotates about a pivot 1574 that is parallel to the axis of drive tire 1558 and idler tire 1568. Spring 1572 is a tension spring with one end connected to a rocker distal post 1575 integrally connected to straddle rocker 1570 and one end connected to a driver gear extension post 1576 that extends from driver gear 1552. Straddle rocker 1570 is a spring-loaded bell crank, that is, a spring-loaded lever with two arms and pivot 1574. One arm of straddle rocker 1570 is integrally connected to rocker distal post 1575 at its free end. One arm of straddle rocker 1570 supports idler tire 1568 at its free end.

Second drive module 1510 includes driven capstan bevel gear 1526 and capstan 1527. Capstan bevel gear 1526 is integrally connected to capstan 1527 that is driven by an actuator (not shown). Second drive module 1510 is integrally connected to an extension link 1578 that extends out from the far end (that is, end farthest from lead screw 1528) of second drive module 1510 in a direction toward first drive module 1508 and parallel to lead screw 1528 and to EMD 1502. In one embodiment extension link 1578 is a rectangular bar with its length greater than its width and its width greater than its height (thickness). Extension link 1578 includes a first lip 1580 and a second lip 1581. In one embodiment first lip 1580 and second lip 1581 are rectangular bar projections, like flanges, oriented up and perpendicular to extension link 1578. In one embodiment first lip 1580 is located at the proximal end of extension link 1578 and second lip 1581 is located near the proximal end of extension link 1578 such that there is a gap between the inside faces of first lip 1580 and second lip 1581.

In one embodiment collet-drive system 1500 includes a cassette (not shown) that includes collet 1504, collet engagement member 1506, drive tire 1558, and idler tire 1568.

Operation of collet-drive system 1500 consists of multiple states, as described herein.

Referring to FIG. 20C collet-drive system 1500 is indicated in a driving state (first state). In the driving state collet 1504 pinches EMD 1502, collet 1504 rotates EMD 1502, first drive module 1508 and second drive module 1510 move together maintaining the same separation distance, the spline teeth of first engagement portion 1514 and second engagement portion 1518 do not mesh (that is, are not engaged), and drive tire 1558 and idler tire 1568 are separated and do not grip EMD 1502. In the driving state rocker distal post 1575 is in contact with the inside face of first lip 1580 and straddle rocker 1570 is positioned to keep idler tire 1568 separated from drive tire 1558.

Referring to FIG. 20D collet-drive system 1500 is indicated in a collet lock state (second state). In the collet lock state collet 1504 pinches EMD 1502, first drive module 1508 and second drive module 1510 move toward one another reducing their separation distance (for example, second drive module 1510 moves toward a fixed first drive module 1508), the spline teeth of first engagement portion 1514 mesh with spline teeth of second engagement portion 1518 (that is, they are engaged, although not fully), and drive tire 1558 and idler tire 1568 are slightly separated from one another and do not grip EMD 1502. In the collet lock state rocker distal post 1575 is in contact with the inside face of first lip 1580 and straddle rocker 1570 rotates moving idler tire 1568 toward drive tire 1558 but the tires do not grip EMD 1502.

Referring to FIG. 20E collet-drive system 1500 is indicated in a device exchange state (second alternate state). In the device exchange state collet 1504 unpinches EMD 1502, first drive module 1508 and second drive module 1510 move toward one another reducing their separation distance (the same as the collet lock state), the spline teeth of first engagement portion 1514 mesh with spline teeth of second engagement portion 1518 (that is, they are engaged, although not fully), and drive tire 1558 and idler tire 1568 are separated from one another and do not grip EMD 1502. In the exchange state, just as in the collet lock state, rocker distal post 1575 is in contact with the inside face of first lip 1580 and straddle rocker 1570 rotates moving idler tire 1568 toward drive tire 1558 but the tires do not grip EMD 1502.

In the exchange state collet 1504 unpinches EMD 1502 by rotation of capstan bevel gear 1526 that meshes and rotates driven bevel gear 1524 that rotates collet second member 1516 relative to collet first member 1512. Note that collet first member 1512 is locked (does not move) due to engagement of spline teeth of first engagement portion 1514 with spline teeth of second engagement portion 1518 that does not move. With collet 1504 in an unpinched state EMD 1502 can be removed. In one embodiment EMD 1502 can be removed by side or radial unloading with alignment of a collet slit 1582 in collet 1504 and a collet engagement member slit 1584 in collet engagement member 1506. In one embodiment EMD 1502 can be removed by axial unloading.

Referring to FIG. 20A collet slit 1582 extends longitudinally from an outer circumferential surface and extends radially through collet 1504 to its center line and collet engagement member slit 1584 extends longitudinally from an outer surface circumferential and extends radially through collet engagement member 1506 to its center line. In one embodiment slits 1582 and 1584 have parallel walls. In one embodiment slits 1582 and 1584 have nonparallel walls, such as v-shaped walls with the vertex toward the radial center. In one embodiment slits 1582 and 1584 have lead-in chamfers at the outer surface. In one embodiment slits 1582 and 1584 have no chamfers at the outer surface.

Referring to FIG. 20F collet-drive system 1500 is indicated in a collet pinched-tire grip state (third state). In the collet pinched-tire grip state collet 1504 pinches EMD 1502, first drive module 1508 and second drive module 1510 move toward one another to their smallest separation distance (for example, second drive module 1510 moves toward a fixed first drive module 1508), the spline teeth of first engagement portion 1514 fully mesh with spline teeth of second engagement portion 1518 (that is, they are engaged fully), and drive tire 1558 and idler tire 1568 are not separated and grip EMD 1502. In the collet pinched-tire grip state rocker distal post 1575 is in contact with the inside face of second lip 1581 and straddle rocker 1570 rotates moving idler tire 1568 into drive tire 1558 such that the tires grip EMD 1502.

Referring to FIG. 20G collet-drive system 1500 is indicated in a tire driving state (fourth state). In the tire driving state collet 1504 unpinches EMD 1502, first drive module 1508 and second drive module 1510 move toward one another to their smallest separation distance (for example, second drive module 1510 moves toward a fixed first drive module 1508), the spline teeth of first engagement portion 1514 fully mesh with spline teeth of second engagement portion 1518 (that is, they are engaged fully), and drive tire 1558 and idler tire 1568 are not separated and grip EMD 1502. In the tire driving state, as in the collet pinched-tire grip state, rocker distal post 1575 is in contact with the inside face of second lip 1581 and straddle rocker 1570 rotates moving idler tire 1568 into drive tire 1558 such that the tires grip EMD 1502.

In the tire driving state collet 1504 unpinches EMD 1502 by rotation of capstan bevel gear 1526 that meshes and rotates driven bevel gear 1524 that rotates collet second member 1516 relative to collet first member 1512. Note that collet first member 1512 is locked (does not move) due to engagement of spline teeth of first engagement portion 1514 with spline teeth of second engagement portion 1518 that does not move. With collet 1504 in an unpinched state EMD 1502 can be translated by rotation of drive tire 1558 gripping EMD 1502 against idler tire 1568.

Collet drive system 1500 operates in a reset mode or in an exchange mode. In the reset mode the sequence for operation is driving state (first state), collet lock state (second state), collet pinched-tire grip state (third state), tire driving state (fourth state), collet pinched-tire grip state (third state), collet lock state (second state), and back to driving state (first state). In the exchange mode the sequence of operation is driving state (first state), collet lock state (second state), device exchange state (second alternate state), collet lock state (second state), and back to driving state (first state).

Collet-drive system 1500 incorporates a collet 1504. To minimize the amount of actuation required collet-drive system 1500 is designed to lock half of collet 1504, preventing rotational motion of this half, while providing a rotational degree of freedom to half of collet 1504 for unpinching and pinching of EMD 1502. There are multiple ways to lock half of collet 1504. The term lock refers to maintaining a component stationary and fixed relative to the patient. If the component is stationary relative to the patient bed rail then for the purposes herein the component is stationary and fixed relative to the patient. One embodiment includes engaging splines. One embodiment includes inserting a locking pin in a hole. One embodiment includes inserting a key in a keyway. One embodiment includes means for mechanical interference that prevent rotation.

In one embodiment, EMD 1502 is unpinched and then after EMD is unpinched, the various components are moved to a homing position to allow for removal of the EMD from the device through aligned slots.

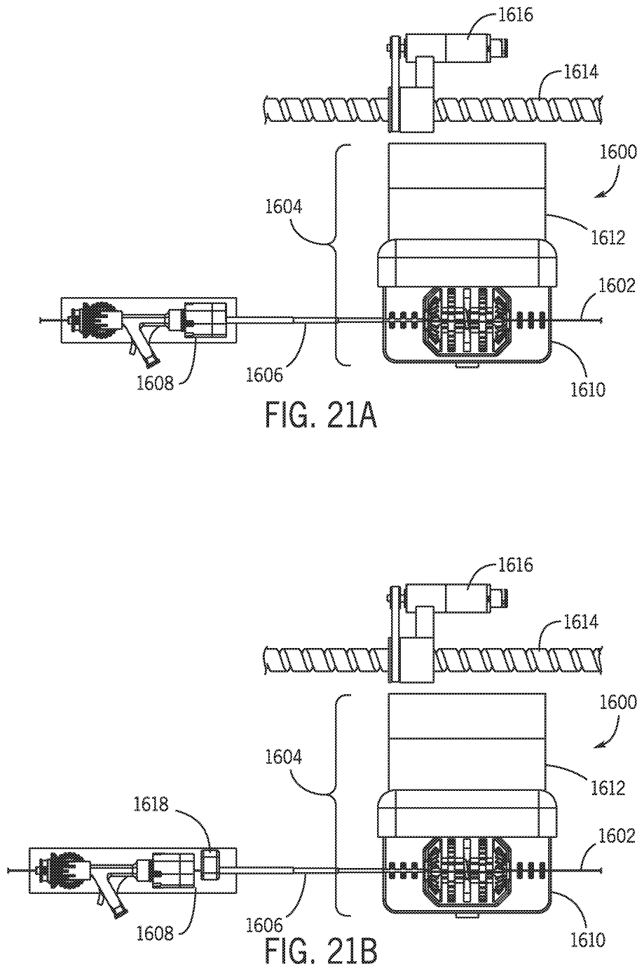

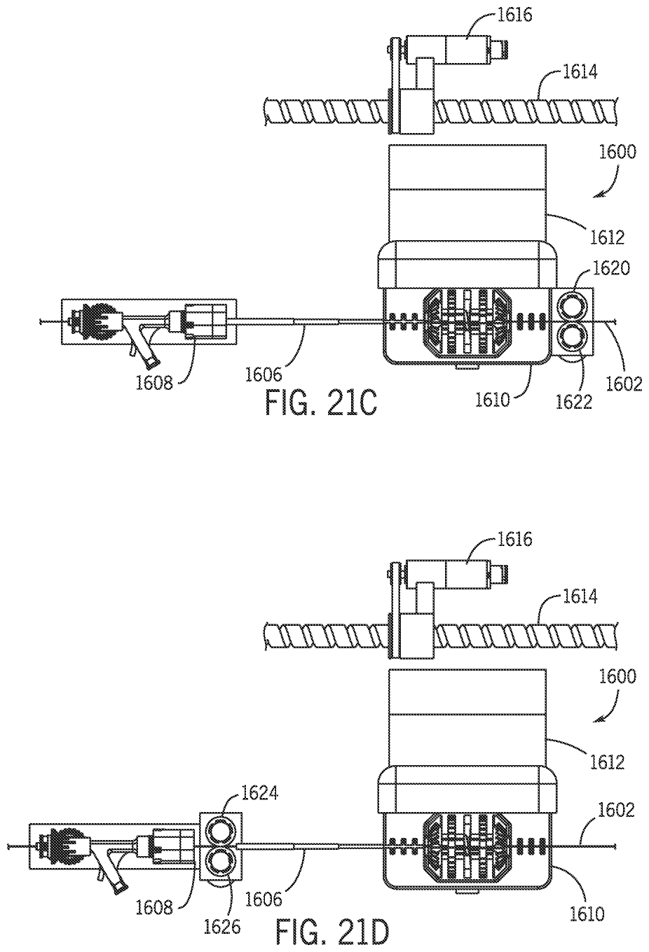

Referring to FIG. 21A a “collet-drive system” 1600 that can rotate, translate, and pinch an EMD 1602 includes a device drive 1604, an EMD support 1606, and a y-connector assembly 1608. Device drive 1604 includes a cassette 1610 and a drive module 1612.

Drive module 1612 translates longitudinally relative to a fixed lead screw 1614 (identified as reference 76 in FIG. 3) and is driven by an actuator 1616 (identified as translation motor 64 in FIG. 3.) In one embodiment lead screw 1614 is a ball screw. In one embodiment actuator 1616 is a motor powered by electrical, pneumatic, hydraulic, or other means.

Referring to FIG. 21A collet-drive system 1600 is connected to the overall robotic system 24. In particular, the connection of lead screw 1614, actuator 1616, and drive module 1612 to the overall robotic system is illustrated.

In one embodiment translation of drive module 1612 is accomplished as described for the drive modules of FIG. 20A. (Note that in FIGS. 21A, 21B, 21C, and 21D some components connecting drive module 1612 to the actuation system for translation are not shown.)

Referring to FIGS. 21A, 21B, 21C, and 21D collet drive system 1600 can pinch and unpinch EMD 1602, rotate clockwise and rotate counterclockwise EMD 1602, and advance and withdraw (that is, translate forward and back) EMD 1602. In one embodiment cassette 1610 is the same as cassette 922 of FIG. 12A and includes a double-bevel collet and rotational drive for pinching and unpinching EMD 1602 and for rotating EMD 1602 in a pinched collet. In other words, collet drive system 1600 includes a collet such as collet 964 of FIG. 12D that can pinch and unpinch EMD 1602.

EMD support 1606 is a constraint preventing EMD 1602 from buckling as EMD 1602 is advanced distally. In one embodiment EMD support 1606 is a system of telescoping sections with inner diameters larger than the diameter of EMD 1602. In one embodiment EMD support 1606 is a track that allows the device to be radially loaded. In one embodiment EMD support 1606 is a tube. In one embodiment EMD support 1606 is any system that prevents EMD 1602 from buckling or bending when advancing.

Referring to FIG. 21B collet-drive system 1600 of FIG. 21A is indicated with a holding clamp 1618 as part of the y-connector assembly 1608. EMD support 1606 is used between y-connector assembly 1608 and cassette 1610. Holding clamp 1618 is a safety mechanism so EMD 1602 does not move when resetting. In one embodiment holding clamp 1618 includes two opposing blocks that can be in a clamped state that constrains the position of EMD 1602 relative to the y-connector assembly 1608 or in an unclamped state that does not constrain the position of EMD 1602 meaning that it is free to move. In one embodiment holding clamp 1618 includes two opposing pads that can be in a clamped state or in an unclamped state. The actuation system for engaging (clamping) and disengaging (unengaging) holding clamp 1618 is not shown.

Referring to FIG. 21C collet-drive system 1600 of FIG. 21A is indicated with a first tire 1620 and a second tire 1622 that oppose each other and press together to grip EMD 1602. First tire 1620 and second tire 1622 are located proximal to cassette 1610. EMD support 1606 is used between y-connector assembly 1608 and cassette 1610. The actuation system for moving first tire 1620 and second tire 1622 toward and away from each other is not shown. Rotation of first tire 1620 and second tire 1622 at the same speed and opposing directions allows EMD 1602 to be translated at higher speed than can be accomplished using a lead screw drive. The use of first tire 1620 and second tire 1622 offers fast transverse of EMD 1602 as well as unlimited travel. In one embodiment the translational speed of device drive 1604 can be synchronized with the rotational speeds of first tire 1620 and second tire 1622 such that EMD 1602 does not move. The method to reset using the collet drive system of 21C involves gripping EMD 1602 between tires 1620 and 1622. Collet 964 is then unpinched freeing EMD 1602 from being fixed thereto. Drive module 1612 is then translated in a first direction while rotating tires 1620 and 1622 to maintain EMD in a fixed location relative to the earth and/or patient. Once the drive module 1612 is moved to the new desired location the collet is actuated to pinch the EMD 1602 thereto and tires 1620 and 1622 ungrip EMD 1602. In this manner the collet drive module is reset for continued travel. In one embodiment reset occurs when translating EMD 1602 in a distal direction once drive module cannot be moved any further in the distal direction. To reset the drive module to continue driving EMD 1602 in the distal direction, drive module 1612 is moved in the proximal direction to a reset position. During translational reset for continued distal driving the first direction noted above is the proximal direction. As the drive module 1612 is moving proximal in order to maintain EMD 1602 stationary relative to the patient, tires 1620 and 1622 rotate in a manner to maintain EMD 1602 to compensate for the proximal movement of drive module 1612.

Referring to FIG. 21D collet-drive system 1600 of FIG. 21A is indicated with a third tire 1624 and a fourth tire 1626 that oppose each other and press together to grip EMD 1602. Third tire 1624 and fourth tire 1626 are located proximal to y-connector assembly 1608 and distal to EMD support 1606. EMD support 1606 is used between y-connector assembly 1608 and cassette 1610. Third tire 1624 and fourth tire 1626 replace the holding clamp 1618 of FIG. 21B. The actuation system for moving third tire 1624 and fourth tire 1626 toward and away from each other is not shown.

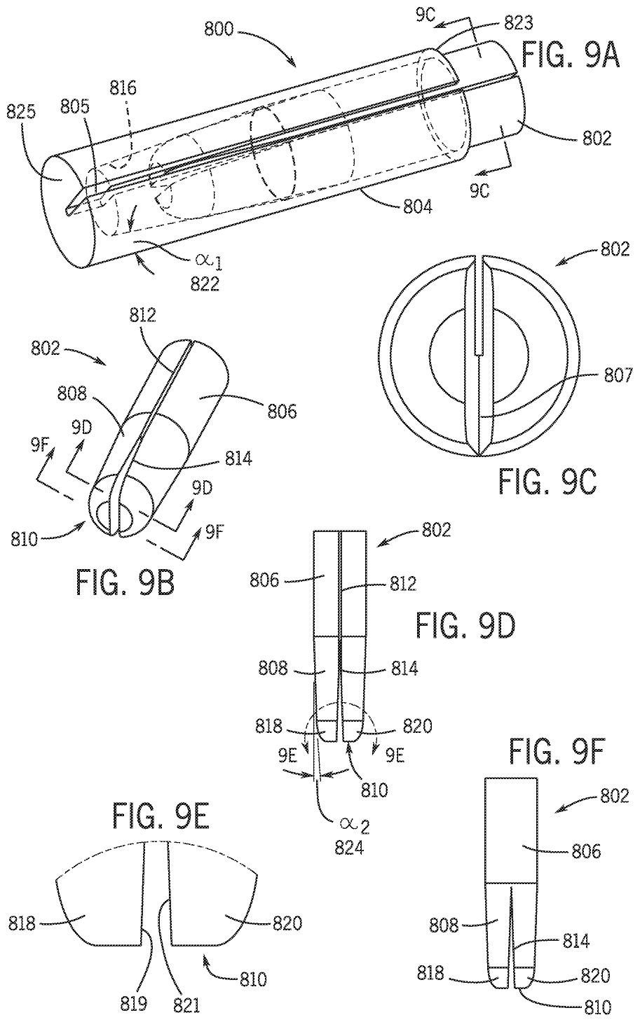

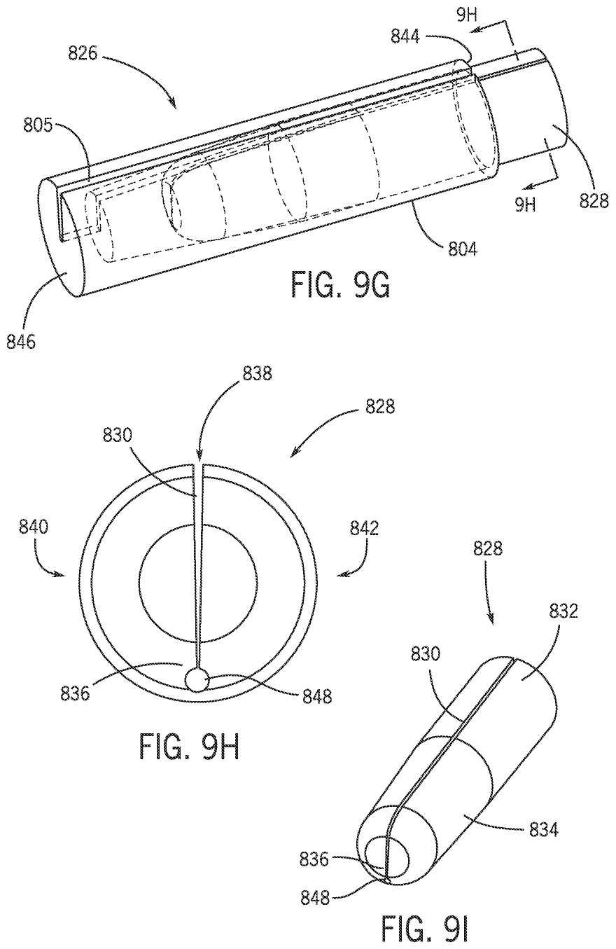

COLLETS: A number of collet designs are provided herein that may be used in the robotic systems described. Referring to FIG. 9A a collet 800 releasably engages an EMD (not shown). Collet 800 includes an inner member 802 that is movably positioned in a distal or proximal direction within a receiving sleeve with tapered cavity 816 of outer member 804. Outer member 804 has a longitudinal slit 805 extending from an outer surface of the outer member and terminating at its radial center. In one embodiment the walls of slit 805 are parallel. In one embodiment the walls of slit 805 are nonparallel, such as v-shaped walls with a vertex toward the radial center. In one embodiment there is a lead-in chamfer at the outer surface of slit 805. In one embodiment there is no chamfer at the outer surface of slit 805.

Referring to FIG. 9B inner member 802 includes a first section 806 having a generally constant radius and a second tapered section 808 that extends from first section 806 in a frusto-conical manner such that the diameter of the second section continuously decreases from a region immediately adjacent the first section to a distal free end 810 of the second section 808, where the distal free end 810 of the second section 808 is further from the region of the second section immediately adjacent the first section 806. In one embodiment the length of first section 806 and the length of second section 808 are the same. In one embodiment the length of first section 806 is greater than the length of second section 808. In one embodiment the length of first section 806 is less than the length of second section 808.

First section 806 has a longitudinal slit 812 extending from an outer surface of the first section and terminating at a radial center of the inner member 802. Second tapered section 808 has a longitudinal slit 814 extending through the entire second section 808 from a portion of the outer surface of the second section in line with the slit 812 in the first section 806 to a portion of the outer surface of the second section 180 degrees from the first outer surface region. The second slit 814 defines a first plane and a second plane at an angle to the first plane. In one embodiment slit the walls of slit 812 are parallel and the walls of slit 814 are nonparallel. In one embodiment the walls of slit 812 and slit 814 are parallel. In one embodiment the walls of slit 812 and slit 814 are nonparallel.

Referring to FIG. 9B two cross-sections are indicated in FIG. 9D and FIG. 9F. In one embodiment slit 812 exists in the top portion of inner member 802 and slit 812 does not exist in the bottom portion of inner member 802.

Referring to FIG. 9C first section 806 and second section 808 are connected along a connecting portion at the lower portion of inner member 802 at seam line 807.

Referring to FIG. 9A movement of inner member 802 from a first end 823 of outer member cavity toward the tapered end 825 of outer member cavity causes the two sections 818 and 820 to move toward one another to pinch the EMD (not shown). Similarly, movement of the inner member 802 in a direction from the second tapered end 825 of the outer member 804 toward the first open end 823 of the outer member results in the two section 818 and 820 to move away from one another pivoting about a line through the seam 807.

Referring to FIG. 9D in one embodiment contact between inner member 802 and outer member 804 occurs between the inner circumferential surface of the tapered cavity 816 and the outer circumferential surface of the distal end 810 of the second section 808. In one embodiment this contact is limited to 1 to 5 mm longitudinal distance. In one embodiment this contact is larger than 5 mm longitudinal distance.

Referring to FIG. 9D, FIG. 9E, and FIG. 9F the two portions 818 and 820 of the second section 808 of the inner member 802 are increasingly separated in the direction of the distal end 810 in a “normally opened” unloaded configuration.

In operation, translational movement of the inner member 802 into the tapered cavity 816 of outer member 804 forces the two portions 818 and 820 of the second section or portion 808 to move toward each other thereby causing the two facing surfaces 819 and 821 of portions 818 and 820, respectively, to move toward each other to pinch the EMD. As inner member 802 moves distally into outer member 804 compressive forces due to contact between inner member 802 and outer member 804 (that occur between the inner circumferential surface of the tapered cavity 816 and the outer circumferential surface of the distal end of inner second section 808) act on the two sections of inner member second section 808. These forces overcome the inherent compliance of the two sections of inner member second section 808 resulting in the two facing surfaces 819 and 821 of portions 818 and 820, respectively, moving toward one another in a loaded configuration.

In one embodiment in the loading configuration the inner surfaces 819 and 821 of the second section 808 of inner member 802 contact the EMD first at the distal free end 810 and then progressively continue to contact the EMD proximally in the slit 814 of inner member tapered second section 808.

To move inner member 802 into outer member 804 requires an external driving force in the distal direction applied to inner member 802 from an operator or robotic system (not shown). In one embodiment the external driving force in the distal direction is applied to the proximal end of inner member 802. In one embodiment inner member is moved relative to outer member by rotating one of the inner member 802 and outer member 804 with a rotational input that engages a screw member to translate the inner member 802 relative to outer member 804 linearly along the longitudinal axes of the collet.

To increasingly move inner member 802 distally into outer member 804 requires an increasing external driving force to overcome the increasing compliance force (to increasingly move the two facing surfaces 819 and 821 of portions 818 and 820, respectively, to move toward one another) and to overcome the increasing friction force (as a result of increasing contact between the inner circumferential surface of the tapered cavity 816 and the outer circumferential surface of the distal end of second section 808).

The loaded configuration becomes a locked configuration when the two facing surfaces 819 and 821 of portions 818 and 820, respectively, pinch down on the EMD such that the EMD cannot move. In the locked configuration no external driving force is needed. Friction forces (due to contact between the inner circumferential surface of the tapered cavity 816 and the outer circumferential surface of the distal end of second section 808) maintain the collet 800 in the locked configuration. In other words, in the locked configuration inner member 802 is locked with outer member 804 due to friction.

In operation, translational movement of the inner member 802 away from the tapered cavity 816 of outer member 804, that is when inner member 802 is withdrawn with respect to outer member 804, separates the two portions 818 and 820 of the second section or portion 808 from one another thereby causing the two facing surfaces 819 and 821 of portions 818 and 820, respectively, to move away from one another to unpinch the EMD. When inner member 802 is withdrawn from outer member 804 the inherent compliance of the two sections of inner member second section 808 restores the two facing surfaces 819 and 821 of portions 818 and 820, respectively, to their normally opened unloaded configuration.

To move inner member 802 away from outer member 804 requires an external driving force in the proximal direction applied to inner member 802 from an operator or robotic system (not shown). The external driving force in the proximal direction must overcome the friction force keeping the collet mechanism 800 in the locked configuration. In one embodiment the external driving force is applied to the proximal end of inner member 802.

In one embodiment the two sections of inner member second section 808 are connected by a living hinge with spring properties that force the two sections away from one another as the inner member is moved toward the open end of the outer member. In one embodiment a separate spring operates to bias the two sections apart.

In one embodiment the outer surface of inner member tapered second section 808 has smooth walls. In one embodiment the outer surface of inner member tapered second section 808 has walls that are not smooth, for example, one or more concave pockets or wells appear on the outer surface. Designs with non-smooth walls allow for nonuniform and generally lower inherent compliance of the two sections of inner member tapered second section 808 in comparison to designs with smooth walls.

In one embodiment the inner member 802 is made of a moldable plastic. In one embodiment the inner surfaces 819 and 821 of the second section 808 of inner member 802 include an elastomeric or other deformable or compliant material that deforms about the EMD during pinching and in the locked configuration.

In one embodiment an EMD is radially loaded through outer member slit 805 and inner member slit 812 and slit 814 when slits 805, 812, and 814 are aligned. The radial loading allows a user to place an EMD into the center of the collet without having to thread a free end of the EMD through a first end 823. Rather a portion of the EMD between a first end and a second end of the EMD is placed directly into the radial center of the collet through aligned slits 805, 812 and 814. In radial loading a first terminal end of the EMD remains distal the distal end of the collet and the second opposed terminal end of the EMD remains proximal the proximal end of the collet while the portion of the EMD intermediate first end and second end of the EMD is inserted through slits 805, 812, and 814 to the radial center of the collet. Loading an EMD described in this paragraph is referred to herein as side loading or radial loading.

Referring to FIG. 9A and FIG. 19D the angle α1 822 of the taper of the inner cavity 816 of the outer member 804 is greater than the angle α2 824 of the taper of the outer surface of the second section 814 of the inner member thereby forcing the two portions 818 and 820 toward one another as the inner member is moved into the cavity 816 in a direction toward the second end of the outer member 804.

Referring to FIG. 9C in one embodiment of inner member 802 the longitudinal slit 812 that extends from the outer surface of the first section 806 terminates at the central longitudinal axis of the inner member 802. In one embodiment of inner member 802 the longitudinal slit 812 that extends from the outer surface of the first section 806 terminates off the central longitudinal axis of the inner member 802.

In one embodiment first portion 818 and second portion of second section 808 defines two cantilevered portions that extend from inner member first section. Cantilevered portions 818 and 820 have a varying spring forces along their respective longitudinal length such that the surfaces 819 and 821 that contact the EMD positioned therebetween conform well to the EMD to keep pressure applied to the EMD low and spread out along the surfaces 819 and 821. The spring force applied to the EMD can be made to vary by changing the cross-sectional thickness of the cantilevered portions 818 and 820 along the longitudinal axis of collet 800

Collet 800 offers the feature of increased stiffness for greater release force with full slit 814 in second section 808 of inner member 802 and partial slit 812 in first section 806 in inner member 802.

Referring to FIG. 9G a collet 826 has an inner member 828 and an outer member 804. Outer member 804 has the same geometry as outer member 804 described above and shown in FIG. 9A. The principle of operation of the collet 826 is similar to that of collet 800 of FIG. 9A.

Referring to FIG. 9H and FIG. 9I inner member 828 has a longitudinal slit 830 that extends from a region 832 on outer surface 834 of the inner member 828 and extends through the inner member 828 terminating in a region 836 proximate but not through the outer surface approximately 180 degrees from the opening 838 of the slit 830.

Referring to FIG. 9H longitudinal slit 830 forms two approximately semicircular cross-sectional sections, a first section 840 and a second section 842, of inner member 828 that pivot about a region 836 at which slit 830 terminates. In one embodiment slit 830 creates facing parallel walls from sections 840 and 842 in the unloaded configuration, that is, the unpinched state. In one embodiment slit 830 creates facing nonparallel walls, for example, such as v-shaped walls, from sections 840 and 842 in the unloaded configuration, that is, the unpinched state. In one embodiment a stress relief 848 is used at the region of the inner member proximate the bottom of the slit 830 to minimize the effects of stress concentration and thereby minimize the possibility of failure. In one embodiment other means for stress relief are employed at the region of the inner member proximate the bottom of the slit 830.

Referring to FIG. 9G translational movement of inner member 828 from a first end 844 of outer member cavity toward the tapered end 846 of outer member cavity causes the first section 840 and second section 842 of inner member 828 to move toward one another to pinch the EMD (not shown). Similarly, translational movement of inner member 828 in a direction from the second tapered end 846 of outer member 804 toward the first open end 844 of the outer member results in the first section 840 and second section 842 of inner member 828 to move away from one another pivoting about a line through the longitudinal slit 838 to unpinch the EMD (not shown).

In one embodiment the region of the inner member 836 proximate the bottom of slit 830 is a living hinge with spring properties that force the two sections away from one another as the inner member is moved toward the open end of the outer member. In one embodiment a separate spring operates to bias the two sections 838 and 840 apart.

Friction forces (due to contact between the inner circumferential surface of the tapered cavity of outer member 804 and the outer circumferential surface of the distal end of second section 834) maintain the collet 826 in the locked configuration. In other words, in the locked configuration inner member 828 is locked with outer member 804 due to friction.

Based on the dimension and angle of longitudinal slit 830 that forms two sections, a first section 840 and a second section 842, of inner member 828, the collet accommodates a larger range of diameters of EMDs in comparison to the collet of FIG F2A.

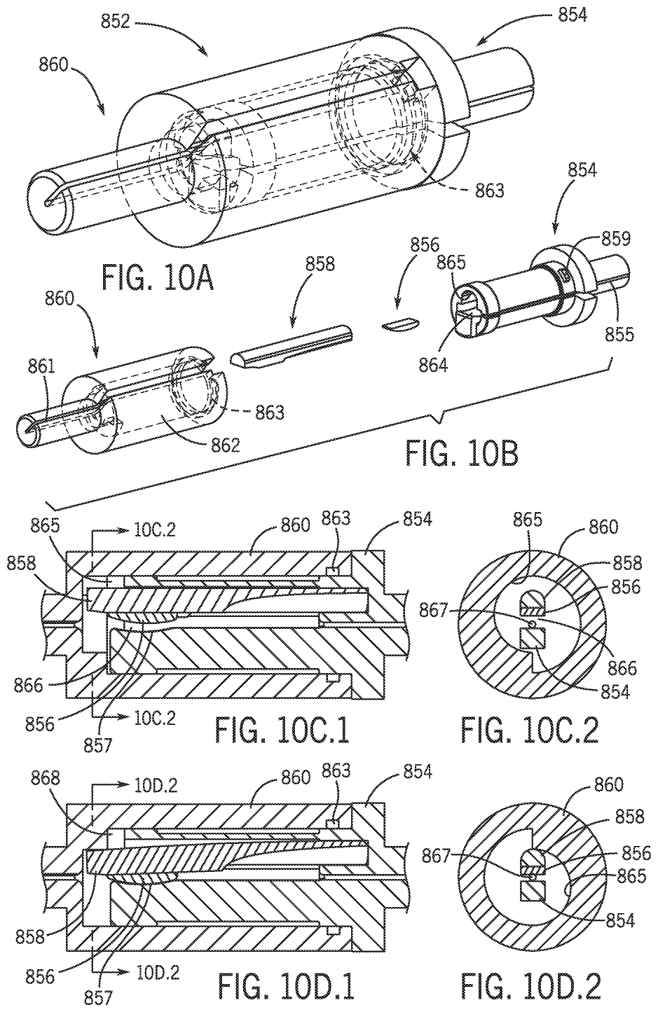

Referring to FIGS. 10A and 10B a collet 852 has an inner member 854, two internal components including a follower pad 856 and a follower finger 858, and an outer member 860. Outer member 860 has a prismatic internal cavity 862 which receives internal components 856 and 858 oriented by an internal cavity 864 of inner member 854. Outer member 860 contains a circumferential retaining channel 863 on the internal surface of the outer member toward its proximal end. Inner member 854 contains a key 859 on the outer surface of inner member that is sized to fit within channel 863. In one embodiment follower pad 856 and follower finger 858 are separate pieces. In one embodiment follower pad 856 and follower finger 858 are integrally connected in one integrated piece. In one embodiment follower pad 856 and follower finger 858 are made of the same material. In one embodiment follower pad 856 and follower finger 858 are made of different materials. For example, in one embodiment follower pad 856 is made of an elastomeric material and follower finger 858 is made of a moldable plastic. In one embodiment follower pad 856 is made of one material. In one embodiment follower pad 856 is made of more than one material, such as a moldable plastic with an elastomeric coating. In one embodiment follower pad 856 has two parallel flat surfaces. In one embodiment follower pad 856 has two nonparallel flat surfaces. In one embodiment follower pad 856 has one flat surface and one curved surface, such as a convex surface.

Inner member 854 has a longitudinal slit 855 along its full length extending from an outer surface of the inner member and terminating at its radial center. Outer member 860 has a longitudinal slit 861 along its full length extending from an outer surface of the outer member and terminating at its radial center. In one embodiment slits 855 and 861 have parallel walls. In one embodiment slits 855 and 861 have nonparallel walls, such as v-shaped walls with its vertex toward the radial center. In one embodiment slits 855 and 861 have lead-in chamfers at the outer surface. In one embodiment slits 855 and 861 have no chamfers at the outer surface.

Referring to FIG. 10C.1 and FIG. 10D.1 diametral cross-sections of the assembled collet 852 in unpinched (open) and pinched (closed) configurations, respectively, are indicated with the configuration dependent on relative angular orientation of inner member 854 with respect to outer member 860 about a longitudinal axis. Referring to FIG. 10C.2 a gap 866 exists between an external surface of follower pad 856 and an internal surface of inner member 854 such that there is no pinching of EMD 867. (EMD 867 is not shown in FIG. 10C.1.) In the default unpinched configuration gap 866 exists due to dimensional geometry of an internal cam 865 of inner member 854 such that there is no contact between internal cam surface 865 and follower finger 858. Referring to FIG. 10D.2 no gap 866 exists between external surface of follower pad 856 and an internal surface of inner member 854 due to the relatively larger dimension of internal cam 865 that contacts follower finger 858 such that there is pinching of EMD 867. (EMD 867 is not shown in FIG. 10D.1.) In the pinched configuration collet 852 remains in a locked state. In one embodiment the internal surface 857 of inner member 854 that receives follower pad 856 in capturing EMD 867 in the pinched configuration is flat. In one embodiment the internal surface 857 of inner member 854 that receives follower pad 856 in capturing EMD 867 in the pinched configuration is concave, for example, having a similar profile to the profile of the outer surface of follower pad 856. In one embodiment inner member 854 is made of one material. For example, in one embodiment inner member 854 is made of moldable plastic. In one embodiment inner member 854 is made of more than one material. For example, in one embodiment the internal surface 857 of inner member 854 that receives follower pad 856 has an elastomeric lining or coating on a moldable plastic inner member 854.

Transition from an unpinched to a pinched configuration or from a pinched to an unpinched configuration requires a user or a drive system to impose relative angular motion between inner member 854 and outer member 860 about the longitudinal axis. In one embodiment rotation of inner member 854 relative to outer member 860 of 90 degrees about the longitudinal axis corresponds to the transition from unpinched to pinched configurations. In one embodiment rotation of inner member 854 relative to outer member 860 of 180 degrees about the longitudinal axis corresponds to the transition from unpinched to pinched configurations. In one embodiment rotation of inner member 854 relative to outer member 860 of an arbitrary value less than 360 degrees about the longitudinal axis corresponds to the transition from unpinched to pinched configurations.

In one embodiment the internal cam 865 is designed to achieve pinching in a clockwise rotation of outer member 860 relative to inner member 854 about the longitudinal axis. In one embodiment the cam is designed to achieve pinching in a counterclockwise rotation of outer member 860 relative to inner member 854 about the longitudinal axis.

In one embodiment the internal cam 865 achieves pinching at a single position in the rotation of inner member 854 relative to outer member 860 about the longitudinal axis. In one embodiment the cam achieves pinching at two or more positions in the rotation of inner member 854 relative to outer member 860 about the longitudinal axis.

In one embodiment the internal cam 865 is designed with a dwell such that relative rotation between inner member 854 and outer member 860 does not result in a change of state, that is, if the collet system 852 is in a pinched configuration it remains in a pinched configuration or if the collet system 852 is in an unpinched configuration it remains in an unpinched configuration. The dwell is achieved by having no change in the radial dimension of the profile of the internal cam 865 over a range of relative rotation between inner member 854 and outer member 860. In one embodiment in a pinched configuration a dwell accommodates for possible errors in the displacement commands to the motors rotationally driving the inner member 854 and the outer member 860 giving some tolerance to errors with the EMD 867 remaining pinched.

In one embodiment cam 865 is designed such that rotation of inner member 854 relative to outer member 860 of 90 degrees about the longitudinal axis maintains the EMD in the pinched configuration. In one embodiment the cam is designed such that rotation of inner member 854 relative to outer member 860 of less than 90 degrees about the longitudinal axis maintains the EMD in the pinched configuration. In one embodiment the cam is designed such that rotation of inner member 854 relative to outer member 860 of more than 90 degrees about the longitudinal axis maintains the EMD in the pinched configuration.

In one embodiment cam 865 is designed such that rotation of inner member 854 relative to outer member 860 of 90 degrees about the longitudinal axis maintains the EMD in the unpinched configuration. In one embodiment the cam is designed such that rotation of inner member 854 relative to outer member 860 of less than 90 degrees about the longitudinal axis maintains the EMD in the unpinched configuration. In one embodiment the cam is designed such that rotation of inner member 854 relative to outer member 860 of more than 90 degrees about the longitudinal axis maintains the EMD in the unpinched configuration.

In the assembled collet 852 key 859 of inner member 854 is retained in channel 863 of outer member 860 allowing for freedom of rotation of inner member 854 relative to outer member 860 and no freedom of translation of rotation of inner member 854 relative to outer member 860. Key 859 captured in channel 863 ensures that inner member 854 and outer member 860 are aligned during assembly such that outer surface of pad 856 of follower finger 858 is positioned longitudinally opposite surface 857 in inner member 854. Key 859 captured in channel 863 prevents both members from being pulled apart when in a pinched or unpinched configuration.

In an initial configuration slit 855 in inner member 854 of collet 852 is aligned with slit 861 in outer member 860 to allow for side or radial loading of EMD as described herein.

Referring to FIG. 11A a collet 868 has an inner member 870, two internal components consisting of a flexure 872 and a collar 874, and an outer member 876.

Inner member 870 has a longitudinal slit 871 along its full length extending from an outer surface of the inner member and terminating at its radial center. Outer member 876 has a longitudinal slit 877 along its full length extending from an outer surface of the outer member and terminating at its radial center. In one embodiment slits 871 and 877 have parallel walls. In one embodiment slits 871 and 877 have nonparallel walls, such as v-shaped walls with its vertex toward the radial center. In one embodiment slits 871 and 877 have lead-in chamfers at the outer surface. In one embodiment slits 871 and 877 have no chamfers at the outer surface.

Referring to FIG. 11B collet 868 is indicated in a fully assembled configuration with slit 871 of inner member 870 and slit 877 of outer member 876 in alignment for side or radial loading of EMD 878.

Referring to FIG. 11C inner member 870 is a single integrated member comprised of four portions with a longitudinal slit 871 from its external surface to its radial center. Starting most proximally, a first portion 882 is a cylindrical section with an internal lumen at its radial center. Distal to first portion 882 a second portion 884 is a cylindrical section with an internal cylindrical cavity. Distal to second portion 884 a third portion 886 is a cylindrical section with external threads 890 and with an internal cylindrical cavity. Distal to third portion 886 a fourth portion 888 is an extension from the third portion 886. In one embodiment the external diameter of second portion 884 is larger than the external diameter of first portion 882. In one embodiment the external diameter of second portion 884 is the same as the external diameter of first portion 882. In one embodiment the external diameter of second portion 884 is smaller than the external diameter of first portion 882. In one embodiment fourth portion 888 is a prismatic extension with a rectangular cross-section perpendicular to a longitudinal axis. In one embodiment fourth portion 888 is a prismatic extension with a non-rectangular cross-section perpendicular to a longitudinal axis. In one embodiment fourth portion 888 is a non-prismatic extension with a non-rectangular cross-section perpendicular to a longitudinal axis.

Outer member 876 is a single integrated member comprised of two portions with a longitudinal slit 877 from its external surface to its radial center. Starting most proximally a first portion 896 is a cylindrical cup section with internal threads 892 at its proximal portion and internal cylindrical cavity at its distal portion. Internal threads 892 mesh with external threads 890 of inner member 870. The cylindrical cavity at the distal portion of first portion 896 receives collar 874. A second portion 898 of outer member 876 is a cylindrical section with an internal lumen at its radial center.

Referring to FIGS. 11C, 11D, and 11E collar 874 is a cylindrical component with a distal portion that has a closed end, a proximal portion that has an internal cavity, and a keyway pocket 875 removed from its outer circumferential surface over its entire length. In one embodiment collar 874 has a closed end with a flush outer circular surface that is perpendicular to the longitudinal axis and an internal cavity. In one embodiment the closed end of collar 874 has arcuate edges to an outer circular surface that is perpendicular to the longitudinal axis with an internal cavity. In one embodiment the closed end of collar 874 has a lip or flange extending from an outer circular surface that is perpendicular to the longitudinal axis with an internal cavity. In one embodiment the internal cavity of collar 874 is centered relative to the center longitudinal axis of its outer diametral plane. In one embodiment the internal cavity of collar 874 is not centered relative to the center longitudinal axis of its outer diametral plane. In one embodiment the internal cavity of collar 874 is rectangular. In one embodiment the internal cavity of collar 874 is cylindrical. In one embodiment the internal cavity of collar 874 is not rectangular or cylindrical. In one embodiment the internal cavity of collar 874 has a corner pocket or well to receive the distal end of flexure 872.

Collar 874 has a longitudinal slit 894 through the collar circumferential wall with a radial slit to its center. In one embodiment slit 894 has parallel walls. In one embodiment slit 894 has nonparallel walls, such as v-shaped walls with its vertex toward the radial center. In one embodiment slit 894 has a lead-in chamfer at the outer surface. In one embodiment slit 894 has no chamfer at the outer surface.

In one embodiment collar 874 is located in the distal portion of the internal cavity of outer member 876 by extension 888 of inner member 870. Extension 888 serves as a mechanical key to ensure that collar 874 rotates with inner member 870 such that the ends of flexure 872 can be squeezed together longitudinally and not be exposed to relative rotation or torque. In other words, the ends of flexure 872 can translate relative to each other and do not rotate relative to each other. Extension 888 is constrained rotationally by a pocket 875 in collar 874 that acts a keyway and is free to translate longitudinally as inner member 870 is rotated relative to outer member 868.

Referring to FIGS. 11A and 11C in one embodiment the proximal portion of the internal cavity of inner member 870 has a corner pocket or well to receive the proximal end of flexure 872. Flexure 872 is a rectangular prism with a length along the axial direction that is longer than either its width or height in a plane perpendicular to the axial direction. In one embodiment flexure 872 is a rectangular prism whose width and height in a plane perpendicular to the axial direction are the same, meaning the flexure 872 has a square cross-section. In one embodiment flexure 872 is a rectangular prism whose width is larger than its height in a plane perpendicular to the axial direction, meaning the flexure 872 has a rectangular cross-section that is wider than it is higher. In one embodiment flexure 872 is a rectangular prism whose width is smaller than its height in a plane perpendicular to the axial direction, meaning the flexure 872 has a rectangular cross-section that is higher than it is wider. In one embodiment flexure 872 is a rectangular prism with sharp edges. In one embodiment flexure 872 is a rectangular prism with rounded edges. In one embodiment flexure 872 is an approximately rectangular prism. In one embodiment flexure 872 is made of a compliant material, such as a moldable plastic or acrylic. Flexure 872 has an elastic bending property that is a function of its geometry (length, width, and height) and its material properties (principally its modulus of elasticity).

In operation pinching EMD 878 is achieved by rotating inner member 870 relative to outer member 876 in a direction about a longitudinal axis that screws together external threads 892 and internal threads 892. As a result, flexure 872 can be made to flex or bend (such that it has a smaller radius of curvature) and an outer surface 873 of flexure 872 (at and near the longitudinal center of the flexure) can be used to pinch EMD 878 against inner surface 880 of inner member 870. The longitudinal distance between the two ends of flexure 872 is determined by rotation of inner member 870 relative to outer member 876 and can be used to vary the amount of flex. As the longitudinal distance between the ends of flexure 872 decreases, the flex or bend of the flexure increases giving the flexure a smaller radius of curvature and a larger lateral distance, defined as the distance perpendicular to the longitudinal axis at the longitudinal center of the flexure between the outer surface 873 of the unflexed flexure 872 and the outer surface 873 of the flexed flexure 872. Since the lateral distance is constrained by the internal cavity, EMD 878 is trapped between outer surface 873 of flexure 872 and internal surface 880 of inner member 870.

In operation unpinching EMD 878 is achieved by rotating inner member 870 relative to outer member 876 in a direction about a longitudinal axis that unscrews external threads 892 and internal threads 892. As a result, flexure 872 can be made to unflex or unbend (such that it has a larger radius of curvature) and outer surface 873 of flexure 872 unpinches EMD 878 from inner surface 880 of inner member 870. The longitudinal distance between the two ends of flexure 872 is determined by rotation of inner member 870 relative to outer member 876 and can be used to vary the amount of flex. As the longitudinal distance between the ends of flexure 872 increases, the flex or bend of the flexure decreases giving the flexure a larger radius of curvature and a smaller lateral distance, defined as the distance perpendicular to the longitudinal axis at the longitudinal center of the flexure between the outer surface 873 of the unflexed flexure 872 and the outer surface 873 of the flexed flexure 872. In the unpinched configuration the lateral distance between the outer surface 873 of flexure 872 and internal surface 880 of inner member 870 is larger than the diameter of EMD 878 such that EMD 878 is free.

In one embodiment the internal surface 880 of inner member 870 that receives flexure 872 in capturing EMD 878 in the pinched configuration is concave, for example, having a similar profile to the profile of the outer surface 873 of flexed flexure 872. This would increase the surface area contacting EMD 878 and can increase the resistive torque on EMD 878 by moving it away from the central axis of rotation. In one embodiment the internal surface 880 of inner member 870 that receives flexure 872 in capturing EMD 878 in the pinched configuration is flat.

In one embodiment inner member 870 is made of one material, for example, moldable plastic. In one embodiment inner member 870 is made of more than one material. For example, in one embodiment the internal surface 880 of inner member 870 that receives flexure 872 in capturing EMD 878 in the pinched configuration has an elastomeric lining or coating on a moldable plastic inner member 870.

In one embodiment flexure 872 is made of one material, for example, moldable plastic. In one embodiment flexure 872 is made of more than one material. For example, in one embodiment flexure 872 has an elastomeric lining or coating on a moldable plastic inner portion.

In one embodiment of collet 868 a single flexure 872 is used. In one embodiment of collet 868 more than one flexure 872 is used. For example, two flexures oriented 180 degrees apart around the central longitudinal axis could be used to pinch and unpinch EMD 878 based on relative rotation of inner member 870 and outer member 876 using the principle described herein.

In an initial configuration slit 871 in inner member 870 of collet 868 is aligned with slit 877 in outer member 876 to allow for side or radial loading of EMD as described herein.

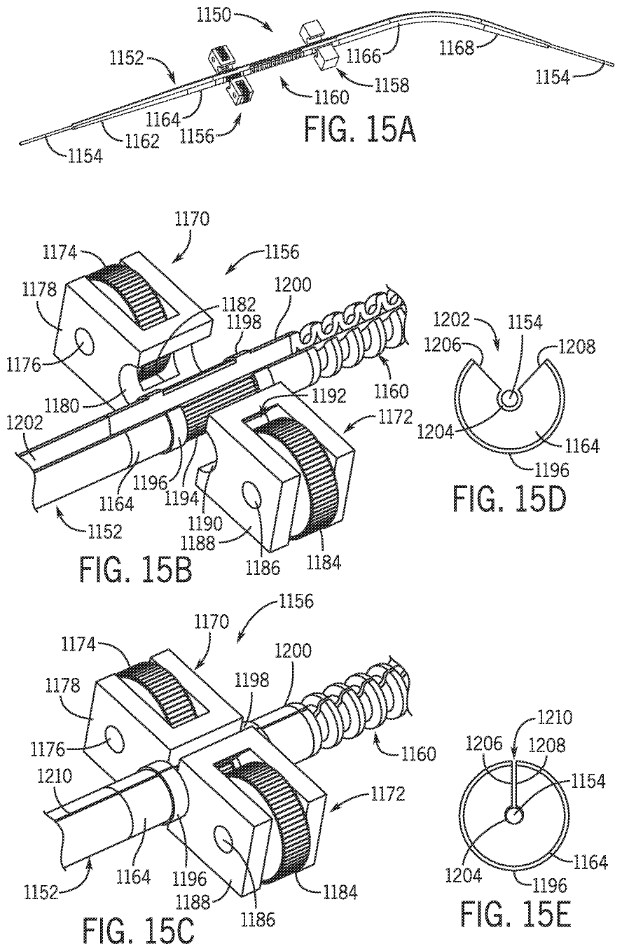

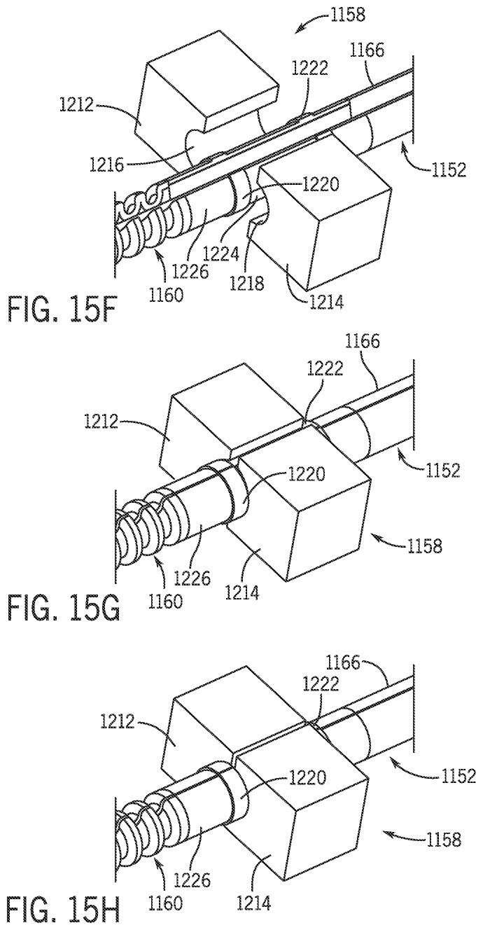

Referring to FIG. 15A a flexible bellows collet-drive system 1150 that can rotate, translate, and pinch an EMD 1154 includes a device retainer 1152, a drive block set 1156, and a holding block set 1158. The device retainer 1152 is a device support that includes a longitudinal section of flexible bellows 1160 that is located between the drive block set 1156 and the holding block set 1158. The flexible bellows 1160 is a device support that allows for translational motion between the drive block set 1156 and the holding block set 1158. In one embodiment the drive block set 1156 is located distal to the flexible bellows 1160 and the holding block set 1158 is located proximal to the flexible bellows 1160. In one embodiment the drive block set 1156 is located proximal to the flexible bellows 1160 and the holding block set 1158 is located distal to the flexible bellows 1160. In one embodiment the device retainer 1152 includes a distal tapered section 1162, a distal constant section 1164, a proximal constant section 1166, and a proximal tapered section 1168. In one embodiment the device retainer 1152 includes a distal constant section 1164 and a proximal constant section 1166, without a distal tapered section 1162 and without a proximal tapered section 1168.

Referring to FIG. 15A the flexible bellows collet-drive system 1150 includes a translational drive system (not shown) that can translate (advance and retract) the drive block set 1156 longitudinally relative to the holding block set 1158.

Referring to FIG. 15B the drive block set 1156 is indicated in an open configuration in which there is no contact between the drive block set 1156 and the device retainer 1152. In one embodiment the drive block set 1156 includes a first drive block assembly 1170 and a second drive block assembly 1172. In one embodiment the drive block set 1156 includes a first drive block assembly 1170 and no second drive block assembly 1172. In one embodiment the design of the first block assembly 1170 and the design of the second drive block assembly 1172 are the same. In one embodiment the design of the first block assembly 1170 and the design of the second drive block assembly 1172 are not the same.

The first drive block assembly 1170 includes a first spur gear 1174, a first spur gear pin 1176, and a first drive block retainer 1178. In one embodiment the first spur gear 1174 rotates about the first spur gear pin 1176 that is held into side walls of the first drive block retainer 1178. In one embodiment the first spur gear 1174 is integrally connected to the first spur gear pin 1176 in the middle of its length, and the ends of the first spur gear pin 1176 on either side of the first spur gear 1174 are supported in holes that act as rotational bearings in the outer walls of the first drive block retainer 1178. In one embodiment the first spur gear 1174 is integrally connected to the first spur gear pin 1176 in the middle of its length, and the ends of the first spur gear pin 1176 on either side of the first spur gear 1174 are supported by rotational bearings that are mounted in the outer walls of the first drive block retainer 1178. In one embodiment the first drive block retainer 1178 includes a first drive block cutout 1180 that exposes a section of first spur gear teeth 1182 of the first spur gear 1174. In one embodiment the first drive block cutout 1180 has a semicircular convex cross-section in a plane transverse to the longitudinal axis.

The second drive block assembly 1172 includes a second spur gear 1184, a second spur gear pin 1186, and a second drive block retainer 1188. In one embodiment the second spur gear 1184 rotates about the second spur gear pin 1186 that is held into side walls of the second drive block retainer 1188. In one embodiment the second spur gear 1184 is integrally connected to the second spur gear pin 1186 in the middle of its length, and the ends of the second spur gear pin 1186 on either side of the second spur gear 1184 are supported in holes that act as rotational bearings in the outer walls of the second drive block retainer 1188. In one embodiment the second spur gear 1184 is integrally connected to the second spur gear pin 1186 in the middle of its length, and the ends of the second spur gear pin 1186 on either side of the second spur gear 1184 are supported by rotational bearings that are mounted in the outer walls of the second drive block retainer 1188. In one embodiment the second drive block retainer 1188 includes a second drive block cutout 1190 that exposes a section of second spur gear teeth 1192 of the second spur gear 1184. In one embodiment the second drive block cutout 1190 has a semicircular convex cross-section in a plane transverse to the longitudinal axis.

The first spur gear 1174 is driven by a first spur gear drive system (not shown) that can rotate the first spur gear 1174 in the clockwise direction or in the counterclockwise direction or not rotate the first spur gear 1174. The second spur gear 1184 is driven by a second spur gear drive system (not shown) that can rotate the second spur gear 1184 in the clockwise direction or in the counterclockwise direction or not rotate the second spur gear 1184. In one embodiment the first spur gear drive system, the second spur gear drive system, and the translational drive system are included in a combined translational-rotational drive system (not shown) that can rotate the first spur gear 1174, rotate the second spur gear 1184, and translate the drive block set 1156 simultaneously. In one embodiment the first spur gear drive system, the second spur gear drive system, and the translational drive system are included in a combined translational-rotational drive system (not shown) that can rotate the first spur gear 1174, rotate the second spur gear 1184, and translate the drive block set 1156 in sequence.

Referring to FIG. 15B the device retainer 1152 includes a geared section 1194 that is a longitudinal section with external spur gear teeth that are oriented along the longitudinal axis of the device retainer 1152 and that are sized to mesh with the teeth of the first spur gear 1174 and the teeth of the second spur gear 1184. The geared section 1194 is located proximal to the distal constant section 1164 and distal to the flexible bellows 1160. The length of the geared section 1194 is larger than the width of the first spur gear 1174 or the width of the second spur gear 1184. In one embodiment the length of the geared section 1194 is ten times the width of the first spur gear 1174 or the width of the second spur gear 1184. In one embodiment the length of the geared section 1194 is less than ten times the width of the first spur gear 1174 or the width of the second spur gear 1184. In one embodiment the length of the geared section 1194 is more than ten times the width of the first spur gear 1174 or the width of the second spur gear 1184. In one embodiment the spur gear teeth of the geared section 1194 are molded into the section of the device retainer 1152.

In one embodiment the device retainer 1152 includes a distal drive collar 1196 and a proximal drive collar 1198. The distal drive collar 1196 is located distal to the geared section 1194 and proximal to the distal constant section 1164. The proximal drive collar 1198 is located proximal to the geared section 1194 and distal to the flexible bellows 1160. The distal drive collar 1196 and the proximal drive collar 1198 are longitudinal sections with flanges or lips that extend outward from the device retainer 1152. In one embodiment the device retainer 1152 includes a first intermediate constant section 1200 that is located distal to the flexible bellows 1160 and proximal to the proximal drive collar 1198.

Referring to FIG. 15B and FIG. 15D in the open configuration of the device retainer 1152 there is an opening 1202 to a central channel 1204 for the EMD 1154. In one embodiment the cross-section of the opening 1202 is a circular sector that is removed from a circular cross-section of the device retainer 1152 that exposes a first face 1206 and a second face 1208. In one embodiment the cross-section of the central channel 1204 is an open circular pocket into which the EMD 1154 can be seated or held. In one embodiment the center of the central channel 1204 is aligned with the center of the device retainer 1152.

Referring to FIG. 15C the drive block set 1156 is indicated in a closed configuration in which the first drive block assembly 1170 and the second drive block assembly 1172 move toward one another each in the direction of the central axis of the device retainer such that the exposed teeth 1182 of the first spur gear 1174 mesh with the teeth of the geared section 1194 and the exposed teeth 1192 of the second spur gear 1184 mesh with the teeth of the geared section 1194. In the closed configuration a part of the outer distal wall of the first drive block retainer 1178 and a part of the outer distal wall of the second drive block retainer 1188 are in contact with or are close to being in contact with the distal drive collar 1196, preventing distal motion of the first drive block assembly 1170 and of the second drive block assembly 1172 relative to the device retainer 1152. In the closed configuration a part of the outer proximal wall of the first drive block retainer 1178 and a part of the outer proximal wall of the second drive block retainer 1188 are in contact with or are close to being in contact with the proximal drive collar 1198, preventing proximal motion of the first drive block assembly 1170 and of the second drive block assembly 1172 relative to the device retainer 1152. As such, in the closed configuration the drive block set 1156, constrained by the distal drive collar 1196 and proximal drive collar 1198, acts like a thrust bearing allowing for rotational motion of the device retainer 1152 and preventing translational of the device retainer 1152 relative to the drive block set 1156. In other words, if there is no translational motion of the drive block set 1156 there is no translational motion of the device retainer 1152. If there is translational motion of the drive block set 1156 (such as advancing and retracting along the longitudinal direction) there is the same corresponding translational motion of the device retainer 1152.

Referring to FIG. 15C and FIG. 15E in the closed configuration of the device retainer 1152 the first face 1206 and the second face 1208 oppose each other and meet at a closed seam 1210 and the central channel 1204 encircles and pinches around the EMD 1154. As such, in the closed configuration the EMD 1154 is pressed upon by the walls of the central cavity 1204 of the device retainer 1152 and cannot move relative to the device retainer 1152. In other words, if there is no translational motion of the device retainer 1152 there is no translational motion of the EMD 1154. If there is translational motion of the device retainer 1152 (such as advancing and retracting along the longitudinal direction) there is the same corresponding translational motion of the EMD 1154. Thus, if there is no translational motion of the drive block set 1156 there is no translational motion of the EMD 1154. If there is translational motion of the drive block set 1156 (such as advancing and retracting along the longitudinal direction) there is the same corresponding translational motion of the EMD 1154.

The drive block set 1156 includes a drive block open-close actuation system (not shown) that moves the first drive block assembly 1170 and the second drive block assembly 1172 toward and away from the device retainer 1152 in a direction transverse to the longitudinal axis. Referring to FIG. 15B the drive block open-close actuation system has moved the first drive block assembly 1170 and the second drive block assembly 1172 to positions in the open configuration. Referring to FIG. 15C the drive block open-close actuation system has moved the first drive block assembly 1170 and the second drive block assembly 1172 to positions in the closed configuration. In one embodiment the drive block open-close actuation system smoothly transitions the first drive block assembly 1170 and the second drive block assembly 1172 from the open configuration to the closed configuration and from the closed configuration to the open configuration. In one embodiment the drive block open-close actuation system discretely positions the first drive block assembly 1170 and the second drive block assembly 1172 in the open configuration or the closed configuration.

Referring to FIG. 15F the holding block set 1158 is indicated in an open configuration in which there is no contact between the first holding block 1212 and the device retainer 1152 and no contact between the second holding block 1214 and the device retainer 1152. In one embodiment the holding block set 1158 includes a first holding block 1212 and a second holding block 1214. In one embodiment the holding block set 1158 includes a first holding block 1212 and no second holding block 1214. In one embodiment the design of the first holding block 1212 and the design of the second holding block 1214 are the same. In one embodiment the design of the first holding block 1212 and the design of the second holding block 1214 are not the same.

In one embodiment the first holding block 1212 includes a first holding block cutout 1216 and the second holding block 1214 includes a second holding block cutout 1218. In one embodiment the first holding block cutout 1216 and the second holding block 1214 each have a semicircular convex cross-section in a plane transverse to the longitudinal axis.

In one embodiment the device retainer 1152 includes a distal holding collar 1220 and a proximal holding collar 1222. The distal holding collar 1220 is located proximal to the flexible bellows 1160 and distal to a constant holding section 1224, which is a longitudinal section of the device retainer 1152 with a constant cross-section transverse to the longitudinal direction. The proximal holding collar 1222 is located distal to the proximal constant section 1166 and distal to the constant holding section 1224. The distal holding collar 1220 and a proximal holding collar 1222 are longitudinal sections with flanges or lips that extend outward from the device retainer 1152. In one embodiment the device retainer 1152 includes a second intermediate constant section 1226 that is located proximal to the flexible bellows 1160 and distal to the distal holding collar 1220. Device retainer 1152 serves as anti-buckling support allowing the collet to have a longer throw than the device buckling distance.

Referring to FIG. 15G the holding block set 1158 is indicated in an intermediate configuration in which the first holding block 1212 and the second holding block 1214 move toward one another each in the direction of the central axis of the device retainer 1152. In the intermediate configuration a part of the outer distal wall of the first holding block 1212 and a part of the outer distal wall of the second holding block 1214 are in contact with or are close to being in contact with the distal holding collar 1220, preventing distal motion of the holding block set 1158 relative to the device retainer 1152. In the intermediate configuration a part of the outer proximal wall of the first holding block 1212 and a part of the outer proximal wall of the second holding block 1214 are in contact with or are close to being in contact with the proximal holding collar 1222, preventing proximal motion of the holding block set 1158 relative to the device retainer 1152. As such, in the intermediate configuration the holding block set 1158, constrained by the distal holding collar 1220 and a proximal holding collar 1222, acts like a thrust bearing allowing for rotational motion of the device retainer 1152 and preventing motion translational of the device retainer 1152 relative to the holding block set 1158. In the intermediate configuration the holding block set 1158 is constrained from translational motion and the EMD 1154 is not fully pinched.

Referring to FIG. 15H the holding block set 1158 is indicated in a closed configuration in which the first holding block 1212 and the second holding block 1214 move toward one another each in the direction of the central axis of the device retainer 1152. In the closed configuration a part of the outer distal wall of the first holding block 1212 and a part of the outer distal wall of the second holding block 1214 are in contact with or are close to being in contact with the distal holding collar 1220, preventing distal motion of the holding block set 1158 relative to the device retainer 1152. In the closed configuration a part of the outer proximal wall of the first holding block 1212 and a part of the outer proximal wall of the second holding block 1214 are in contact with or are close to being in contact with the proximal holding collar 1222, preventing proximal motion of the holding block set 1158 relative to the device retainer 1152. As such, in the closed configuration the holding block set 1158, constrained by distal holding collar 1220 and proximal holding collar 1222, acts like a thrust bearing allowing for rotational motion of the device retainer 1152 and preventing motion translational of the device retainer 1152 relative to the holding block set 1158. In the closed configuration the holding block set 1158 is constrained from translational motion and the EMD 1154 is fully pinched.

The holding block set 1158 includes a holding block actuation system (not shown) that moves the first holding block 1212 and the second holding block 1214 toward and away from the device retainer 1152 in a direction transverse to the longitudinal axis. Referring to FIG. 15F the holding block actuation system has moved the first holding block 1212 and the second holding block 1214 to positions in the open configuration. Referring to FIG. 15G the holding block actuation system has moved the first holding block 1212 and the second holding block 1214 to positions in an intermediate configuration. Referring to FIG. 15H the holding block actuation system has moved the first holding block 1212 and the second holding block 1214 to positions in the closed configuration. In one embodiment the holding block actuation system smoothly transitions the first holding block 1212 and the second holding block 1214 from the open configuration to the intermediate configuration and from the intermediate configuration to the closed configuration and from the closed configuration to the intermediate configuration and from the intermediate configuration to the open configuration. In one embodiment the holding block actuation system discretely positions the first holding block 1212 and the second holding block 1214 in the open configuration, the intermediate configuration, or the closed configuration.

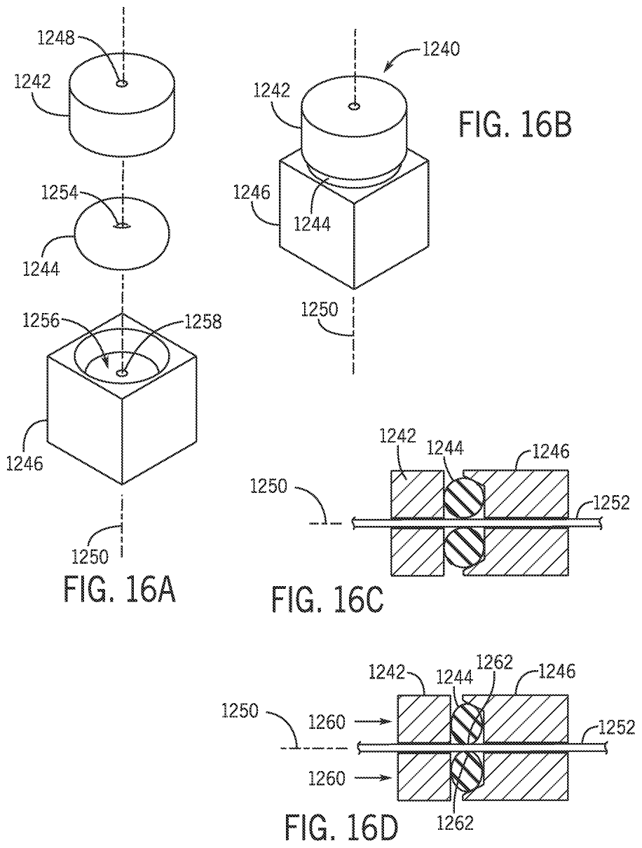

Referring to FIG. 16A and FIG. 16B a compression collet system 1240 includes a plunger 1242, a donut 1244, and a receiver 1246. In one embodiment the plunger 1242 is a rigid right circular cylinder with a central lumen 1248 with the long axis of the cylinder and with the axis of the lumen aligned with an EMD longitudinal axis 1250. In one embodiment the lumen 1248 has a circular cross-section in a plane transverse to the EMD longitudinal axis 1250 with the lumen diameter larger than the outer diameter of an EMD 1252. The donut 1244 is a ring torus made of a compliant material. In one embodiment the donut 1244 is an O-ring. In one embodiment the donut 1244 is made of an elastomeric material. In its rest state, that is, in an unloaded state, the donut 1244 has an internal hole 1254 with the hole diameter larger than the outer diameter of an EMD 1252. The receiver 1246 is a rigid receptacle that includes a well 1256 and an internal lumen 1258 aligned with an EMD longitudinal axis 1250 with the lumen diameter larger than the outer diameter of an EMD 1252. In one embodiment the receiver 1246 is a rectangular prism with a well 1256 on one face with an opening in the shape of a right circular cylinder. In one embodiment the well 1256 has straight walls. In one embodiment the well 1256 has conical walls tapered into the well.

Referring to FIG. 16C and FIG. 16D a plunger actuation system (not shown) translates the plunger 1242 along the EMD longitudinal axis 1250 relative to the receiver 1246 and applies a plunger force 1260.

Referring to FIG. 16C the compression collet system 1240 is indicated in an unloaded configuration in which the plunger 1242 is not pressing against, that is, not applying a plunger force 1260 to, the donut 1244 in the well 1256. As such, the donut 1244 is in its rest state and not deformed, and the EMD 1252 is free to translate relative to the receiver 1246. (The donut has circular cross-sections in the poloidal plane as shown in FIG. 16C.)

Referring to FIG. 16D the compression collet system 1240 is indicated in a loaded configuration in which the plunger 1242 is pressed against the donut 1244 in the well 1256 by a plunger force 1260. As such, the donut 1244 is compressed and deformed (it changes its original shape, for example, from circular cross-sections to elliptical cross-sections in a poloidal plane as shown in FIG. 16D.) In the deformed state, a portion of the deformed surface walls 1262 of the donut hole 1254 pinches around the EMD 1252. As a result, the EMD 1252 is not free to translate relative to the receiver 1246.

In one embodiment a rotational drive system (not shown) rotates (clockwise and counterclockwise) the compression collet system 1240 about the longitudinal axis 1250 of the EMD 1252. In one embodiment a translational drive system (not shown) translates (advances and retracts) the compression collet system 1240 along the longitudinal axis 1250 of the EMD 1252.

In one embodiment the compression collet system 1240 includes slits (not shown) to allow for side or radial loading of EMD 1252.

In one embodiment a collet may include a collet first member and a collet second member that when moved relative to one another pinch and unpinch an EMD. In one embodiment the collet first member and the collet second member may be formed as a single component in which the collet first member and collet second member are compliantly connected. In one non-limiting example collet first member and collet second member may be connected with a living hinge, accordion portion of flexible portions that are movable relative to each other

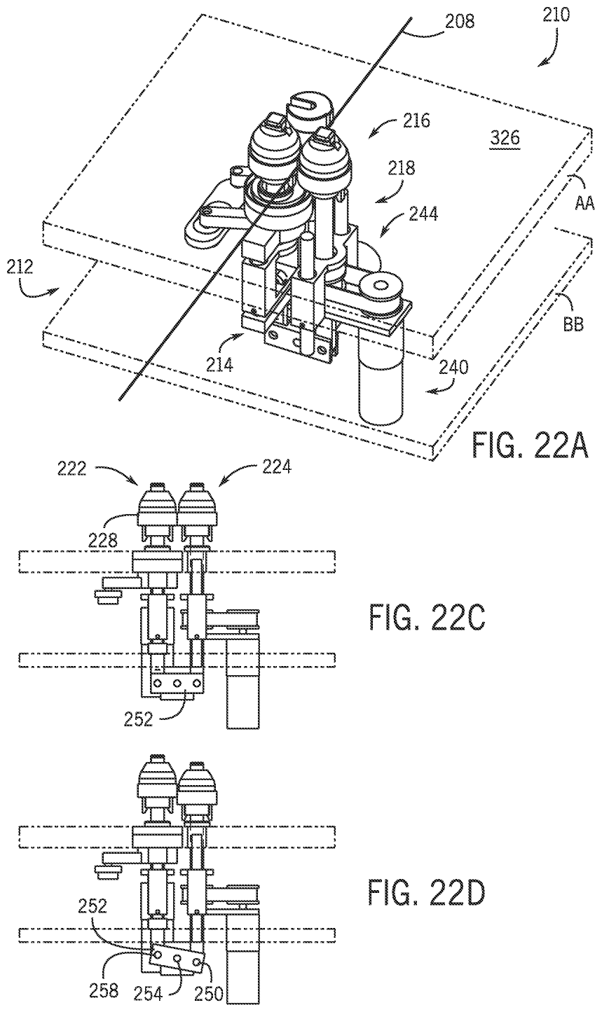

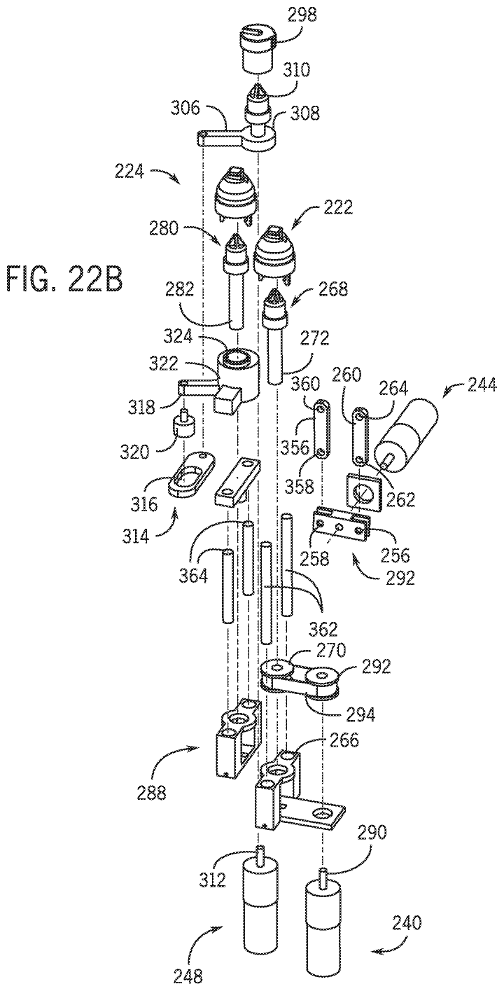

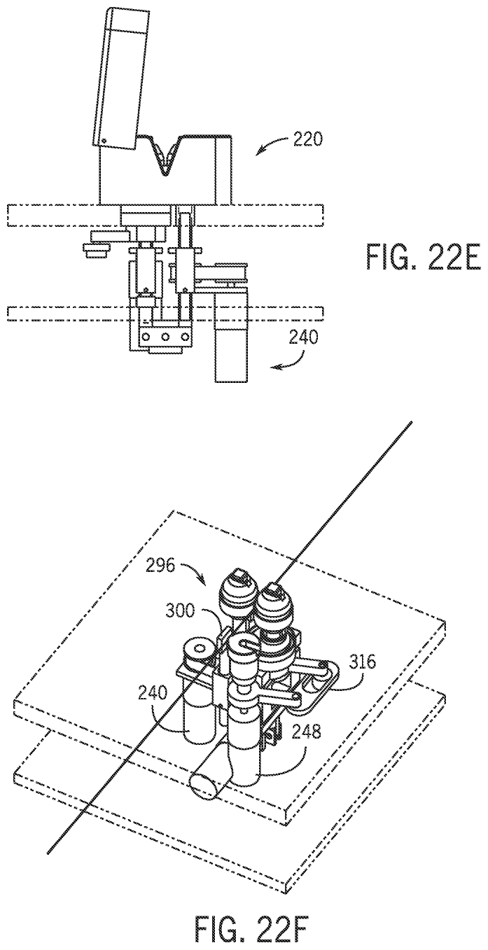

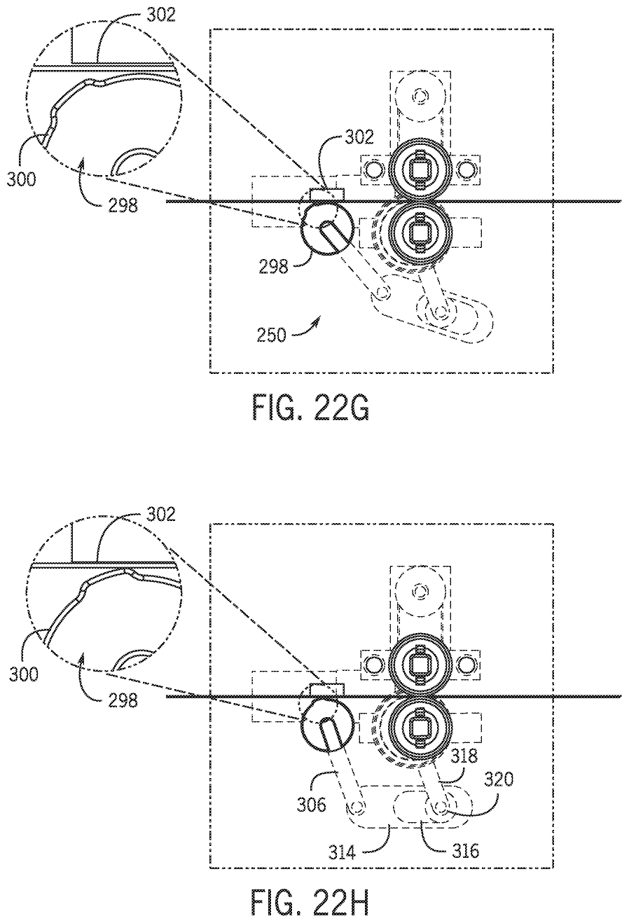

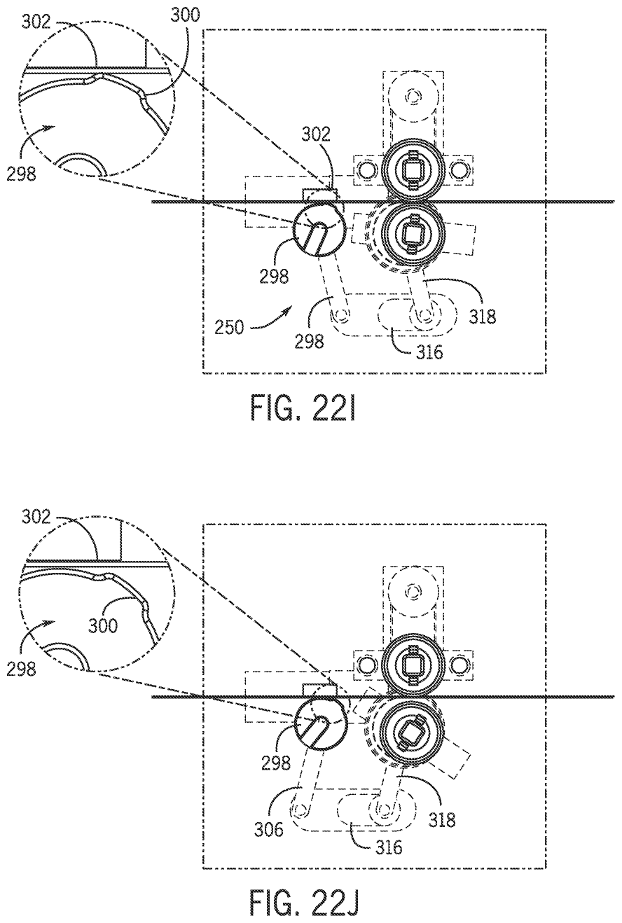

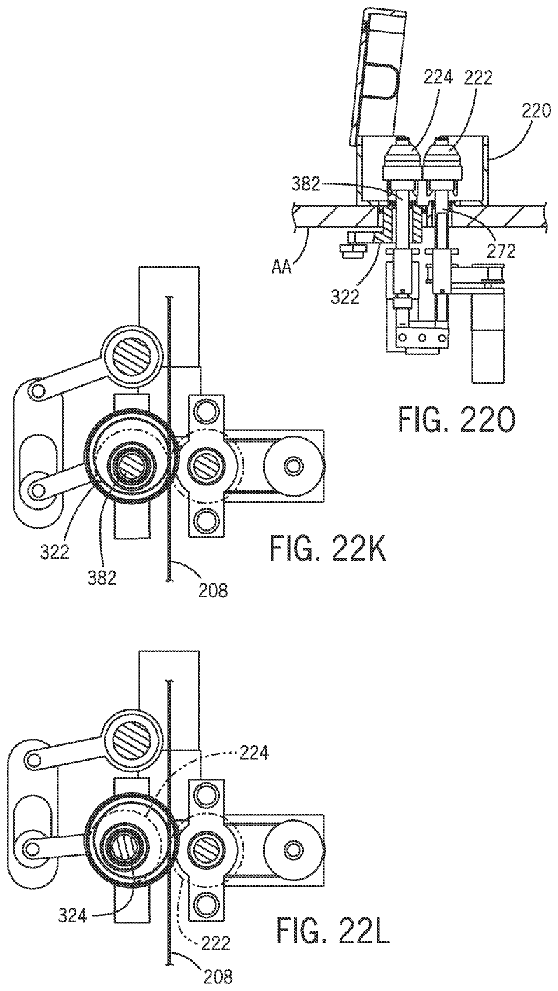

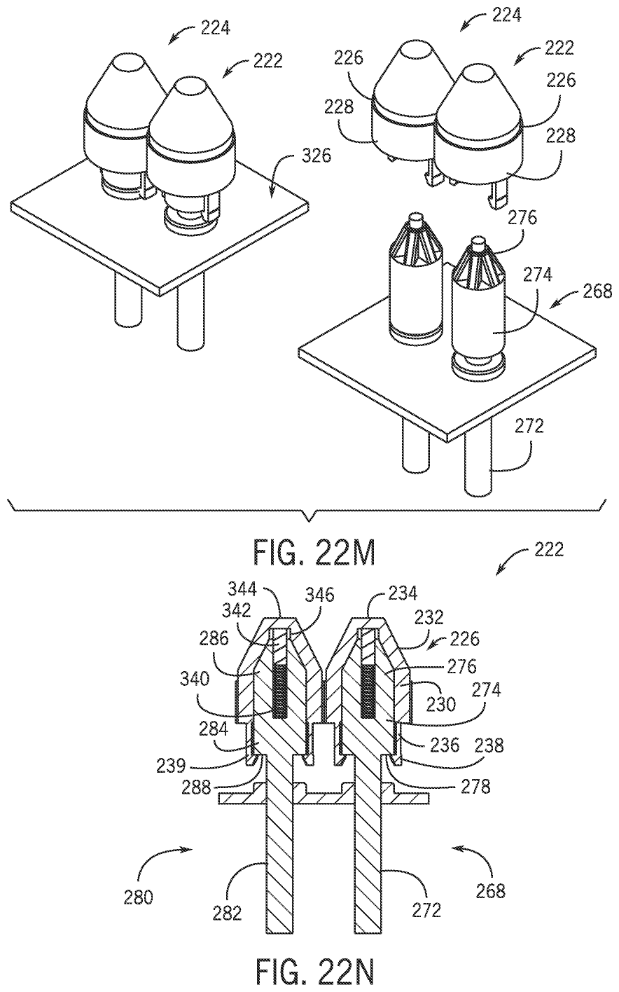

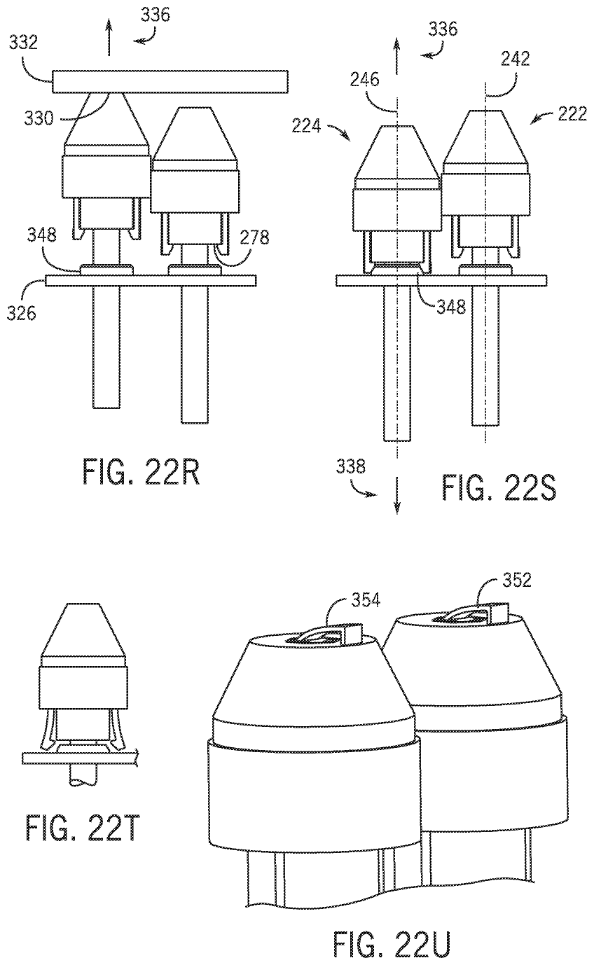

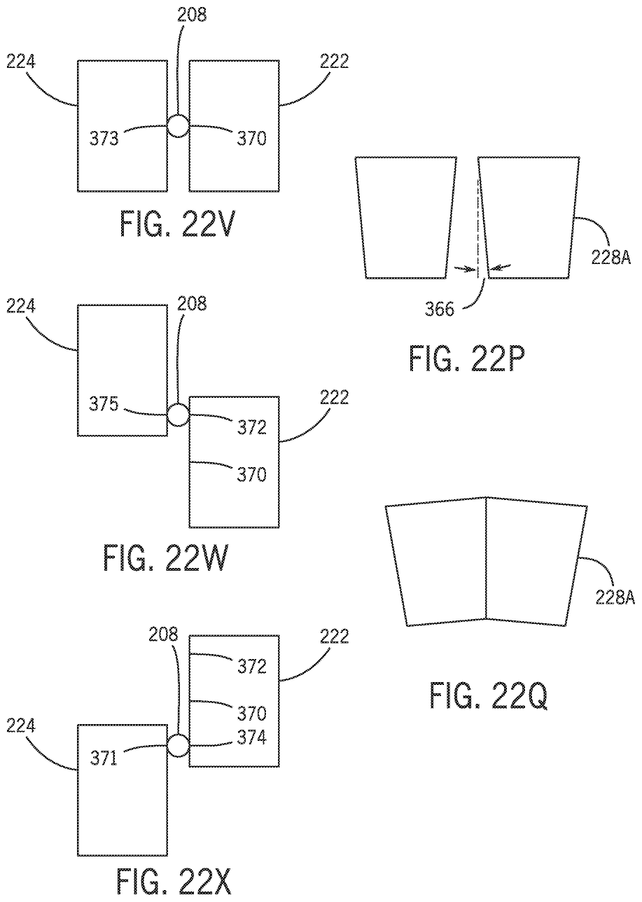

Referring to FIGS. 22A-22X a drive mechanism 210 is a device for the actuation of tires to robotically control the movement of an EMD. In one embodiment drive mechanism has a pair of tires that pinch an EMD between them. In one embodiment, multiple pairs of tires working together including but not limited to 4 pairs in order to increase the grip on the EMD. The tires are rotated about their longitudinal axis to translate the EMD linearly along its longitudinal axis and the tires are moved axially in opposite directions to drive the EMD in rotation about its longitudinal axis. As discussed herein drive mechanism 210 includes three integrated mechanisms to rotate the tires, translate the tires axially and to pinch and unpinch the tires. Additionally, in one embodiment a clamp mechanism operates to clamp and unclamp a portion of the EMD a distance from the pair of tires.

Referring to FIG. 22A a robotic drive system includes a drive module 210 using at least one pair of tire assemblies 222 and 224 rotate EMD 208 about its longitudinal axis, translate EMD 208. along its longitudinal axis and resets the tire assemblies during manipulation of EMD 208. Drive module 210 is controlled by a control system. Drive module 210 includes a first actuator 240 operatively rotating a first shaft 272 and/or a second shaft 282. A second actuator 244 operatively translating first shaft 272 along its longitudinal axis relative to the second shaft 282 between a first position and a second position. The first tire assembly 222 operatively attached to the first shaft 272 and the second tire assembly 224 is operatively attached to second shaft 282. A third actuator 248 operatively moves first tire assembly 222 toward and away from second tire assembly 224 gripping and ungripping EMD 208 along its longitudinal axis from between first tire assembly 222 and the second tire assembly 224. As described in more detail herein, translation of the first shaft 272 relative to the second shaft 282 rotates EMD 208 about the longitudinal axis of the EMD, and rotation of the first shaft 272 and/or second shaft 282 translates EMD 208 along the longitudinal axis of the EMD. The control system provides reset instructions to third actuator 248 to ungrip EMD 208, second actuator 244 to move first tire assembly 222 relative to second tire assembly 224 to a reset position; and to third actuator 248 to grip EMD 208. In one embodiment the reset instructions are provided sequentially.

The reset position is automatically determined as a function of one or more of input device instructions, the offset distance of the two tire assemblies and position of the EMD.

In one embodiment control system provides the reset instructions when the second position reaches a predetermined distance from the first position. Referring to FIG. 22V EMD 208 is positioned at a first position 370 and 373 on first tire assembly 222 and second tire assembly 224 respectively. In one embodiment first positions 370 and 371 are centrally positioned between a first longitudinal end 382, 392 and a second opposing longitudinal end 386, 388 of first tire assembly 222 and second tire assembly 224 respectively. In one embodiment control system provides the reset instructions when the second position reaches a predetermined distance from the first position.

When an operator through a user input provides instructions to rotate EMD 208 about its longitudinal axis in a first direction first tire assembly 222 and second tire assembly 224 move along their longitudinal axes in opposite directions until the EMD 208 reaches a second position 372 on first tire assembly 222 and a third position 375 of second tire assembly 224. The controller will automatically reset first tire assembly 222 and second tire assembly 224 along their respective longitudinal axes 242, 246 to a reset position. If the user continues to provide instructions to rotate EMD 208 in the same first direction as or after the first tire assembly and second tire assembly reaches or reached the second and third positions respectively, the controller will automatically set the reset position to a third location 374 on the first tire assembly and a second position 372 on the second tire assembly. In this manner tire assemblies 222 and 224 are in the position to continue rotating EMD 208 in the first direction for a greater number of rotations than if the reset position was the center positions 370 and 371. Stated another way the first tire assembly 222 and second tire assembly mover relative to one another along their respective longitudinal axes 242 and 246 between a first extended position illustrated in FIG. 10B and a second extended position opposite the first extended position illustrated in FIG. 10C. In the first extended position the upper portion of first tire assembly 222 is proximate the lower portion of second tire assembly 224. IN the second extended position the lower portion of first tire assembly 222 is proximate the upper portion of second tire assembly 224.

In one embodiment the reset position is a function of the input device instructions including a duration of inactivity of the input device. Controller detects the duration of time that no instruction has been given to rotate the EMD. Once that duration reaches a predetermined time interval, the system automatically resets the first tire assembly 222 and second tire assembly 224 to an inactivity reset position. In one embodiment the inactivity reset position is a central position where the center portion of first tire assembly 222 is proximate the center portion of second tire assembly 224 such that first position 370 of the first tire assembly 222 is adjacent first position 371 of the second tire assembly 224. However, other inactivity reset positions may be used.

Referring to FIGS. 22A and 22B the drive mechanism 210 is described in greater detail. Drive mechanism 210 includes a base 212, actuation assembly 214 and EMD engagement mechanism 216. Base 212 includes the components of drive mechanism 210 that are reusable. Actuation assembly 214 is operatively secured within a cavity defined by base 212. A coupler mechanism 218 operatively connects actuation assembly 214 with the EMD engagement mechanism 216. In one embodiment base 212 includes a top plate AA and a bottom pate BB.

Coupler mechanism 218 includes a first support 268 and a second support 280 that extend outwardly of base 212 via shaft 272 and shaft 282 respectively. EMD engagement mechanism 216 includes a first tire assembly 222 and a second tire assembly 224. Tire assemblies 222 and 224 are located within a housing 220 that is operatively connected to base 212. EMD engagement mechanism 216 includes a first tire assembly 222 and a second tire assembly 224. In one embodiment first tire assembly 222 and second tire assembly 224 are identical. First tire assembly 222 includes a hub 226 supporting a tire 228 that is positioned about an external surface of hub 226. Similarly, second tire assembly 224 includes a hub 227 supporting a tire 229 that is positioned about an external surface of hub 227. Each tire 228 and 229 include a roller having a longitudinal axis about which the tire rotates. Tire 228 has an outer surface that contacts the EMD. In one embodiment the outer surface of each tire has a constant radius from a first end of the tire to the opposing second end of the tire. In one embodiment the radius of the outer surface varies along the longitudinal axis of the tire. In one embodiment the radius of the outer surface intermediate the two ends of the tire is greater than the radius of the outer center at the each of the two ends of the tire. In one embodiment the outer surface defines a prolate shape. In one embodiment the outer surface of the tires define a frusto conical shape or profile in which tires have a larger diameter proximate one free end of the tire than the other end of the tire. When the first tire and second tire grip an EMD therebetween the surfaces pressing against the EMD are substantially parallel to one another, while the surfaces of the tires that are not pressing against the EMD are not parallel. Referring to FIG. 22P tires having a conical shape compensate for deflections and clearances found in shafts 272, 282 and bearings (not shown but would be positioned in the apertures in first housing coupler 266 and second housing coupler 268). In the unpinched state, the conical tires would have parallel axes meaning that the surfaces would not be parallel. In the pinched state, the tire surfaces in the area of contact would be parallel. The angle of the cone is equal to the amount that the shafts are out of parallel due to shaft deflections and bearing clearances. In one embodiment the conical tires have an angle of between 0.1 and 10 degrees. In one embodiment the conical tiers have an angle of between 0.5-3.0 degrees.

Movement of tires 228 and 229 toward and away from each other grip and ungrip an EMD placed therebetween. As described herein movement of tires 228 and 229 about their longitudinal axis translates the EMD gripped therebetween and relative movement of tires 228 and 229 along the longitudinal axis of tires 228 and 229 rotate the gripped EMD about its longitudinal axis.

In one embodiment hub 226 includes a first portion 230 having an outer cylindrical shape and a second portion 232 having a frustoconical shape extending from the first portion 230 and terminating at a top end 234. A pair of engagement arms 236 extend from a bottom of first portion 230 and terminate with a hook barb shaped member 238 that operatively engages a portion of second support 268.

Referring to FIGS. 22C and 22D actuation assembly 214 provides three operational movements including rotational drive, axial drive, gripping/ungripping. In one embodiment clamping/unclamping drive is part of the gripping/ungripping mode or a separate fourth mode. That is the rotational drive mode rotates the EMD about its longitudinal axis. The axial drive mode drives the EMD along its longitudinal axis. The grip/ungrip and clamp/unclamp mode acts to both grip/ungrip a portion of the EMD between the two tires as well as to clamp/unclamp a portion of the EMD a distance from the two tires. In one embodiment there is no clamp.

A first motor 240 is operatively coupled to first tire assembly 222 providing rotational movement to first tire assembly 222 and therefore also tire 228 about a longitudinal axis 242 of first tire assembly 222. Control of first motor 240 from the workstation provides control of the linear movement of the EMD. In one embodiment, first motor 240 has an output shaft 290 operatively coupled to a first pulley 292. First pulley rotates with output shaft 290 and rotates a second pulley 270 via a belt 294. In one embodiment pulley 292 and 270 are gears that are connected either directly via gear teeth or through a gear chain having at least one additional gear connecting gear 292 and 270. In one embodiment, output shaft 290 is directly connected to shaft 272 or to tire assembly 222 with a coupler.

Referring to FIG. 22F a second motor 244 is operatively coupled to the first and second support 268, 280 to provide linear movement of the tire assemblies relative to one another. First tire assembly 222 moves along longitudinal axis 242 in a first direction and an opposing second direction and second tire assembly 224. Second tire assembly includes a longitudinal axis 246 spaced from and parallel to first tire assembly longitudinal axis 242. moves in equal distance and opposite direction along a second longitudinal axis 246 spaced from and parallel to the first longitudinal axis 242. Control of second motor 244 from the workstation provides control of the rotational movement of the EMD.

Referring to FIG. 22F and FIG. 22G a third motor 248 is operatively coupled to a clamp assembly 250 that is operatively coupled to a grip/ungrip mechanism 304 effecting tire assembly 216. As described herein control of the third motor 248 from the workstation provides resetting for the tire assemblies for discrete incremental rotation of the EMD about its longitudinal axis as well as loading and unloading the EMD.

Referring to FIG. 22A the linear drive of the actuation assembly first motor 240 in response to controls from the workstation rotates a pulley or gear 292. A belt or gear train 294 operatively rotates a second pulley or gear operatively connected to first engagement member 218 secured to first tire assembly 216. Rotation of an output shaft of the first motor 240 in a clockwise direction results in first tire assembly rotating in a clockwise direction about the longitudinal axis 242 of the first tire assembly 222. Rotation of the output shaft of the first motor 240 in a counterclockwise direction results in the counterclockwise rotation of the first tire assembly 222. In one embodiment first tire assembly 222 and second tire assembly 224 are biased toward one another such that rotation of first tire assembly 222 in the clockwise and counterclockwise orientation results in the counterclockwise and clockwise rotation respectively of the second tire assembly 224. This motion can happen in one embodiment because the tires are in contact with each other and in one embodiment because the idler tire is being driven by the EMD. The insertion direction is defined as the direction that an EMD will move along its longitudinal axis from the proximal end of housing 220 toward the distal end of housing 220 when first tire assembly 222 is rotated counterclockwise. The insertion direction will move the EMD further into a patient's vasculature. In a withdraw direction an EMD will move along its longitudinal axis in a direction from the distal end of housing toward the proximal end of housing 220 when first tire assembly 222 is rotated clockwise. In one embodiment a longitudinal axis of the first motor output shaft is offset from the longitudinal axis 242 of the first tire assembly 222. In one embodiment the longitudinal axis of the first motor output shaft is offset from both the longitudinal axis 242 of the first tire assembly 222 and the longitudinal axis 246 of the second tire assembly.

Referring to FIGS. 22C and 22D rotational drive includes a coupler 252 operatively connecting second motor 244 with first coupler mechanism 218 and second coupler mechanism 254. In one embodiment second motor 244 has an output shaft that is connected to coupler 252. Coupler 252 in one embodiment is a link being connected to the output shaft of second motor 244 at a center connector 254. Rotation of the output shaft of second motor 244 results in rotation of coupler 252 about the axis of the output shaft of second motor 244. A first end 256 of coupler 252 is operatively secured to the first tire assembly 222 and the second end 258 of coupler 252 is operatively secured to the second tire assembly 224.

Referring to FIG. 22D, a first end 262 of a rod 260 is pivotally secured to first end 256 of coupler 252. A second end 264 of rod 260 is secured to a first housing coupler member 266. Referring to FIGS. 22M and 22N coupler mechanism 218 include a first support or first coupler 268 having a shaft portion 272 connected to first housing coupler member 266 such that movement of first housing coupler 266 along the longitudinal axis 242 results in longitudinal movement of the first support 268 in the same direction and in equal distance as the first housing coupler. A second rod 356 includes a first end 358 pivotally secured to a second end 258 of coupler 252. A second end 360 of second rod 356 is secured to second housing coupler 288. First end 358 and second end 360 are secured to coupler 252 and coupler 288 with a rod end providing necessary swivel for the additional degrees of freedom required when the tire assemblies are being moved between the gripped and ungripped positions. Rotation of the output shaft of second motor 244 in a first direction results in rotation of rocker 252 in a first direction which results in movement of rod 260, first housing coupler 266, coupler 268 and first tire assembly in a first direction along longitudinal axis 242 and movement of second rod 356, coupler 280 and second tire assembly 224 in a second direction along longitudinal axis 246 where the second direction is in direction parallel to and opposite the first direction. First housing coupler 266 and second housing coupler 288 move in a linear direction along longitudinal axis 242 and 246 respectively along shafts 354 and 356 respectively

First housing coupler 266 includes a center region housing a pulley or gear 270 secured to a shaft 272 of first support 268. First support 268 includes a portion extending from shaft 272 away from housing coupler 266 having a first region 274 and a second frustoconical portion 276 respectively receiving portions 230 and 232 of first tire assembly 222. First region 274 has a diameter that is greater than the diameter of shaft portion 272. Referring to FIG. 22N a shelf region 278 (also referred to as a shoulder region) extends radially outward from shaft portion 272 a distance equal to the difference between the radius of the first region 274 and the radius of the shaft portion 272. As described herein barbs 238 removably engage shelf region 278 to removably secure first tire assembly 222 from first support 268. Shaft 272 is free to rotate within first housing coupler in response to rotation of the output shaft of first motor 240. In one embodiment the diameters of shaft 272 and first region 274 are the same and shoulder area is defined by an inwardly extending groove in one of the shaft 272 and first region 274. In one embodiment outwardly extending ridge may extend from the shaft or first region 274 that the tire assembly may be releasably secured to.

A second support or coupler 280 includes a shaft portion 282, a conical support region 284, a frustoconical portion 286 and a shelf region 279. Shelf region 279 extends from shaft portion 282 a distance equal to difference between the radius of the first region 284 and the radius of the shaft portion 282. As described herein barbs 239 removably engage shelf region 278 to removably secure second tire assembly 224 from second support 280. Shaft 282 is free to rotate about longitudinal axis 246 within a second housing coupler 288 in response to rotation of the output shaft of first motor 240. As discussed in further detail herein, in one embodiment installation and/or removal of first tire assembly 222 and second tire assembly 224 is accomplished via automated process controlled by the controller.

In one embodiment first motor 240 is operatively secured to first housing coupler 266 such that first motor 240 moves along with first housing coupler 266. In one embodiment output shaft 290 of first motor 240 includes a key shape that engages pulley 292 such that pulley 292 moves with first housing coupler 266 while first motor 240 is fixed relative to base 212. In one embodiment first motor 240 and pulley 292 moves in direction parallel to the longitudinal axis of shaft 272 with first housing coupler 266.

Referring to FIG. 22F an output shaft of second motor 244 is pivotally coupled to coupler 252 at a position between the first end and the second such that clockwise rotational movement of the second motor output shaft results in a generally upward movement of the first tire assembly 222 and generally downward movement of the second tire assembly 224. Coupler 252 is also referred to herein as a rocker as its rocks or pivots about center 254.

Referring to FIG. 22G-22J a holding clamp 250 releasably clamps a portion of EMD 208 spaced from the first tire and the second tire along the longitudinal axis of EMD 208. Referring to FIG. 22G a clamp assembly 250 includes a cam 298 operatively rotated by third motor 248. Cam 298 has an outer circumference with an engagement portion 300 that engages a clamping pad 302 the cam 298 is rotated about a rotation axis through a certain degree of rotation (in one example through 90 degrees of rotation). A grip/ungrip mechanism 304 is operatively connected to the clamp assembly 250 to move second tire assembly 224 toward and away from first tire assembly 222 to grip and ungrip the EMD respectively therebetween. The grip/ungrip mechanism includes a link first crank 306 operatively connected to the cam 298 via a shaft 308 and coupler 310. In one embodiment cam 298 is permanently affixed to a portion of the coupler 310. First crank 306 is operatively connected to third motor output shaft 312. First crank 306 is pivotally connected to a tie rod 314 having a slot 316. A second rocker arm 318 having a follower 320 is positioned within slot 316. Second rocker arm 318 is connected to an eccentric housing 322 that has a hole 324 off centered. Eccentric housing 322 has an outer wall with an outer diameter defining an outer surface and inner diameter defining an inner surface, wherein the outer surface and inner surface do not define concentric cylinders. Shaft 282 of second support 280 extends through hole 324 such that clockwise and counterclockwise rotation of eccentric housing 322 by movement of rocker arm 318 results in second tire assembly 224 being moved toward and away from first tire assembly 222. An inner seal is positioned within opening 324 of eccentric housing 322 providing a seal between shaft 282 and the inner surface of eccentric housing 322 during rotation of shaft 282 within eccentric housing 322 and movement of eccentric housing upon movement of second rocker arm 318. A second outer seal (not shown) is positioned between eccentric housing 322 and plate AA or base AA. Second outer seal allows eccentric housing 322 to be sealed relative to plate AA as the eccentric housing 322 rotates within an aperture in plate AA.

Referring to FIG. 22O, in one embodiment eccentric seal assembly is between second shaft 282 and plate AA of the base housing operatively sealing the second shaft 282 from the base as the second shaft 282 is moved away from and toward the second shaft. In one embodiment eccentric housing assembly is positioned between the first shaft and the first shaft moves toward and away from the second shaft.

In one embodiment a drive module includes a first actuator operatively rotating a first shaft and/or a second shaft. A second actuator operatively translates the first shaft along its longitudinal axis relative to the second shaft from a first position to a second position. A first tire assembly is removably attached to the first shaft and a second tire assembly removably attached to a second shaft. An EMD having a longitudinal axis being positioned at a first location between the first tire assembly and the second tire assembly, wherein rotation of the first shaft translates an EMD along its longitudinal axis between the first tire assembly and the second tire assembly; and rotation of the second shaft rotates the EMD about its longitudinal axis. A third actuator operatively moves the first tire assembly toward and away from the second tire assembly gripping and ungripping the EMD from between the first tire assembly and the second tire assembly. A holding clamp releasably clamps a portion of the EMD spaced from the first tire and the second tire along the longitudinal axis of the EMD. In one embodiment the third actuator automatically moves the first shaft away from the second shaft and the second actuator automatically moves the first shaft back to a reset position when the first shaft reaches a predetermined distance from the first position, and the holding clamp automatically clamps the EMD while the first shaft is moved away from the second shaft. In one embodiment the third actuator operatively moves the clamp between a clamping position to an unclamped position.

In one embodiment drive mechanism operates in at least three different modes. In a drive mode the clamp is an unclamped position with respect to the EMD and the first tire assembly and second tire assembly grip the EMD therebetween. In a reset mode, the clamp is in a clamped position with respect to the EMD and first tire assembly being is in an ungripped position. In an exchange mode, the clamp is in the unclamped position and the tire engagement mechanism being in an the ungripped position.

Referring to FIG. 22G in a first position, clamp assembly 250 is in an unclamped position and grip/ungrip assembly 304 is in a gripped position. In this first position cam engagement portion 300 of cam 298 is spaced from the EMD and the clamping pad 302. In this first position, the EMD is free to rotate about its longitudinal axis and move along its longitudinal axis without being impeded by the cam 298 and cam support 300.

In the reset mode, prior to ungripping the EMD from between the first tire and the second tire the clamp is moved the clamped position, so the EMD is secured from movement at two locations. Stated another way a first portion of the EMD is secured from rotation and linear movement at the clamp and a second portion of the EMD is secured from rotation and linear movement between the gripped first tire and second tire. After the clamp is moved to the clamped position, the first tire and/or second tire is moved to the ungripped position. By following this sequence of first clamping and then ungripping any force or torque in the EMD does not recoil resulting in loss of positional control of the EMD such as movement of the EMD within the drive and/or proximal portion. It is desirable to maintain the existing torque in the EMD while resetting to continue rotation of the EMD. The EMD acts like a spring and failure to maintain the existing torque and/or force will result in the EMD springing back to a position once the torque and/or force is released. The reset mode allows the first tire and second tire to be repositioned to allow continued rotation of the EMD in the same direction. By way of example an EMD is initially placed located in the middle of the first tire and the middle of the second tire where the first tire and second tire are generally aligned in a neutral position. In the neutral position the center line of the first tire is in contact with the centerline of the second tire.

To rotate the EMD in a first direction about its longitudinal axis the first tire and second tire move in equal and opposite direction along their respective longitudinal axes. The first tire and second tire are able to continue moving in equal and opposite directions until the EMD is positioned at a terminal end of the first tire and a terminal end of the second tire. Any further movement of the tires relative to one another would result in the EMD being no longer between the first tire and second tire. To allow the tires to continue to rotate the EMD about its longitudinal axis in the first direction, the EMD is clamped and then released from between the tires and the tires move back to the neutral position. The amount of throw or distance that the wheels can move in equal and opposite directions is the distance between the neutral position and the terminal ends of the tires. When the throw falls below a predetermined amount the drive mechanism automatically resets to the neutral position or other predetermined position. In one embodiment a wire guide (Not shown) prohibits the EMD from moving from between the tires during rotation of the EMD. Wire guide also acts to trigger automatic reset of the tires if the EMD moves to the terminal edges of the tires. (Passive wire guide retains EMD between the Tire surface to maintain the EMD such a guidewire centered between the terminal ends of the tires during reset as well as to prohibit the EMD from falling off of the tires)

In one embodiment in the exchange mode there is no need to clamp the EMD prior to ungripping the tires to avoid recoil since the EMD will be removed from the drive mechanism.

Referring to FIG. 22H in a second position, clamp assembly 250 is in a clamped position and the grip/ungripped assembly 304 is in a gripped position. The cam engagement portion 300 is at the start of a dwell where it is clamping the EMD. The cam follower 320 of the second rocker arm 318 is now at the end of the dwell in the slot 316 of tie rod 314 such that the second tire assembly is engaged with the first tire assembly such that the EMD is gripped between the tire of the first tire assembly 222 and the tire of the second tire assembly 224.

Referring to FIG. 22I in a third position, the clamp assembly 250 remains in a clamped position and the grip/ungrip assembly 304 is in an ungripped position such that the EMD is not gripped between the tire of the first tire assembly 222 and second tire assembly 224. In this third position the cam engagement portion 300 is still in contact with the clamping pad 302 and is at the end of the dwell where it holds the EMD. The tire cam follower 320 rotates the eccentric which moves tire assembly 224 from tire assembly 222.

Referring to FIG. 22J in a fourth position, clamp mechanism 250 is in an unclamped position and the grip/ungrip mechanism 304 is in an ungripped position. In this fourth position the EMD is not clamped by either the holding clamp or gripped between the first tire assembly 222 and the second tire assembly 225. In this fourth position the engagement portion 300 is not applying a clamping force to the EMD and bushing 322 is rotated such that second tire assembly 225 is spaced from first tire assembly 222 such that there is a gap between the tires allowing the EMD to be removed from drive mechanism 210.

Referring to FIG. 22E, housing 220 is a disposable cassette that is operatively removably connected to a base 212. In one embodiment first support coupler 268, second support coupler 280 and cam coupler 310 are positioned above top surface 326 to respectively removably receive the first tire assembly 222, second tire assembly 224 and cam 298. A sterile barrier extends between housing 220 and the top surface 326 of base 212. In one embodiment, first coupler 268, second coupler 280 and cam coupler 310 are also included in the housing and inserted into the actuation assembly 214 via shafts 272, 282 and 308 respectively.

Referring to FIG. 22M first tire assembly 222 and second tire assembly 224 are removably connected to coupler 268 and coupler 280 respectively. Referring to FIG. 22R second tire assembly 224 is attached to coupler 280 by attachment of moving coupler 280 along linear axis 246 in a first direction 336. The first direction is the direction along linear axis 246 in a direction away from base bottom 328 toward base top surface 326. The second direction is the direction along linear axis 246 opposite to the first direction. As coupler 280 is moved in the first direction tire assembly 224 is restrained from moving along longitudinal axis 246 in the first direction by a restraint 332. In one embodiment restraint 332 is a portion of a cover 334 of housing 220. In one embodiment the restraint 332 is a separate member independent of the cover such as a shipping clip. Although not illustrated in FIG. 22M first tire assembly 222 and second tire assembly 224 are located within housing 220. As top 330 of coupler 268 moves in the first direction, barbs 239 are biased in a direction away from longitudinal axis 246 until barb 239 clears the shelf region 278 of coupler 268. Once barb 239 clears the shelf region 278, the barbs are biased toward longitudinal axis 246. A spring 340 biases a plunger 342 against a bottom surface 346 of the top of the second tire assembly 224. The spring 340 maintains the second tire assembly 224 in a fixed position relative to the coupler 280, such that rotation of coupler 280 and/or linear movement of coupler 280 results in equal rotation and/or linear movement respectively of second tire assembly 224. In one embodiment the spring force is set with a force that is greater than the force to actuate the tires longitudinally so that the tire moves relative to the shaft with no backlash.

Movement of coupler 280 in the first direction is accomplished by control of second motor 244 by a controller. Attachment of first tire assembly 222 to first coupler 268 is accomplished in the same manner as attachment of second tire assembly 224 to second coupler 280. In one embodiment a single second motor 244 controls the movement of first coupler 268 and second coupler 280 along first longitudinal axis 242 and second longitudinal axis 246 respectively, such that movement of second coupler in the first direction, results in the first coupler moving in an equal distance in a second direction. In this embodiment, the tire assemblies are attached to their respective couplers one at a time. Stated another way the tire assemblies are attached in series such that there is a time lapse between the attachment between the one tire assembly and the other tire assembly.

In one embodiment second motor 244 includes two separate motors independently controlling the first coupler and second coupler respectively. In the embodiment in which there are two separate motors it is possible to attach first tire assembly 222 and second tire assembly 224 to their respective couplers simultaneously.

Referring to FIG. 22S-22T removal of first tire assembly 222 and second tire assembly 224 from respective couplers 268 and 280 occurs by activating second motor 244 such that coupler 280 moves in the second direction towards top surface 326 of base 212. A beveled portion 348 of barbs 239 of second tire assembly 224 contacts a boss 350 that biases barbs 239 in a direction away from longitudinal axis 246 until barbs 239 fully clears shelf portion 288. Spring 340 biases the second tire assembly in a first direction that allow second tire assembly to be removed from second coupler 280. The first tire assembly 222 is similarly removed from first coupler 268. In one embodiment boss 350 is an integral portion of base 212 extending from top surface of base 212 and in one embodiment boss is a separate member operatively secured to base 212.

Referring to FIG. 22U in one embodiment couplers 268 and 280 do not include a spring and plunger, rather first tire assembly 222 includes a spring member 352 operatively connected to the first tire assembly 222 such that spring 352 acts to maintain connection of the first tire assembly to the first coupler such that the first tire assembly moves along and about longitudinal axis 242 equally with movement of the first coupler. In this embodiment spring 352 is part of the disposable portion that has a single use.

Drive mechanism 210 includes one or more pairs of tires that grip an EMD between them. First tire 228 and second tire 229 of the pair of times are rotated to drive the EMD linearly and tires 228 and 229 are moved axially in opposite directions to drive the EMD in rotation. Drive mechanism 210 include an actuation assembly 214 that includes a number of integrated mechanisms to rotate the tires, translate the tires axially and to ungrip the tires. A rotation mechanism provides rotation of the tires by operatively coupling a first motor directly to the tire assembly directly or indirectly via a belt/gears. In one embodiment the rotation mechanism is mounted onto housing coupler 266 along a linear guide system which moves the tires and rotational motors vertically. The linear guide could include the housing coupler having a bushing riding on rods 258. However, other linear guides known in the art may be used. To move the first housing coupler 266 and second housing coupler 288 on the linear rails or shafts 362 and 364 respectively, there are connecting rods 260 and 356 pivotally secured to a rocker 252 mounted to an output shaft of second motor 244. To grip and ungrip the tires between tires 228 and 229 a third motor 248 operatively rotates an eccentric member 322 having an offset aperture 324 receiving one of the shafts of the first coupler and second coupler such that rotation of the bushing results in moving tires 228 and 229 away from one another. The tire assemblies 222 and 224 are located within housing 220 such as cassette that loosely holds the tire assemblies in place for assembly onto the actuation hardware supported by base 212. The cassette 220 acts as a sterile barrier to cover the components within the base in combination with a drape. In one embodiment cassette the sterile barrier is used without a drape. The tire assemblies are fully supported by the couplers which requires a rigid connection to the tires both axially and rotationally. The rigid connection enables both rotation of the tires and vertical motion to enable rotation of the EMD. The connection between the tires and hardware is releasable to enable removal of the cassette.

In one embodiment, the shafts 272 and 282 and corresponding tire assemblies 222 and 224 are nominally tilted in the unloaded state by approximately 0.5-1 degree towards each other along their longitudinal axes so that the portion of the shafts proximate the shoulder region of the shafts are closer than the portion of the shafts distal the shoulder region. The amount by which the shafts are tilted corresponds to the amount of deflection of the components and the clearance in bearings and bushings so that when the tires are in the gripped state and correspondingly loaded and, the rotational axes of the tires are substantially parallel. This ensures that small diameter (as low as 0.010″) of the elongate medical devices are well-gripped by the tires and that there are no clearances due to a lack of parallelism when loaded in the gripped state. In one embodiment the longitudinal axis of the bearings in first housing coupler 362 are tilted relative to the longitudinal axis of the bearings in second housing coupler 364 or stated another way the longitudinal axis of shafts 272 are not parallel to the longitudinal axis of shafts 282. In one embodiment the angle between the longitudinal axis of the bearings supporting shaft 272 and shaft 282 is greater than 0 degrees and less than 90 degrees. The tilt of shafts 272 and 282 are set by the location of relative angle of the longitudinal axes of bearings 362 and 364.

In one embodiment robotic drive system includes a first actuator 240 operatively rotating a first shaft 272 and/or a second shaft 282 and a second actuator 244 operatively translating the first shaft 272 along its longitudinal axis relative to the second shaft 282 from a first position to a second position. A first bearing having a first longitudinal axis that supports the first shaft 272 and a second bearing having a second longitudinal axis supports the second shaft 282; and the first longitudinal axis and the second longitudinal axis being non-parallel. A first tire assembly 222 is removably attached to the first shaft 272 and a second tire assembly 224 is removably attached to a second shaft 282. A third actuator 248 operatively moves the second tire assembly 224 toward and away from the first tire assembly 222 gripping and ungripping an EMD having a longitudinal axis from between the first tire assembly and the second tire assembly. In one embodiment first bearing is positioned within first housing coupler 266 and second bearing is positioned within second housing coupler 268. However, first bearing and second bearing may be positioned elsewhere. For example, second bearing may be the eccentric bearing assembly 322. In one embodiment the first longitudinal axis of the first bearing and the second longitudinal axis of the second bearing intersect forming an acute angle at an intersection point, wherein the first tire assembly and the second tire assembly are intermediate the intersection point and the first bearing and the second bearing.

In one embodiment molded in clips at the bottom of the tire assemblies clip under a lip on the coupler such as the shelf region 278. To deal with the tolerance stack up which will necessarily involve some amount of backlash, a spring-loaded plunger is be used at the top of the coupler will ensure the clips are always in tension. For releasing the tire assemblies, the rotation mechanism can be actuated, and the clips hit a lip designed to release them and force the tire off. Once one tire assembly is off, it will float up when the other tire is released. For the initial installation, restraint 332 is a shipping clip located within housing 220 is used to hold the tires down so that both tire assemblies can be snapped in but have them still be removable by the system.

In one embodiment, a robotic system includes a base 212 having a first actuator 240 and a cassette 220 housing that is removably connected to the base 212. A pair of tires 222, 224 are within the cassette 220. A robotic actuator moves first shaft 272 and 282 to operatively engage first tire 222 and second tire 224 on the first shaft 272 and second shaft 282 extending from the base 212 into cassette 220. In one embodiment the robotic actuator operatively disengages the pair of tires from the first shaft and/or second shaft. In one embodiment more than one pair of tires are positioned within cassette 220 and are operatively engaged and disengaged from respective shafts.

Rotation of the EMD occurs by moving tires 228 and 229 in opposite directions. Since the upward and downward movement of tires 228 and 229 is a fixed distance, in order to continue rotating the EMD in a same direction the tires need to be reset. Resetting the rotation capabilities of the tires includes incorporating a separate brake clamp that holds the EMD when tires 228 and 229 can be ungripped and then returned to the desired position after reset. The brake clamp includes a cam 298 with an engagement portion 300 and a clamp support 302.

Cam 298 is rotated by a motor that is controlled by the controller. In one embodiment the motor used to rotate cam 298 is the third motor 248 that is also used to grip and ungrip the tires from one another. In one embodiment motor 248 is operatively connected to both the brake mechanism and the grip/ungrip mechanism to coordinate the timing of the brake of the EMD and the grip/ungrip of the EMD from between the tires 228 and 229. As discussed herein first tire assembly via a first coupler 268 is mounted on an eccentric bushing 322 so that the first tire assembly can be swung away from the second tire assembly using rotation. The cam has a rocker arm that is linked to another rocker arm on the eccentric tire release by a tie rod. By linking these, as the cam is engaged with the clamp, the tires can be ungripped.

The drive 210 can be defined to have 3 distinct capabilities: driving, resetting, and exchanges. In the drive position, the cam is disengaged from the EMD and cam support and the follower 320 is riding free in the slot 316 so that the tires are gripped together by a spring force. In one embodiment a torsion spring (not shown) is operatively secured to the eccentric 322 and the base. In one embodiment a lever (not shown) is operatively coupled to the base with a linear spring in either compression or tension. Only rotational motion is used to grip and ungrip, accordingly, in one embodiment sealing between the base and the shafts is accomplished with a rotary shaft seal on the eccentric.

In the resetting position cam 298 fully clamps the EMD between the cam engagement portion 300 and the clamping pad 302 thus setting the brake before the follower 320 contacts the end of the slot 316. A dwell on the cam allows the cam to stay fully engaged clamping the EMD as the tires 228 and 229 are ungripped enough for reset. Tires are reset by activating second motor 244 moving the first tire assembly and second tire assembly to a position to continue rotation of the EMD in the desired direction.

In the exchange position cam 298 is positioned such that the cam is not clamping the EMD between the engagement portion and the cam support and the first and second tires are spaced from one another in the ungripped position. In this orientation the EMD is free to be removed from the drive mechanism 210.

In one embodiment a manual release is provided to release both the cam from locking the EMD and to ungrip tires 228 and 229. The manual release overrides the controller controlling the motors in the case of a power outage or other need to quickly release the EMD from the clamp and tires. In one embodiment, a portion of the cam is operatively connected to a handle accessible to a user to manipulate such as by twisting. This design feature could be a key sufficiently large to enable a user to grip the key with the user's hand, which is easy to grip. In one embodiment only the first tire assembly moves in an up and down direction, while the second tire assembly is in a fixed up down position. In this embodiment, the mechanism described above is retained, but one of the 2 tie rods that operatively secured to rocker 252 is removed. In this mode to obtain the same amount of EMD rotation, motor 244 turn twice as much as the embodiment in which both tie rods are connected.

Although the present disclosure has been described with reference to example embodiments, workers skilled in the art will recognize that changes may be made in form and detail without departing from the spirit and scope of the defined subject matter. For example, although different example embodiments may have been described as including one or more features providing one or more benefits, it is contemplated that the described features may be interchanged with one another or alternatively be combined with one another in the described example embodiments or in other alternative embodiments. The present disclosure described is manifestly intended to be as broad as possible. For example, unless specifically otherwise noted, the definitions reciting a single particular element also encompass a plurality of such particular elements.

What is claimed is:

1. An EMD drive system comprising:
an on-device adapter removably fixed to a shaft of an EMD;
the on-device adapter received in a cassette;
the cassette removably secured to a drive module; and
the drive module operatively coupled to the on-device adapter to move the on-device adapter and EMD together.

2. The EMD drive system of claim 1, wherein the on-device adapter is moved in translation.

3. The EMD drive system of claim 2, wherein the on-device adapter is moved in rotation about a longitudinal axis of the on-device adapter.

4. The EMD drive system of claim 3, wherein the on-device adapter includes a collet.

5. The EMD drive system of claim 4, wherein the collet includes a first member moving along and/or or about a longitudinal axis of a second member to pinch the EMD.

6. The EMD drive system of claim 4, wherein the on-device adapter includes an engagement portion engaged with and driven by a drive member in the cassette to rotate the on-device adapter.

7. The EMD drive system of claim 6, wherein the on-device adapter includes a surface that is supported by a bearing member in the cassette.

8. The EMD drive system of claim 7, wherein the on-device adapter includes a thrust bearing surface preventing translational movement relative to a portion of the cassette.

9. The EMD drive system of claim 6, wherein the on-device adapter includes a luer connector.

10. The EMD drive system of claim 2, wherein the on-device adapter includes a quick clamp releasably engaging a collet.

11. The EMD drive system of claim 10, wherein the quick clamp quickly connects and/or releases the collet.

12. The EMD drive system of claim 10, wherein the quick clamp releasably engages the collet without tools.

13. The EMD drive system of claim 10, wherein the quick clamp includes a lever movable from a first position to clamp the collet thereto to a second position to unclamp the collet thereto.

14. The EMD drive system of claim 4, wherein the EMD is removably received in the collet in a radial direction and the collet is removably received and positioned in the cassette.

15. The EMD drive system of claim 4, wherein the EMD is removably received in the collet in an axial direction and the collet is removably received in the cassette.

16. The EMD drive system of claim 4, wherein the EMD is removably received in the collet in a radial direction and the collet is non-removably positioned within cassette.

17. The EMD drive system of claim 4, wherein the EMD is removably received in the collet in an axial direction and the collet is non-removably positioned within the cassette.

18. The EMD drive system of claim 1, wherein the drive module includes an actuator operatively coupled to a drive coupler;
a drive member in the cassette being operatively coupled to the drive coupler; and
the drive module being operatively coupled to a rail or linear member and including a second actuator that translates the drive module along the rail.

19. The EMD drive system of claim 1, wherein the EMD is a guidewire.

20. The EMD drive system of claim 1, wherein the EMD is a catheter having a hub at a proximal end of the catheter and a shaft extending from the hub toward a distal portion of the catheter, wherein the shaft is more flexible than the hub.

21. A robotic system comprising:
a collet having a first portion having a first collet coupler connected thereto and a second portion having a second collet coupler connected thereto;
an EMD being removably located within a pathway defined by the collet; and
a robotic drive including a base having a first motor and a second motor operatively continuously coupled to both the first collet coupler and the second collet coupler respectively to operatively pinch and unpinch the EMD in the pathway and to rotate the EMD.

22. The robotic system of claim 21, wherein the first motor and the second motor rotate the first collet coupler and the second collet coupler at different speeds and/or different directions.

23. The robotic system of claim 21, further including a cassette removably secured to the base, the collet being positioned within the cassette, the first collet coupler and the second collet coupler being respectively coupled to the first motor and the second motor via a first drive coupler and a second drive coupler positioned within the base.

24. The robotic system of claim 21, wherein the EMD does not rotate while the EMD is being pinched and unpinched.

25. The robotic system of claim 21, further including a third motor operatively coupled to the collet to translate the collet and EMD along a longitudinal axis of the collet.

26. The robotic system of claim 25, wherein the first motor and second motor are fixed relative to the base during translation of the collet and EMD.

27. The robotic system of claim 26 wherein the collet includes a first gear and a second gear that remain engaged with the first motor and second motor during translation of the collet.

28. The robotic system of claim 25, wherein the first motor and second motor are fixed relative to the collet during translation of the collet and EMD.

29. The robotic system of claim 21, wherein the robotic system has a pinch/unpinch mode, a rotation mode, and a translation mode.

30. The robotic system of claim 29, wherein at least two of the pinch/unpinch mode, rotation mode and translation mode occur simultaneously.

31. The robotic system of claim 30, further including a clamp to selectively clamp and unclamp the EMD, wherein the clamp is in an unclamped position and the collet is in an unpinched state during an exchange mode.

32. The robotic system of claim 31 wherein the clamp includes a pair of tires.

33. A collet comprising:
an inner member defining a pathway receiving an EMD;
an outer member; and
a plurality of engagement members releasably engaging the EMD as the inner member is moved relative to the outer member.

34. The collet of claim 33, wherein the engagement members sequentially engage the EMD.

35. The collet of claim 33, wherein the engagement members are offset circumferentially about the EMD.

36. The collet of claim 33, wherein the engagement members are offset axially.

37. The collet of claim 32, wherein a first engagement member is positioned 180 degrees from a second engagement member.

38. The collet of claim 33, wherein the engagement members are independent and not directly connected to one another.

39. The collet of claim 33, wherein the engagement members are biased with a spring member toward the pathway.

40. The collet of claim 33, wherein the engagement members are biased with a spring member away from the pathway.

41. The collet of claim 33, wherein movement of the inner member relative to the outer member is rotational.

42. The collet of claim 33, wherein movement of the inner member relative to the outer member is translational.

43. The collet of claim 33, wherein movement of the inner member and outer member relative to one another is robotic.

44. The collet of claim 33, wherein movement of the inner member and outer member relative to one another is manual.

45. The collet of claim 33 wherein the engagement members are offset radially about the EMD.

46. An EMD drive system comprising:
a collet including a collet first member having a first engagement portion;
the collet including a collet second member that is driven; and
a collet engagement member having a second engagement portion;
the collet first member and the collet engagement member moving between an engaged position and a disengaged position;
the first engagement portion engages the second engagement portion as the collet first member and collet engagement member are moved to the engaged position along a longitudinal axis of the collet;
wherein rotation of the collet first member with respect to the collet second member in a first direction in the engaged position pinches an EMD within the collet and rotation of the collet first member with respect to the collect second member in a second direction opposite the first direction unpinches the EMD within the collet.

47. The EMD drive system of claim 46, wherein the first engagement portion includes a plurality of splines that extend circumferentially about at least a portion of the collet first member, and the second engagement portion includes a plurality of members operatively engaging the plurality of splines.

48. An EMD robotic drive system rotating and translating an EMD with reset instructions, comprising:
a drive module controlled by a control system, the drive module including:
a first actuator operatively rotating a first shaft and/or a second shaft;
a second actuator operatively translating the first shaft along its longitudinal axis relative to the second shaft from a first position to a second position;
a first tire assembly operatively attached to the first shaft;
a second tire assembly operatively attached to a second shaft;
a third actuator operatively moving the first tire assembly toward and away from the second tire assembly gripping and ungripping an EMD having a longitudinal axis from between the first tire assembly and the second tire assembly;
wherein translation of the first shaft relative to the second shaft rotates the EMD about the longitudinal axis of the EMD, and rotation of the first shaft and/or the second shaft translates the EMD along the longitudinal axis of the EMD; and
a control system providing reset instructions to:
the third actuator to ungrip the EMD;
the second actuator to move the first tire assembly relative to the second tire assembly to a reset position; and
the third actuator to grip the EMD.

49. The EMD robotic drive system of claim 48, wherein the control system provides the reset instructions when the second position reaches a predetermined distance from the first position.

50. The EMD robotic drive system of claim 48, including an input device operatively providing input device instructions to rotate the EMD, the control system providing the reset instructions as a function of the input device instructions.

51. The EMD robotic drive system of claim 50, wherein the input device instructions include direction of rotation of the EMD.

52. The EMD robotic drive system of claim 50, wherein the input device instructions include a duration of inactivity of the input device.

53. The EMD robotic drive system of claim 48, further including an eccentric seal assembly between one of the first shaft and the second shaft and a base operatively sealing the first shaft or second shaft from the base as the first shaft or the second shaft is moved away from and toward the other of the first shaft and the second shaft.

54. The EMD robotic drive system of claim 48, further including a holding clamp releasably clamping a portion of the EMD spaced from the first tire assembly and the second tire assembly along the longitudinal axis of the EMD.

55. An EMD robotic drive system comprising:
a drive module including:
a first actuator operatively rotating a first shaft and/or a second shaft;
a second actuator operatively translating the first shaft along its longitudinal axis relative to the second shaft from a first position to a second position;
a first tire assembly removably attached to the first shaft;
a second tire assembly removably attached to a second shaft;
an EMD having a longitudinal axis being positioned at a first location between the first tire assembly and the second tire assembly, wherein rotation of the first shaft translates an EMD along its longitudinal axis between the first tire assembly and the second tire assembly; and rotation of the second shaft rotates the EMD about its longitudinal axis;
a third actuator operatively moving the first tire assembly toward and away from the second tire assembly gripping and ungripping the EMD from between the first tire assembly and the second tire assembly; and
a holding clamp releasably clamping a portion of the EMD spaced from the first tire assembly and the second tire assembly along the longitudinal axis of the EMD.

56. The EMD robotic drive system of claim 55, the third actuator automatically moves the first shaft away from the second shaft and the second actuator automatically moves the first shaft back to a reset position when the first shaft reaches a predetermined distance from the first position, and the holding clamp automatically clamps the EMD while the first shaft is moved away from the second shaft.

57. The EMD robotic drive system of claim 56, wherein the third actuator operatively moves the holding clamp between a clamping position to an unclamped position.

58. An EMD robotic drive system comprising:
a first actuator operatively rotating a first shaft and/or a second shaft;
a second actuator operatively translating the first shaft along its longitudinal axis relative to the second shaft from a first position to a second position;
a first tire assembly operatively attached to the first shaft;
a second tire assembly operatively attached to a second shaft;
a third actuator operatively moving the first tire assembly toward and away from the second tire assembly gripping and ungripping an EMD having a longitudinal axis from between the first tire assembly and the second tire assembly;
wherein translation of the first shaft relative to the second shaft rotates the EMD about the longitudinal axis of the EMD, and rotation of the first shaft and/or second shaft translates the EMD along the longitudinal axis of the EMD; and
wherein the first actuator moves with the first shaft as the first shaft is moved along its longitudinal axis away from a home position.

59. An EMD robotic drive system comprising:
a first actuator operatively rotating a first shaft and/or a second shaft;
a second actuator operatively translating the first shaft along its longitudinal axis relative to the second shaft from a first position to a second position;
a first bearing having a first longitudinal axis supporting the first shaft;
a second bearing having a second longitudinal axis supporting the second shaft; and the first longitudinal axis and the second longitudinal axis being non-parallel;
a first tire assembly removably attached to the first shaft;
a second tire assembly removably attached to a second shaft; and
a third actuator operatively moving the second tire assembly toward and away from the first tire assembly gripping and ungripping an EMD having a longitudinal axis from between the first tire assembly and the second tire assembly.

60. The EMD robotic drive system of claim 59, wherein the first longitudinal axis of the first bearing and the second longitudinal axis of the second bearing intersect at an intersection point forming an acute angle at a point, wherein the first tire assembly and the second tire assembly are intermediate the intersection point and the first bearing and second bearing.

61. The EMD robotic drive system of claim 58, the first tire assembly and the second tire assembly include an outer surface having a conical profile.

62. An EMD robotic drive system:
a base having a first actuator;
a cassette housing removably connected to the base,
a pair of tires within the cassette; and
the first actuator moving a first shaft and/or second shaft to operatively engage the pair of tires on the first shaft and the on the second shaft respectively extending from the base into the cassette.

63. The EMD robotic drive system of claim 62; wherein the first actuator operatively disengages the pair of tires from the first shaft and/or the second shaft.

64. The EMD robotic drive system of claim 62, further including at least a second pair of tires.

65. The EMD drive system of claim 46 wherein the collet first member and the collet second member are formed as a single component in which the collet first member and collet second member are compliantly connected.

66. A method of robotically moving an EMD comprising:
pinching a shaft of an EMD in an on-device adapter;
removably securing the on-device adapter into a cassette
removably securing the cassette to a drive module; and
robotically moving the on-device adapter and the EMD together in translation along a longitudinal axis of the EMD and/or rotation about the longitudinal axis of the EMD.

67. The method of claim 66 further including unpinching the EMD in the on-device adapter with an actuator when the on-device adapter is secured in the cassette.

68. The method of claim 67, wherein unpinching the EMD is robotically controlled with an actuator.
